(19) United States
(12) Patent Application Publication
(10) Pub. No.: US 2026/0053745 A1
(43) Pub. Date: Feb. 26, 2026

(54) METHODS OF PROPHYLAXIS OF CORONAVIRUS INFECTION AND TREATMENT OF CORONAVIRUSES

(71) Applicant: Curemark, LLC, Rye Brook, NY (US)

(72) Inventors: Joan M. Fallon, White Plains, NY (US); Matthew Heil, Sherman, CT (US); James J. FALLON, Armonk, NY (US); Janice HUGHES, Boulder, CO (US)

(21) Appl. No.: 19/372,744

(22) Filed: Oct. 29, 2025

Related U.S. Application Data
(63) Continuation of application No. 18/063,229, filed on Dec. 8, 2022, now Pat. No. 12,485,094.
Application No. 18/063,229 is a continuation of application No. 17/410,743, filed on Aug. 24, 2021, now Pat. No. 11,541,009.
(60) Provisional application No. 63/076,500, filed on Sep. 10, 2020.

(51) Int. Cl. A61K 9/1271; A61K 9/00; A61K 9/02; A61K 38/46; A61K 38/47; A61K 38/48

(52) U.S. Cl. CPC A61K 9/1271; A61K 9/0029; A61K 9/025; A61K 38/465; A61K 38/47; A61K 38/48

(57) ABSTRACT

Methods for prophylaxis, treatment, and reduction of infection, re-infection, and transmission rates of Coronaviruses and more particularly Coronavirus Disease 2019 (COVID-19) resulting from a SARS-CoV-2 viral infection with the use of a pharmaceutical preparation comprising one or more coated or uncoated digestive enzymes, such as pancreatic enzymes and porcine pancreatic enzymes are described herein.

### CROSS-REFERENCE

This application is a continuation of U.S. patent application Ser. No. 18/063,229, filed Dec. 8, 2022, which is a continuation of U.S. patent application Ser. No. 17/410,743, filed Aug. 24, 2021, now U.S. Pat. No. 11,541,009, which claims the benefit of U.S. Provisional Application No. 63/076,500, filed Sep. 10, 2020, which application is incorporated herein by reference in its entirety.

### SUMMARY OF THE INVENTION

### Urgent Unmet Need

All over the world, the COVID-19 pandemic is causing significant loss of life, disrupting livelihoods, and threatening the recent advances in health and progress towards global development goals highlighted in the 2020 World Health Statistics published by the World Health Organization (WHO).

From the start of reporting on Dec. 30, 2019, through 4:00 pm CEST Jun. 23, 2021, there have been 178,837,204 confirmed cases of COVID-19 resulting in 3,880,450 deaths.

Initially, the new virus was called 2019-nCoV. Subsequently, experts of the International Committee on Taxonomy of Viruses (ICTV) termed it the Severe Acute Respiratory Syndrome (SARS-CoV-2) virus as it is very similar to the one that caused the SARS outbreak (SARS-CoVs). The virus has been named SARS-CoV-2 and the disease it causes has been named Coronavirus Disease 2019 (COVID-19). On Jan. 31, 2020, the Department of Health and Human Services (HHS) issued a declaration of a public health emergency related to COVID-19, effective Jan. 27, 2020, and mobilized the Operating Divisions of HHS. In addition, on Mar. 13, 2020, the President declared a national emergency in response to COVID-19.

Communicable diseases are currently the leading cause of preventable deaths worldwide, disproportionately affecting resource-poor settings. Pandemic influenzas add to already unacceptable levels of morbidity and mortality from diarrhea, malaria, pneumonia, malnutrition, HIV/AIDS and tuberculosis, in addition to causing high maternal and neonatal death rates. A few key conditions cause 90% of deaths from communicable diseases: pneumonia (3.9 million deaths per year); diarrheal diseases (1.8 million); and malaria (1.2 million). Malnutrition is a significant contributing factor to this mortality. During a pandemic, these illnesses are likely to increase in resource-poor settings where chronically strained health systems would face even higher patient volumes, severe resource constraints, and absenteeism of critical staff.

Influenza pandemics have occurred in both the 21st and 20th Century. According to the world Health Organization the first influenza pandemic of the 21st century occurred in 2009-2010 and was caused by an influenza A(H1N1) virus. It was the first pandemic for which many WHO Member States had developed comprehensive pandemic plans describing the public health measures to be taken, aimed at reducing illness and fatalities. For the first time, pandemic vaccine was developed, produced and deployed in multiple countries during the first year of the pandemic.

While most cases of pandemic H1N1 were mild, globally it is estimated that the 2009 pandemic caused between 100,000-400,000 deaths in the first year alone. Children and young adults were disproportionately affected in comparison to seasonal influenza, which causes severe disease mainly in the elderly, persons with chronic conditions and pregnant women.

Three influenza pandemics occurred at intervals of several decades during the 20th century, the most severe of which was the so-called “Spanish Flu” (caused by an A(H1N1) virus), estimated to have caused 20 to 50 million deaths in 1918-1919. Milder pandemics occurred subsequently in 1957-1958 (the “Asian Flu” caused by an A(H2N2) virus) and in 1968 (the “Hong Kong Flu” caused by an A(H3N2) virus), which were estimated to have caused 1 to 4 million deaths each.

There is an urgent unmet need for Prophylaxis of SARS-CoV-2 Infection and Treatment of COVID-19. The current COVID-19 prevention and treatment guidelines are unsustainable in terms of human loss, economic impact, and the burden on the healthcare system. The economic impact on the world's economy is unprecedented. The coronavirus pandemic is already delivering significant economic data, with the U.S. unemployment rate reaching the worst number since the Great Depression. Further, with the resumption of normal business activities, if ever, coupled possibility of future recurrences of COVID-19 pandemic including possible future mutations of SARS-CoV-2, there exists significant uncertainty in the timing and magnitude of any economic recovery.

Long-term symptoms are known to exist with SARS-CoV-2 infection well after the virus has left the respiratory tract. According to the Mayo Clinic, COVID-19 symptoms may persist for months and there are multiple long-term health risks. The virus can damage the lungs, heart and brain, which increases the risk of long-term health problems. Individuals with long-term health issues are sometimes describe as “long haulers” and the conditions have been called post-COVID-19 syndrome or “long COVID-19.” These health issues are sometimes called post-COVID-19 conditions. They're generally considered to be effects of COVID-19 that persist for more than four weeks after you've been diagnosed with the COVID-19 virus. Older people and people with many serious medical conditions are the most likely to experience lingering COVID-19 symptoms, but even young, otherwise healthy people can feel unwell for weeks to months after infection. Common signs and symptoms that linger over time include: fatigue, shortness of breath or difficulty breathing, cough, joint pain, chest pain, memory, concentration or sleep problems, muscle pain or headache, fast or pounding heartbeat, loss of smell or taste, depression or anxiety, fever, dizziness when you stand, worsened symptoms after physical or mental activities.

Although COVID-19 is seen as a disease that primarily affects the lungs, it can damage many other organs as well. This organ damage may increase the risk of long-term health problems. Organs that may be affected by COVID-19 include the heart, lungs, brain, kidneys, and multi-symptom inflammatory syndrome. Lasting damage to the heart muscle from COVID-19 has been shown by imaging tests taken months after recovery from COVID-19, even in people who experienced only mild COVID-19 symptoms. This may increase the risk of heart failure or other heart complications in the future. Long-term damage to the lungs may be induced by the type of pneumonia often associated with COVID-19. Long-standing damage to the tiny air sacs (alveoli) in the lungs, resulting scar tissue, can lead to long-term breathing problems.

COVD-19 is known to cause long-term brain damage. Even in young people, COVID-19 can cause strokes, seizures and Guillain-Barre syndrome, a condition that causes temporary paralysis. COVID-19 may also increase the risk of developing Parkinson's disease and Alzheimer's disease. In addition, some adults and children experience multisystem inflammatory syndrome after they have had COVID-19. In this condition, some organs and tissues become severely inflamed.

COVID-19 is also known to cause blood clots and blood vessel problems. COVID-19 can make blood cells more likely to clump up and form clots. While large clots can cause heart attacks and strokes, much of the heart damage caused by COVID-19 is believed to stem from very small clots that block tiny blood vessels (capillaries) in the heart muscle. Other parts of the body affected by blood clots include the lungs, legs, liver and kidneys. COVID-19 can also weaken blood vessels and cause them to leak, which contributes to potentially long-lasting problems with the liver and kidneys. People who have severe symptoms of COVID-19 often have to be treated in a hospital's intensive care unit with mechanical assistance, such as ventilators, to breathe. Simply surviving this experience can make a person more likely to later develop posttraumatic stress syndrome, depression and anxiety.

Since much of the long-term effects of SARS-CoV-2 infection are still unknown, we can look to long-term effects seen in related viruses, such as the virus that causes severe acute respiratory syndrome (SARS) develop chronic fatigue syndrome, a complex disorder characterized by extreme fatigue that worsens with physical or mental activity, but doesn't improve with rest. The same may be true for people who have had COVID-19. There is a clear and urgent unmet need for treatments to accelerate the recovery from COVID-19 and mitigate and damage due to long-term effects. In addition, long haulers need a treatment to accelerate their recovery from COVID-19.

The present COVID-19 pandemic has necessitated the rapid innovation and deployment of strategies for the prophylaxis of infection and emergent treatments for patients. At present, there is no pre-exposure or post exposure prophylaxis agents for SARS-CoV-2 other than vaccines. In addition, there are limited recommended options for the treatment of COVID-19; currently approved medications include the antiviral Remdesivir and Dexamethasone. Treatments are symptomatic, and in addition to Remdesivir and Dexamethasone, oxygen therapy represents the major treatment intervention for patients with severe infection. Mechanical ventilation may be necessary in cases of respiratory failure refractory to oxygen therapy, whereas hemodynamic support is essential for managing septic shock.

The genetic sequence of SARS-CoV-2, the coronavirus that causes COVID-19, was published on 11 Jan. 2020, triggering an intense global R&D activity to develop a vaccine against the disease. The scale of the humanitarian and economic impact of the COVID-19 pandemic is driving evaluation of next-generation vaccine technology platforms through novel paradigms to accelerate development, and the first COVID-19 vaccine candidate entered human clinical testing with unprecedented rapidity on 16 Mar. 2020.

A striking feature of the vaccine development land-scape for COVID-19 is the range of technology platforms being evaluated, including nucleic acid (DNA and RNA), virus-like particle, peptide, viral vector (replicating and non-replicating), recombinant protein, live attenuated virus and inactivated virus approaches. Public information on the specific SARS-CoV-2 antigen(s) used in vaccine development is limited. Most candidates for which information is available aim to induce neutralizing antibodies against the viral spike (S) protein, preventing uptake via the human ACE2 receptor. However, it is unclear how different forms and/or variants of the S protein used in different candidates relate to each other, or to the genomic epidemiology of the disease.

The global vaccine R&D effort in response to the COVID-19 pandemic is unprecedented in terms of scale and speed. Given the imperative for speed, in early 2021 emergency use has been granted by the FDA for several vaccines. This represents a fundamental change from the traditional vaccine development pathway, which takes on average over 10 years, even compared with the accelerated 5-year timescale for development of the first Ebola vaccine. There are currently no COVID-19 vaccines authorized for children. In addition, the long-term effects of the emergency use authorized COVID-19 vaccines are unknown and, ultimately, they may not be suitable for children, those who wish to have children, pregnant women, or even adults or adolescents.

Even It is not clear that a vaccines alone will avoid another pandemic. Much will depend on the actual COVID-19 viral reproduction rates. Current estimates put SARS-CoV-2 Viral Basic Reproduction Number (R₀) near 2.7 where R₀ is utilized in predicting the extent of immunization that a population requires (P) if herd immunity is to be achieved, the spread of the infection limited, and the population protected against future infection. FIG. 1 illustrates the Basic Reproduction Number R₀ for a variety of infectious diseases including: Influenza H1N1 in 2009, Influenza in Spring 1918, Influenza H2N2 in 1957, Ebola Virus, Zika, SARS-CoV-2, HIV, SARS-CoV-1, Influenza in Autumn of 1918, MERS-CoV, Smallpox, Rhinovirus, Poliomyelitis, and Measles. Note that SARS-CoV-2 R₀ is only slightly less infectious that HIV, for which, despite an enormous effort, does not currently have a vaccine.

To prevent sustained spread of the infection the proportion of the population that has to be immunized (P) has to be greater than 1-1/R₀. The relation between P and R₀ is shown in FIG. 2. The fundamental relationship is given by the equation P=1-1/R₀. Thus, with a COVID-19 R₀ of approximately 2.7, a population will require an immunization P of approximately 62.96%. Given that the best-case immunization numbers for a mature H1N1 vaccine are 41% and that any COVID-19 vaccine will likely have less than 100% effectiveness, it is unlikely that a vaccine alone will suffice to prevent a future COVID-19 pandemic.

By way of recent example, the 2017 to 2018 influenza season in the United States was a high severity season. Circulation of influenza viruses was widespread for an extended period throughout the country. Influenza A(H3N2) viruses predominated but influenza A(H1N1)pdm09 and B viruses also circulated. The Centers for Disease Control and Prevention (CDC) has estimated that there were 48.8 million influenza illnesses, 959,000 hospitalizations, and 79,400 influenza-associated deaths during 2017-2018, the highest morbidity and mortality since the 2009 pandemic. Influenza vaccination is the primary strategy to prevent influenza illness and its complications. Recent reports estimate that 42% of the US population was vaccinated against influenza during the 2017 to 2018 season, the mid-season estimates of the effectiveness of influenza vaccine were 36% against all influenza A and B virus infections and 25% against A(H3N2) virus infection.

Overall, the vaccine effectiveness against outpatient, medically attended, laboratory-confirmed influenza was 38% (95% confidence interval [CI], 31% to 43%), including 22% (95% CI, 12% to 31%) against influenza A(H3N2), 62% (95% CI, 50% to 71%) against influenza A(H1N1)pdm09, and 50% (95% CI, 41% to 57%) against influenza B. It is estimated that influenza vaccination prevented 7.1 million (95% Credible Interval [CrI], 5.4 million to 9.3 million) illnesses, 3.7 million (95% CrI, 2.8 million to 4.9 million) medical visits, 109 000 (95% CrI, 39 000 to 231,000) hospitalizations, and 8000 (95% CrI, 1,100 to 21,000) deaths. Vaccination prevented 10% of expected hospitalizations overall and 41% among young children (6 months to 4 years). As of Jun. 10, 2021, approximately 43% of the US Population has been fully vaccinated. A remarkable achievement. However worldwide only 5.9% of the population is fully vaccinated.

In the US, it is likely that several factors have substantially contributed to help mitigate infections and deaths from COVID-19. COVID-19 occurred late in the influenza (flu) season. While seasonal influenza viruses are detected year-round in the United States, influenza viruses are most common during the fall and winter. The exact timing and duration of flu seasons can vary, but influenza activity often begins to increase in October. Most of the time flu activity peaks between December and February, although activity can last as late as May. In addition, the unprecedented shelter in place federal, state, and local mandates, the closure of all non-essential businesses, schools, and public gatherings, coupled the use of preventive measures such as social distancing and appropriate protective equipment and procedures significantly reduced the rate of spread of the disease.

Further compounding a reduced effectiveness of COVID-19 vaccines is the growing reluctance for otherwise healthy individuals to take vaccines. Tremendous progress has been made in the development of new vaccines, along with increasing access to new and underused vaccines in the lowest income countries. But vaccines, often lauded as one of the greatest public health interventions, are losing public confidence. Some vaccine experts describe the problem as a “crisis of public confidence” and a “vaccination backlash”. Public concerns about vaccine safety and vaccine legislation are as old as vaccines themselves, dating back to the anti-compulsory vaccination league against mandated smallpox vaccination in the mid-1800s. Some common concerns shared by the antivaccination groups of the 1800s and those of today are related primarily to arguments against mandated vaccination, or imposed vaccine schedules. But current antivaccination groups have new levels of global reach and influence, empowered by the internet and social networking capacities allowing like minds to rapidly self-organize transnationally, whether for or against vaccines. Many of these groups reach people who are not necessarily against vaccines, but who are seeking answers to questions about vaccine safety, vaccine schedules, changing policies, and the relevance of some new, and old, vaccines.

Traditional vaccines evoke concerns different from other health interventions because many healthy people need to be vaccinated to achieve a protective public health benefit. Several factors drive public questions and concerns: perceptions of business and financial motives of the vaccine industry and their perceived pressures on public institutions, such as during the H1N1 influenza response; coincidental rather than causal adverse events that are perceived as vaccine-related; challenges in management and communication of uncertainty about risks including serious, (albeit rare, ones); less risk tolerance for vaccines given to those who are healthy than for drugs given to treat an illness; skepticism of scientific truths, which later become untruths, or amended truths as new research becomes available; elitism of a group of people that believe they should not risk vaccination of their child if enough other children are being vaccinated; and/or outright nonacceptance of scientific evidence such as in the case of anti-vaccine movements that persist in the belief that autism can be caused by thiomersal or the measles.

For COVID-19 and many other diseases, the most affected population are individuals over the age of 60, who also represent the most difficult population to develop effective vaccines for. It is as-yet unclear what the long-term effectiveness of the present vaccines are for the elderly. As the immune system ages, the effectiveness and duration of vaccines wanes with it.

Coronaviruses make for difficult vaccine candidates because they produce many proteins that allow them to trick and evade the immune system. Coronaviruses (e.g., SARS-CoV-2) can play tricks with the immune system in a way other viruses cannot. The human immune system offers a two-pronged response to a viral invasion. One response produces antibodies which bind to the virus and eliminate the intruder. The other response more directly attacks infected cells. But coronaviruses (e.g., SARS-CoV-2) can mute the first response and make the other response hyperactive. Coronaviruses (e.g., SARS-CoV-2) effectively is amplifies what happens to humans naturally as our immune systems age. As a result, experiments with vaccines for Severe Acute Respiratory Syndrome (SARS) and Middle East Respiratory Syndrome (MERS) have not ended well. Some groups generated neutralizing antibodies, but they didn't provide adequate protection.

Globalization and human population growth have created pretty good ecosystems for new colonizing viruses. Every day the viral world makes trillions of random mutations and some of these mutations produce viruses that can adapt to human environments better than others

While the current emergency use authorization for COVID-19 vaccines includes individuals with many types of underlying medical conditions, currently there is no long-term safety data for persons with underlying medical conditions. Those individuals with underlying medical conditions are the most at risk from SARS-CoV-2. Underlying medicals conditions include, but are not limited to, asthma; reactive airways disease, or other chronic disorders of the pulmonary or cardiovascular systems; metabolic diseases such as diabetes, renal dysfunction, and hemoglobinopathies; or known or suspected immunodeficiency diseases or immunosuppressed states, such as HIV, The long-term effects on children or adolescents receiving aspirin or other salicylates of their association of Reye syndrome and salicylates with wild-type virus infections are also unknown. long-term effects on individuals with acute febrile illness are similarly unknown.

Transplant Recipients immunogenicity for persons with solid organ transplants varies according to transplant type. Among persons with kidney or heart transplants, the proportion that developed seroprotective antibody concentrations was similar or slightly reduced compared with healthy persons. However, a study among persons with liver transplants indicated reduced immunologic responses to the influenza vaccination, especially if vaccination occurred within the 4 months after the transplant procedure. There is also no known long-term safety data for transplant recipients who receive a COVID-19 vaccination.

In summary, it is clear that the current COVID-19 prevention and treatment guidelines are unsustainable in terms of human loss, economic impact, and burden on our healthcare system: the lack of pre-exposure or post exposure prophylaxis agents other than vaccines; the limited number of antiviral treatments recommended for COVID-19; the lack of any treatment for COVID-19 long haulers, and the uncertainties surrounding the safety, efficacy, and acceptance of the vaccines, clearly demonstrates an urgent ever pressing unmet need for new prophylaxis and accelerated treatment modalities.

### INCORPORATION BY REFERENCE

All publications, patents, and patent applications mentioned in this specification are herein incorporated by reference to the same extent as if each individual publication, patent, or patent application was specifically and individually indicated to be incorporated by reference. No discussion of any information within this application shall be construed as an admission of prior art.

### BRIEF DESCRIPTION OF THE DRAWINGS

The novel features of the are set forth with particularity in the appended claims. A better understanding of the features and advantages of the present will be obtained by reference to the following detailed description that sets forth illustrative embodiments, in which the principles of the are utilized, and the accompanying drawings of which:

FIG. 1 is a chart presenting COVID-19 viral reproduction versus various diseases.

FIG. 2 is a graph depicting viral reproduction versus percent of population required to be immune to disease to avoid a pandemic.

FIG. 3 provides a genomic organization of representative α, β, and γ coronaviruses illustrating the MHV genome along with the structural and accessory proteins in the 3′ regions of the HCoV-229E, Murine Hepatitis Virus (MHV), SARS-CoV, MERS, and Infectious Bronchitis Virus (IBV).

FIG. 4 is a summary of ACE2 expression in human tissues based on publicly available transciptomics and proteomics datasets.

FIG. 5 is a summary of ACE2 expression in human tissues based on publicly available transcriptomics and proteomics datasets

FIG. 6 is an illustration of SARS-CoV-2 spike protein binding to ACE2, viral entry, replication, and blocking of normal ACE2 function.

FIG. 7 is an illustration of the conversion of Angiotensin I into Angiotensin II along with ACE and ACE2 functionality.

FIG. 8 is an illustration of the glycosylation profile on coronavirus SARS-CoV-2.

FIG. 9 is a graph of the RNA tissues specificity for the ACE2 receptor, as outlined in the tissue atlas portion of the Human Protein Atlas.

FIG. 10 is a graph of the RNA expression overview for ACE2 receptor, as outlined in the tissue atlas portion of the Human Protein Atlas, Consensus Data Set.

FIG. 11 is an illustration of ACE2 RNA and protein expression summary in the female anatomy.

FIG. 12 is an illustration of ACE2 RNA and protein expression summary in the male anatomy.

FIG. 13 depicts the protein expression of Dipeptidyl Peptidase 4 (DDP4) as given by the Human Protein Atlas Consensus Data Set.

FIG. 14 depicts the protein expression of DPP4 dipeptidyl peptidase 4 concentration in the small intestine, colon, duodenum, liver, and kidneys.

FIG. 15 depicts the protein expression of CEACAMI as given by the Human Protein Atlas Consensus Data Set.

FIG. 16 depicts the RNA expression of CEACAM1 as given by the Human Protein Atlas Consensus Data Set.

FIG. 17 depicts the protein expression of APN as given by the Human Protein Atlas Consensus Data Set.

FIG. 18 depicts the RNA expression of APN as given by the Human Protein Atlas Consensus Data Set.

FIG. 19 depicts an Assay Plate Map for an experiment demonstrating the efficacy of CoGEN2 against SARS-CoV-2.

FIG. 20 presents the Cytoprotection of Vero E6 Cells against cell death after treatment with Pancreatic Enzyme Concentrate lot number 2226-0001.

FIG. 21 demonstrate cytotoxicity of Vero E6 Cells after treatment with Pancreatic Enzyme Concentrate lot number 2226-0001.

FIG. 22 demonstrate cytotoxicity of Vero E6 Cells against cell death after treatment with Pancreatic Enzyme Concentrate lot number 2226-0003.

FIG. 23 demonstrate cytotoxicity of Vero E6 Cells after treatment with Pancreatic Enzyme Concentrate lot number 2226-0003.

FIG. 24 demonstrate cytotoxicity of Vero E6 Cells against cell death after treatment with Pancreatic Enzyme Concentrate lot number 2226-0004.

FIG. 25 demonstrate cytotoxicity of Vero E6 Cells after treatment with Pancreatic Enzyme Concentrate lot number 2226-0003.

FIG. 26 demonstrate cytotoxicity of Vero E6 Cells against cell death after treatment with Microencapsulated Pancreatic Enzyme Concentrate.

FIG. 27 demonstrate cytotoxicity of Vero E6 Cells after treatment with Microencapsulated Pancreatic Enzyme Concentrate.

FIG. 28 demonstrate inhibition of SARS-CoV-2 in Vero E6 Cells with Multiple of Infection (MOI)=0.001 after treatment with Pancreatic Enzyme Concentrate lot number 2226-0001.

FIG. 29 demonstrate inhibition of SARS-CoV-2 in Vero E6 Cells with Multiple of Infection (MOI)=0.001 after treatment with Pancreatic Enzyme Concentrate lot number 2226-0003.

FIG. 30 demonstrate inhibition of SARS-CoV-2 in Vero E6 Cells with Multiple of Infection (MOI)=0.001 after treatment with Pancreatic Enzyme Concentrate lot number 2226-0004.

FIG. 31 demonstrate inhibition of SARS-CoV-2 in Vero E6 Cells with Multiple of Infection (MOI)=0.001 after treatment with Microencapsulated Pancreatic Enzyme Concentrate.

FIG. 32 demonstrate inhibition of SARS-CoV-2 in Vero E6 Cells with Multiple of Infection (MOI)=0.0001 after treatment with Pancreatic Enzyme Concentrate lot number 2226-0001

FIG. 33 demonstrate inhibition of SARS-CoV-2 in Vero E6 Cells with Multiple of Infection (MOI)=0.0001 after treatment with Pancreatic Enzyme Concentrate lot number 2226-0003.

FIG. 34 demonstrate inhibition of SARS-CoV-2 in Vero E6 Cells with Multiple of Infection (MOI)=0.0001 after treatment with Pancreatic Enzyme Concentrate lot number 2226-0004.

FIG. 35 demonstrate inhibition of SARS-CoV-2 in Vero E6 Cells with Multiple of Infection (MOI)=0.0001 after treatment with Microencapsulated Pancreatic Enzyme Concentrate.

FIG. 36 demonstrate inhibition of SARS-CoV-2 in Vero E6 Cells with Multiples of Infection (MOI)=0.0001 and MOI=0.001 after treatment with Pancreatic Enzyme Concentrate lot number 2226-0001.

FIG. 37 demonstrate inhibition of SARS-CoV-2 in Vero E6 Cells with Multiples of Infection (MOI)=0.0001 and MOI=0.001 after treatment with Pancreatic Enzyme Concentrate lot number 2226-0003.

FIG. 38 demonstrate inhibition of SARS-CoV-2 in Vero E6 Cells with Multiples of Infection (MOI)=0.0001 and MOI=0.001 after treatment with Sieved Pancreatic Enzyme Concentrate lot number 2226-0004.

FIG. 39 demonstrate inhibition of SARS-CoV-2 in Vero E6 Cells with Multiples of Infection (MOI)=0.0001 and MOI=0.001 after treatment with Microencapsulated Pancreatic Enzyme Concentrate.

FIG. 40 provides virus titer of SARS-CoV-2 in Vero E6 Cells with a Multiple of Infection (MOI)=0.001 after treatment with Sieved Pancreatic Enzyme Concentrate lot number 2226-0001.

FIG. 41 provides virus titer of SARS-CoV-2 in Vero E6 Cells with a Multiple of Infection (MOI)=0.0001 after treatment with Sieved Pancreatic Enzyme Concentrate lot number 2226-0001.

FIG. 42 provides virus titer of SARS-CoV-2 in Vero E6 Cells with a Multiple of Infection (MOI)=0.0001 after treatment with Microencapsulated Pancreatic Enzyme Concentrate.

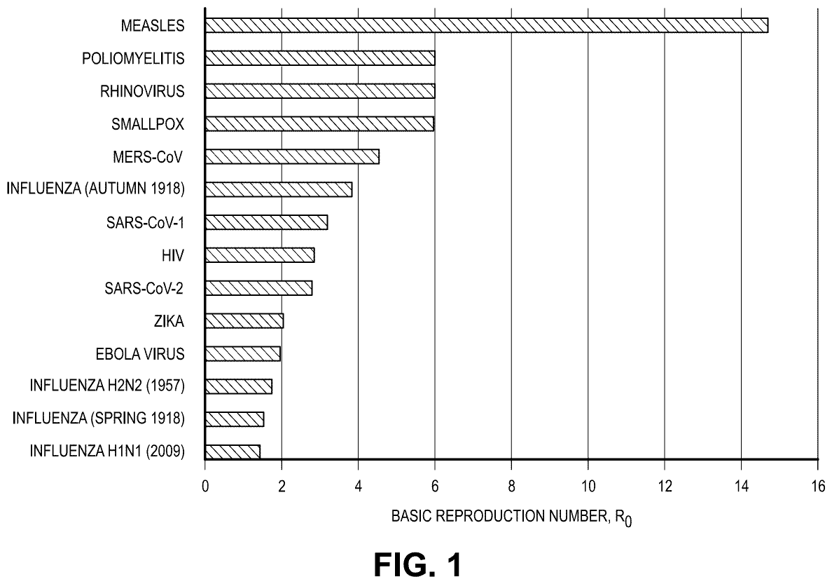

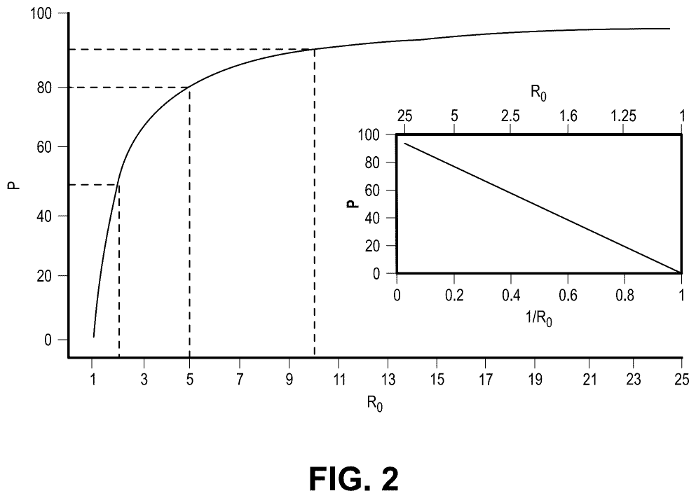

### DETAILED DESCRIPTION OF THE INVENTION

The present provides a method utilizing a pharmaceutical composition comprised of coated or uncoated digestive enzymes and their derivatives as a prophylaxis against, and treatment of a coronavirus infection (e.g., SARS-CoV-2 infection) resulting in disease (e.g., COVID-19).

### Coronaviruses Pre-SARS-CoV-2

Historically, coronaviruses (CoVs), enveloped positive-sense RNA viruses, are characterized by club-like spikes that project from their surface, an unusually large RNA genome, and a unique replication strategy. Coronaviruses cause a variety of diseases in mammals and birds ranging from enteritis in cows and pigs and upper respiratory disease in chickens to potentially lethal human respiratory infections. Coronaviruses (CoVs) are the largest group of viruses belonging to the Nidovirales order, which includes Coronaviridae, Arteriviridae, Mesoniviridae, and Roniviridae families. The Coronavirinae comprise one of two subfamilies in the Coronaviridae family, with the other being the Torovirinae. The Coronavirinae are further subdivided into four genera: the alpha (α), beta (β), gamma (γ), and delta (δ) coronaviruses. The viruses were initially sorted into these genera based on serology but are now divided by phylogenetic clustering.

All viruses in the Nidovirales order are enveloped, non-segmented positive-sense RNA viruses. They all contain very large genomes for RNA viruses, with some viruses having the largest identified RNA genomes, containing up to 33.5 kilobase (kb) genomes. Other common features within the Nidovirales order include: (1) a highly conserved genomic organization, with a large replicase gene preceding structural and accessory genes; (2) expression of many non-structural genes by ribosomal frameshifting; (3) several unique or unusual enzymatic activities encoded within the large replicase-transcriptase polyprotein; and (4) expression of downstream genes by synthesis of 3′ nested subgenomic mRNAs. In fact, the Nidovirales order name is derived from these nested 3′ mRNAs as nido is Latin for “nest.” The major differences within the Nidovirus families are in the number, type, and sizes of the structural proteins. These differences cause significant alterations in the structure and morphology of the nucleocapsids and virions.

### Genomic Organization

Coronaviruses contain a non-segmented, positive-sense RNA genome of ˜30 kb. The genome contains a 5′ cap structure along with a 3′ poly (A) tail, allowing it to act as an mRNA for translation of the replicase polyproteins. The replicase gene encoding the nonstructural proteins (nsps) occupies two-thirds of the genome, about 20 kb, as opposed to the structural and accessory proteins, which make up only about 10 kb of the viral genome. The 5′ end of the genome contains a leader sequence and untranslated region (UTR) that contains multiple stem loop structures required for RNA replication and transcription. Additionally, at the beginning of each structural or accessory gene are transcriptional regulatory sequences (TRSs) that are required for expression of each of these genes. The 3′ UTR also contains RNA structures required for replication and synthesis of viral RNA. The organization of the coronavirus genome is 5′-leader-UTR-replicase-S(Spike)-E (Envelope)-M (Membrane)-N (Nucleocapsid)-3′ UTR-poly (A) tail with accessory genes interspersed within the structural genes at the 3′ end of the genome. The accessory proteins are almost exclusively nonessential for replication in tissue culture; however, some have been shown to have important roles in viral pathogenesis.

### Virion Structure

Coronavirus virions are spherical with diameters of approximately 125 nm as depicted in recent studies by cryo-electron tomography and cryo-electron microscopy. The most prominent feature of coronaviruses is the club-shaped spike projections emanating from the surface of the virion. These spikes are a defining feature of the virion and give them the appearance of a solar corona, prompting the name, coronaviruses. Within the envelope of the virion is the nucleocapsid. Coronaviruses have helically symmetrical nucleocapsids, which is uncommon among positive-sense RNA viruses, but far more common for negative-sense RNA viruses.

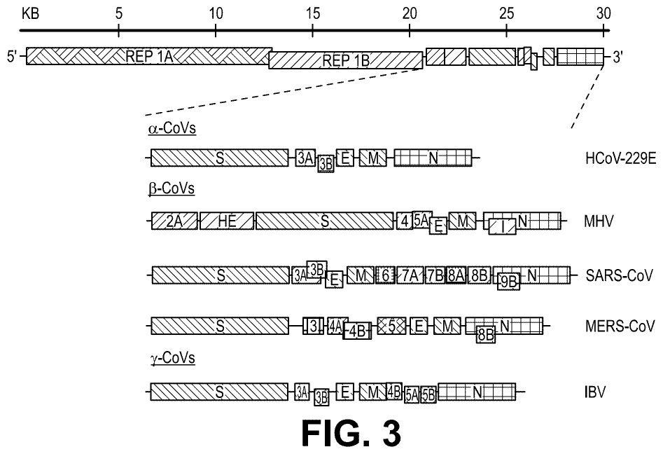

FIG. 3 provides a genomic organization of representative α, β, and γ coronaviruses illustrating the MHV genome along with the structural and accessory proteins in the 3′ regions of the HCoV-229E, MHV, SARS-CoV, MERS-CoV and IBV.

Coronavirus particles contain four main structural proteins. These are the spike (S), membrane (M), envelope (E), and nucleocapsid (N) proteins, all of which are encoded within the 3′ end of the viral genome. The S protein (˜150 kDa), utilizes an N-terminal signal sequence to gain access to the endoplasmic reticulum (ER), and is heavily N-linked glycosylated. Homotrimers of the virus encoded S protein make up the distinctive spike structure on the surface of the virus. The trimeric S glycoprotein is a class I fusion protein and mediates attachment to the host receptor. In most coronaviruses, the S protein is cleaved by a host cell furin-like protease into two separate polypeptides noted S1 and S2. S1 makes up the large receptor binding domain (RBD) of the S protein, while S2 forms the stalk of the spike molecule.

The M protein is the most abundant structural protein in the virion. It is a small (˜25-30 kDa) protein with three transmembrane domains and is thought to give the virion its shape. It has a small N-terminal glycosylated ectodomain and a much larger C-terminal end domain that extends 6-8 nm into the viral particle. Despite being co-translationally inserted in the ER membrane, most M proteins do not contain a signal sequence. Recent studies suggest the M protein exists as a dimer in the virion, and may adopt two different conformations, allowing it to promote membrane curvature as well as to bind to the nucleocapsid.

The E protein (˜8-12 kDa) is found in small quantities within the virion. The coronavirus E proteins are highly divergent but have a common architecture. The membrane topology of E protein is not completely resolved but most data suggest that it is a transmembrane protein. The E protein has an N-terminal ectodomain and a C-terminal endodomain and has ion channel activity. As opposed to other structural proteins, recombinant viruses lacking the E protein are not always lethal, although this is virus type dependent. The E protein facilitates assembly and release of the virus, but also has other functions. For instance, the ion channel activity in SARS-CoV E protein is not required for viral replication but is required for pathogenesis.

The N protein constitutes the only protein present in the nucleocapsid. It is composed of two separate domains, an N-terminal domain (NTD) and a C-terminal domain (CTD), both capable of binding RNA in vitro, but each domain uses different mechanisms to bind RNA. It has been suggested that optimal RNA binding requires contributions from both domains. N protein is also heavily phosphorylated, and phosphorylation has been suggested to trigger a structural change enhancing the affinity for viral versus non-viral RNA. The N protein binds the viral genome in a beads-on-a-string type conformation. Two specific RNA substrates have been identified for N protein: the TRSs and the genomic packaging signal. The genomic packaging signal has been found to bind specifically to the second, or C-terminal RNA binding domain. The N protein also binds nsp3, a key component of the replicase complex, and the M protein. These protein interactions likely help tether the viral genome to the replicase-transcriptase complex (RTC) and subsequently package the encapsidated genome into viral particles.

A fifth structural protein, the hemagglutinin-esterase (HE), is present in a subset of 3-coronaviruses. The protein acts as a hemagglutinin, binds sialic acids on surface glycoproteins, and contains acetyl-esterase activity. These activities are thought to enhance S protein-mediated cell entry and virus spread through the mucosa. Interestingly, HE enhances murine hepatitis virus (MHV) neurovirulence; however, it is selected against in tissue culture for unknown reasons.

### Viral Receptors

The initial attachment of the virion to the host cell is initiated by interactions between the S protein and its receptor. The sites of receptor binding domains (RBD) within the S1 region of a coronavirus S protein vary depending on the virus, with some having the RBD at the N-terminus of Si (e.g., MHV), while others (e.g., SARS-CoV), have the RBD at the C-terminus of S1. The S protein-receptor interaction is the primary determinant for a coronavirus to infect a host species and also governs the tissue tropism of the virus. Many coronaviruses utilize peptidases as their cellular receptor. It is unclear why peptidases are used, as entry occurs even in the absence of the enzymatic domain of these proteins. Many α-coronaviruses utilize aminopeptidase N (APN) as their receptor, SARS-CoV and HCoV-NL63 use angiotensin converting enzyme 2 (ACE2) as their receptor, MHV enters through CEACAM1, and the recently identified MERS-CoV binds to dipeptidyl-peptidase 4 (DPP4) to gain entry into human cells. Table 1 below presents a listing of known CoV receptors pre-SARS-CoV-19.

TABLE 1
Virus Receptor References

| | Receptor |
|---|---|
| Alphacoronaviruses | |
| HCoV-229E | Aminopeptidase N (APN) |
| HCoV-NL63 | Angiotensin Converting Enzyme 2 (ACE2) |
| TGEV | APN |
| PEDV | APN |
| FIPV | APN |
| CCoV | APN |
| Betacoronaviruses | |
| MHV | Murine Carcinoembryonic Antigen-Related |
| | Adhesion Molecule I (mCEACAM) |
| BCoV | N-acetyl-9-O-acetylneuraminic acid |
| SARS-CoV | ACE2 |
| MERS-CoV | Dipeptidyl Peptidase 4 (DPP4) |

### SARS-CoV-2 Phylogenetics and Taxonomy

SARS-CoV-2 also belongs to the broad family of viruses known as coronaviruses. It is a positive-sense single-stranded RNA (+ssRNA) virus, with a single linear RNA segment. Other coronaviruses are capable of causing illnesses ranging from the common cold to more severe diseases such as MERS (fatality rate ˜34%). It is the seventh known coronavirus to infect people, after 229E, NL63, OC43, HKU1, MERS-CoV, and the original SARS-CoV. The SARS-CoV-2 virus belongs to the Realm: Riboviria, Kingdom: Orthornavirae, Phylum: Pisuviricota, Class: Pisoniviricetes, Order: Nidovirales, Family: Coronaviridae, Genus: Betacoronavirus, Subgenus: Sarbecovirus, Species Severe acute Respiratory syndrome-related coronavirus, Strain: Severe acute respiratory syndrome coronavirus 2 (SARS-Cov-2).

Like the SARS-related coronavirus strain implicated in the 2003 SARS outbreak, SARS-CoV-2 is a member of the subgenus Sarbecovirus (beta-CoV lineage B). Its RNA sequence is approximately 30,000 bases in length. SARS-CoV-2 is unique among known betacoronaviruses in its incorporation of a polybasic cleavage site, a characteristic known to increase pathogenicity and transmissibility in other viruses.

Proteolytic excision at polybasic cleavage sites is a post-translational modification phenomenon required for the maturation and activation of several precursor proteins, including neuropeptides, peptide hormones, growth factors, membrane receptors, coagulation factors, and adhesion proteins.

Various cellular proteases such as furin, trypsin, cathepsin, and trans-membrane protease/serine that catalyze the proteolytic activation process cleave various viral cell surface proteins, which is required for the viral entry to host cells. A wide range of evidence indicates that the proteolytic cleavage of viral surface proteins at the polybasic cleavage site is essential for viral pathogenicity, virulence, and interspecies transmission.

The pathogenicity and interspecies transmission of novel coronavirus (SARS-Cov-2) strictly depends on the S protein present on the viral cell surface. The S protein plays a vital role in attaching the virus with the host cell receptor (e.g., ACE2) and subsequently mediating viral entry through membrane fusion. Proteolytic cleavage of the S protein is an indispensable step for the viral entry to host cells. Many host cellular proteases, including furin, trypsin, and cathepsin, are present to catalyze the proteolytic activation of the S protein. Of these proteases, furin recognizes a polybasic cleavage site and cleaves the S protein during its synthesis in the trans-Golgi network or during entry of the virus in endosomes. In contrast, trypsin cleaves the S protein at monobasic cleavage sites in the extracellular area and the cell surface. Most interestingly, the acquisition of a polybasic cleavage site at the junction of two domains of the spike protein (S1 and S2) is a newly evolved feature of a novel coronavirus, which may be a potential reason for the deadly outbreak of this highly pathogenic virus. The polybasic cleavage site is generated as a result of the insertion of 12 nucleotides, which subsequently results in the predicted acquisition of 3 glycans around the site. This particular feature is not present in the spike proteins of other coronaviruses, including bat coronavirus and SARS-CoV.

Each SARS-CoV-2 virion is 50-200 nanometers in diameter. Protein modeling experiments on the S protein of the virus soon suggested that SARS-CoV-2 has sufficient affinity to the receptor angiotensin converting enzyme 2 (ACE2) on human cells to use them as a mechanism of cell entry. SARS-CoV-2 has a higher affinity to human ACE2 than the original SARS virus strain. SARS-CoV-2 may also assist in cell entry. Initial S protein priming by transmembrane protease, serine 2 (TMPRSS2) is essential for entry of SARS-CoV-2. After a SARS-CoV-2 virion attaches to a target cell, the cell's protease TMPRSS2 cuts open the S protein of the virus, exposing a fusion peptide in the S2 subunit, and the host receptor ACE2. After fusion, an endosome forms around the virion, separating it from the rest of the host cell. The virion escapes when the pH of the endosome drops or when cathepsin, a host cysteine protease, cleaves it. The virion then releases RNA into the cell and forces the cell to produce and disseminate copies of the virus, which infect more cells. SARS-CoV-2 produces at least three virulence factors that promote shedding of new virions from host cells and inhibit immune response. Whether they include downregulation of ACE2, as seen in similar coronaviruses remains under investigation.

### COVID-19 Pathogenesis

The pathogenesis of COVID-19 has a number of significant attributes. For example, not all people exposed to SARS-CoV-2 are infected and not all infected patients develop severe respiratory illness. SARS-CoV-2 infection can be roughly divided into three stages: stage I, an asymptomatic incubation period with or without detectable virus; stage II, non-severe symptomatic period with the presence of virus; and stage III, severe respiratory symptomatic stage with high viral load. From the point of view of prevention, individuals at stage I (the stealth carriers) are the least manageable because, at least on some occasions, they unknowingly spread the virus.

Clinically, the immune responses induced by SARS-CoV-2 infection are two-phased. During the incubation and non-severe stages, a specific adaptive immune response is required to eliminate the virus and to preclude disease progression to severe stages. Genetic differences are well-known to contribute to individual variations in the immune response to pathogens. However, when a protective immune response is impaired, virus will propagate and massive destruction of the affected tissues will occur, especially in organs that have high ACE2 expression, such as intestine and kidney. The damaged cells induce innate inflammation in the lungs that is largely mediated by pro-inflammatory macrophages and granulocytes. Lung inflammation is the main cause of life-threatening respiratory disorders at the severe stage.

Alarmingly, after discharge from hospital, some patients remain/return viral positive and others even relapse. This indicates that a virus-eliminating immune response to SARS-CoV-2 may be difficult to induce, at least in some patients, and vaccines may not work in these individuals.

It has become increasingly clear that the mechanism for SARS-CoV-2 infection is the requisite binding of the virus to the membrane-bound form of angiotensin-converting enzyme 2 (ACE2) and subsequent internalization of the complex by the host cell. As ACE2 may be a coreceptor for the coronavirus, new therapeutic approaches are being researched to block the enzyme or reduce its expression to prevent the cellular entry and subsequent SARS-CoV-2 infection in tissues that express ACE2 including lung, heart, kidney, brain, and gut. The blocking on the host side or on the viral side are both being given consideration as part of the novel paradigms being considered both for anti-virial medications and vaccine development.

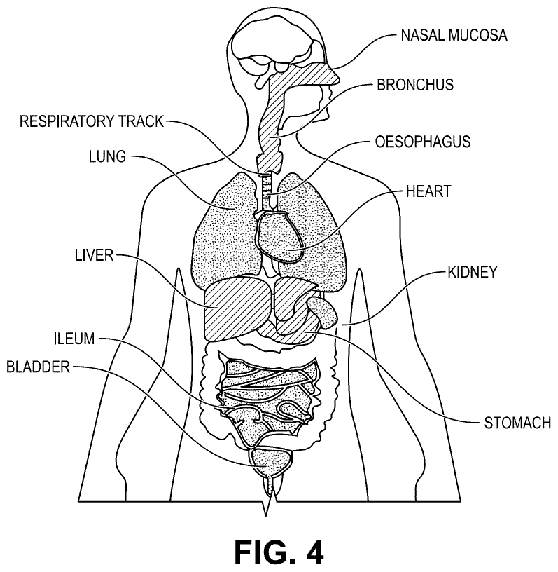

By examining ACE2 expression pattern in different organs, tissue, and cells, we can determine areas of risk to SARS-CoV-2 virus infection via the ACE2 surface enzyme as target cells expressing ACE2 permit coronavirus entry, multiplication, spread, and pathogenesis. FIG. 4 presents a map of ACE2 expression over different cell types of different organs to predict the risk for SARS-CoV-2 virus infection and injury. In addition to the known distribution of ACE2 in the lungs and kidneys, FIG. 4 indicates that there are significant levels of ACE2 in the heart with the highest levels of expression being found in the small intestines, especially the ileum.

Based upon stringently-validated immunohistochemical analysis and high-throughput mRNA sequencing from several datasets, it has been shown that ACE2 expression is mainly localized to microvilli of the intestinal tract as well as renal proximal tubules, gallbladder epithelium, testicular Sertoli cells and Leydig cells, glandular cells of seminal vesicle, and cardiomyocytes. The expression in several other previously reported locations, including alveolar type II (AT2) cells, could not be confirmed. Furthermore, ACE2 expression was absent in the AT2 lung carcinoma cell line A549, often used as a model for viral replication studies.

Through the use of two independent, well-characterized antibodies for immunohistochemical analysis of ACE2 and stringent validation criteria and comparison of the protein expression profiles with multiple transcriptomics datasets, reliable expression could only be confirmed in microvilli of the intestinal tract and renal proximal tubules, in gallbladder epithelium, testicular Sertoli cells and Leydig cells, a subset of glandular cells in seminal vesicle and in cardiomyocytes, with no detectable expression in lung or respiratory epithelia.

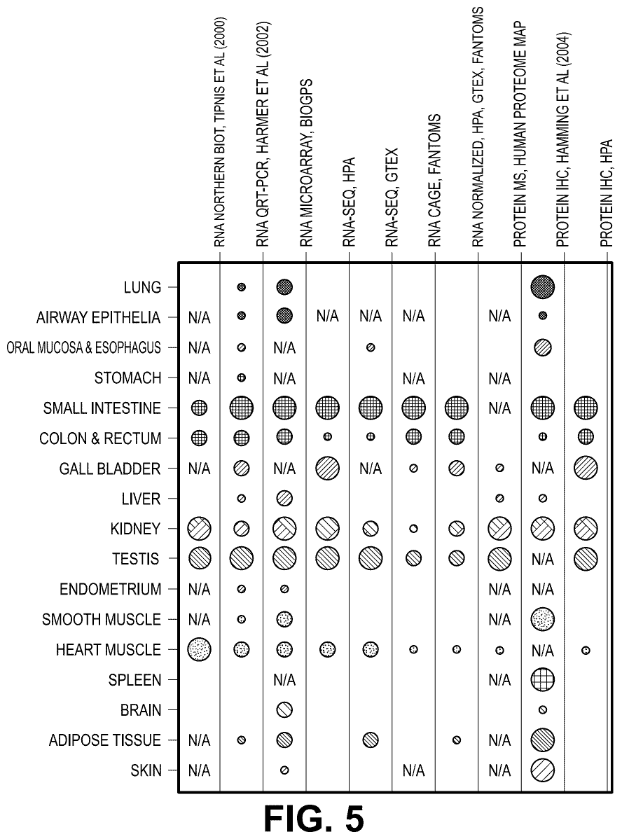

FIG. 5 presents a summary of ACE2 expression in human tissues based on publicly available transcriptomics and proteomics datasets. As shown three different sizes of circles, large, medium, and small, represent high, medium, or low expression levels, respectively. The crosshatch in each circle represents an organ system. A consistent expression in the intestinal tract, gallbladder, kidney, testis, and heart muscle is observed across all datasets. Note that the broadest reported expression profile includes lung, oral mucosa, esophagus, spleen, adipose tissue, smooth muscle, brain, and skin. N/A indicates that no data is available.

All studied datasets confirm a consistent high expression in the intestinal tract, gallbladder, and kidney. In addition to respiratory symptoms, the closely related SARS-CoV that caused the SARS outbreak was shown to also cause diarrhea, impaired liver function, and elevation of non-cardiac creatine kinase, suggesting tropism of the virus to other organs well in line with the tissues showing the highest expression levels of ACE2. Interestingly, in a recent study on pediatric COVID-19 individuals, 8 out of 10 cases showed rectal swabs positive for SARS-CoV-2 virus, suggesting that the gastrointestinal tract may shed virus, and that fecal-oral transmission may be a possible route for infection.

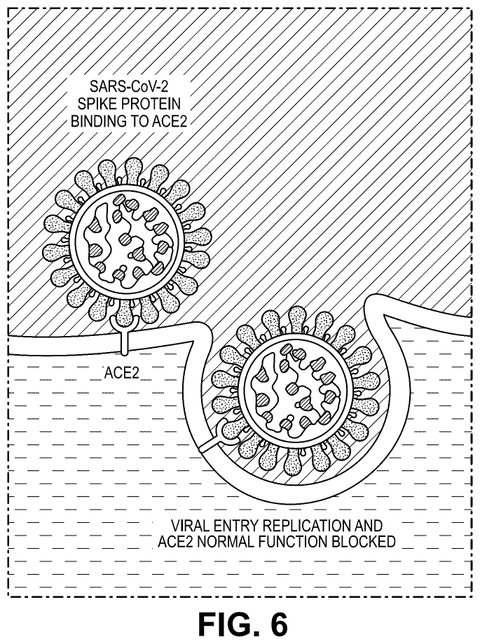

The intracellular entry by SARS-CoV-2 and SARS-CoV, appears to be governed by the spike protein (S Protein). This protein binds via a receptor-binding region to the extracellular domain of ACE2 with high affinity of 15 nM. Cleavage of the S protein along dibasic arginine sites by the host protease TMPRSS2 to generate the S1 and S2 subunits is a critical step for S2-induced membrane fusion and viral internalization by endocytosis with ACE2 in the pulmonary epithelium. FIG. 6 is an illustration of SARS-CoV-2 S protein binding to ACE2, viral entry, replication, and blocking of normal ACE2 function.

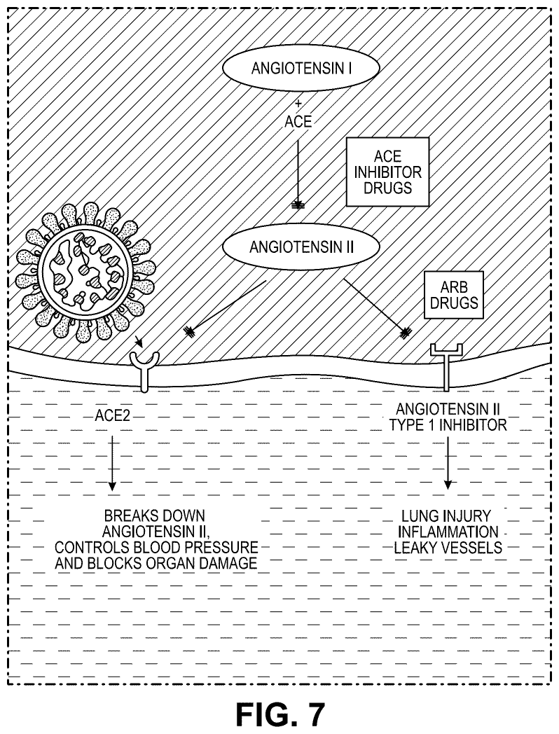

The ACE enzyme converts angiotensin I into angiotensin II. The main role of ACE2 is to break down angiotensin II into molecules that counteract angiotensin II's harmful effects; but if the virus occupies the ACE2 ‘receptor’ on the surface of cells, then its role is blunted. Drugs called ACE inhibitors inhibit the formation of angiotensin II, which would otherwise interact with the angiotensin type 1 receptor to produce tissue damage and inflammation. Drugs called Angiotensin II receptor blockers (ARBs) block angiotensin II from interacting with its receptor. FIG. 7 depicts the conversion of angiotensin I into angiotensin II along with ACE and ACE2 functionality.

In the association between angiotensin converting enzyme 2 (ACE2) and SARS-CoV-2, ACE2 has been shown to be a co-receptor for viral entry for SARS-CoV-2 with increasing evidence that it has a protracted role in the pathogenesis of COVID-19. ACE2 has a broad expression pattern in the human body with strong expression as noted earlier in the gastrointestinal system, heart, and kidney with more recent data identifying expression of ACE2 in type II alveolar cells in the lungs. The concern that Angiotensin-converting enzyme inhibitors (ACEIs) and Angiotensin II receptor blockers (ARBs) affect the severity and mortality of COVID-19 is 2-fold. When the SARS-CoV-2 virus enters the target cell, a surface unit of the S glycoprotein binds to a cellular receptor. Upon entry, cellular proteases cleave the S protein which leads to fusion of the viral and cellular membranes. SARS-CoV has previously been shown to enter the cell via the ACE2 receptor, primed by the cellular serine protease TMPRSS2, and recent studies suggest that also SARS-CoV-2 employs ACE2 and TMPRSS2 for host cell entry.

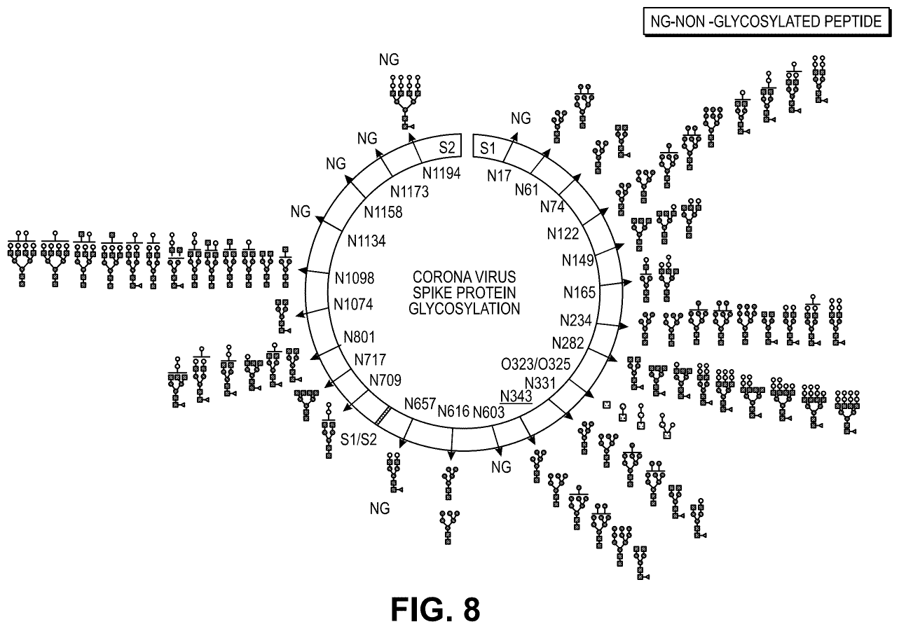

The S proteins of SARS-CoV-2 are heavily glycosylated. This S protein, which facilitates viral attachment, entry and membrane fusion, plays a critical role in the elicitation of the host immune response. Viral glycosylation is a mechanism viruses and other obligate parasites use to camouflage their presence and hinder immune response by the body. These glycans of the S protein have been shown to protect a large portion of the S protein from immune recognition, with the exception of the ACE2 binding region. The glycan coated S protein is considered a trimeric class I fusion protein that is composed of two functional subunits responsible for receptor binding, (SIsubunit) and membrane fusion (S2 subunit), together possessing 22 potential N-glycosylation sites. This form of glycan binding to a protein through the nitrogen region of amino acids such as asparagine, is common to many viruses and occurs early during the synthesis of the viral proteins. Alternatively, post-translational modifications in the endoplasmic reticulum can also lead to O-glycosylation, for example, attachment of the carbohydrate through the oxygen of an amino acid. Various studies have produced slightly differing results with respect to the level of glycosylation on the S protein. One study utilizing high-resolution LC-MS/MS found 17 N-glycosylation sites occupied out of 22 potential sites along with two O-glycosylation sites bearing core-1 type O-glycans. Some N-glycosylation sites were partially glycosylated along with a high level of mannose and complex glycosylation along with O-glycans on the receptor binding domain of the S1 subunit of the S protein. In another study, all 22 sites are occupied most of the time while another study found glycans at all 22 sites. FIG. 8 illustrates the glycosylation profile on SARS-CoV-2.

It has further been demonstrated that the most accessible portion of the S protein is at the level of the ACE2 binding domain. To date, there do not appear to be any mutations to the N-linked glycosylation sites in SARS-CoV-2, especially compared to other viruses, such as in the HIV-1 envelope. This stable configuration of the glycosylated sites makes the S protein a target for blockage or destruction.

Site-specific mass spectrometric examination of the glycan structure in vitro on a recombinant SARS-CoV-2 S immunogen employed enzymatic degradation of the S protein to examine the glycan subunits located on each portion of the S protein. The site-specific glycosylation suggests that the glycan shield of SARS-CoV-2 S protein is consistent with other coronaviruses and similarly exhibits numerous vulnerabilities throughout the glycan shield. Trace levels of O-linked glycosylation at T323/5325 with over 99% of these sites unmodified suggesting that O-linked glycosylation of this region is minimal when the structure is native-like. The predominance of N-glycosylation represents an attack site for unmasking of the S protein.

### Gastro-Intestinal Infectivity

The disease COVID-19 has been characterized as a novel acute respiratory syndrome caused by SARS CoV-2 virus, which is a highly transmissible infectious disease. The S protein of the SARs-CoV-2 virus is responsible for the infectivity with the S1 subunit responsible for the attachment of the virus and the S2 subunit responsible for the fusion of the viral and the human membranes. The area of entry of the virus and subsequent attachment is the ACE2 receptor, with a high affinity for binding attachment. Both anti-viral treatments and vaccines being developed appear, in many cases, to target the S protein to essentially try and neutralize it, thus, preventing binding to or a targeting the ACE2 entry receptor.

The primary route of infectivity of the SARS-CoV-2 virus is believed to be through the respiratory tract. The GI tract is also a major location for ACE2 receptors. The major expression of the ACE2 receptors in the GI tract allows the gut acts as a major portal of entry and potentially gestation of the SARS-CoV-2 virus in humans. By way of example, in the case of a mother and infant both of whom were affected with SARS-CoV-2, both the neonate (<28 days) and her mother expressed viral loads in the stool, with the viral loads remaining extremely high in both well past symptom amelioration. It was further noted that the stool samples could be positive for SARS-CoV-2 irrespective of the presence of gastrointestinal symptoms could remain positive for a least a month. Further, the SARS-CoV-2 virus was present early in the stools prior to the onset of symptoms.

Digestive symptoms occur in patients with SARS-CoV-2 infection. In April of 2020, the results of a descriptive cross-sectional multi-center study of 204 patients who presented in three hospitals in China between January and February of 2020 became available. Of the 204 patients, 103 reported digestive symptoms including diarrhea and vomiting. As the severity of the disease increased, the digestive symptoms became more pronounced. Patients with digestive symptoms had a significantly longer time from the onset to admission than patients without digestive symptoms (9.0 days vs. 7.3 days). Patients with digestive symptoms had higher mean liver enzyme levels, lower monocyte count, longer prothrombin time, and received more antimicrobial treatments than those without digestive symptoms.

While the current thinking about SARS-CoV-2 infection is that the primary site of infectivity is the lungs, and that the virus is inhaled into the body, there exists emerging evidence that the virus could enter through a fecal oral route, and or that viral shedding could become a source of re-infection in the general population.

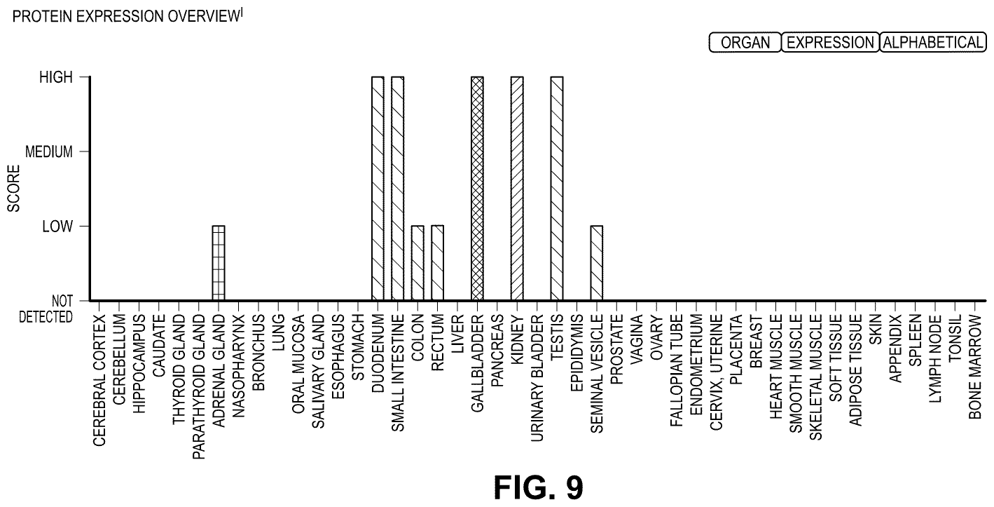

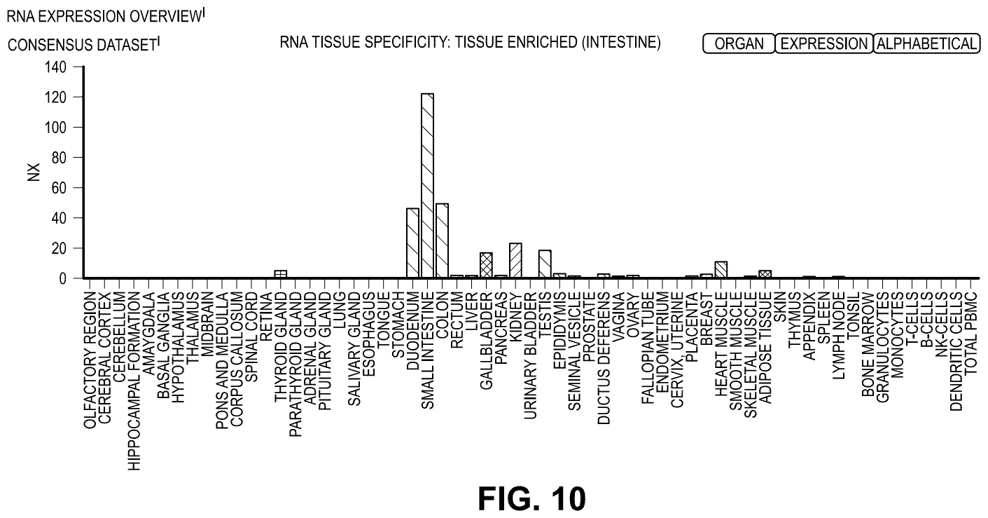

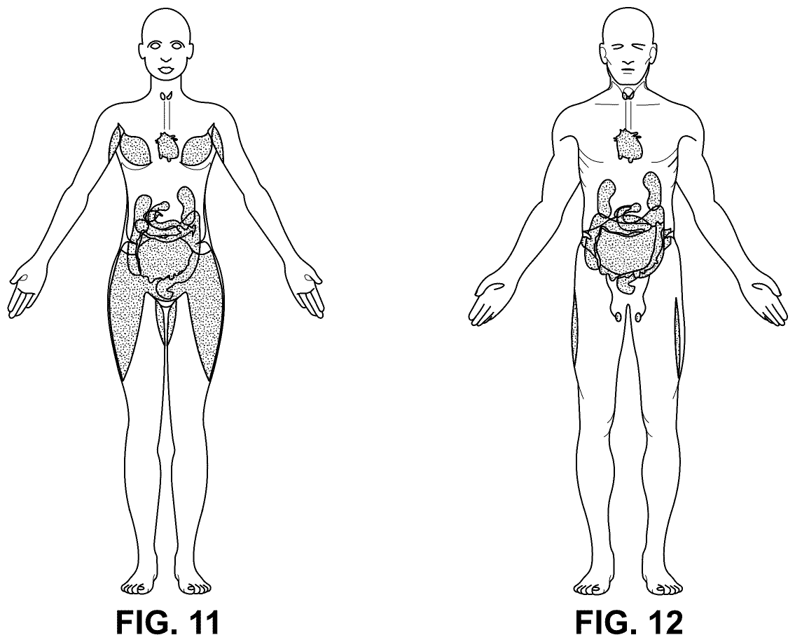

RNA tissue specificity for the ACE2 receptor is outlined in the tissue atlas portion of the Human Protein Atlas. FIG. 9 is a graph of the RNA tissues specificity for the ACE2 receptor, as outlined in the tissue atlas portion of the Human Protein Atlas and FIG. 10 is a graph of the RNA expression overview for ACE2 receptor, as outlined in the tissue atlas portion of the Human Protein Atlas, Consensus Data Set. FIG. 11 is an illustration of ACE2 RNA and protein expression summary in the female anatomy while FIG. 12 is an illustration of ACE2 RNA and protein expression summary in the male anatomy.

### Vaccines and Anti-Viral Treatments

The need to target coronaviruses (e.g., SARS-CoV-2) prior to infectivity would be optimal, thereby keeping infectivity rates lower and targeting the early stage of the disease. Vaccines are the most common form of mass control of infectious viral disease. There are, however, at least three major impediments to vaccine development for coronaviruses (e.g., SARS-CoV-2). First, the S protein is a promising immunogen but being able to optimize the design by targeting a yet to be identified portion of the of the S protein. Second, earlier work with the development of SARS and MERS vaccine raised concerns about the exacerbation of lung disease depending upon the dose administered. Third, the course of and duration of natural immunity is not yet understood. Inferring the course and duration of natural immunity from SARS and MERS disease is an uncertain proposition.

### Enzymatic Anti-Viral Treatment

The present application is directed to enzyme compositions and methods for treating coronaviruses. In some cases, treatment is of subjects diagnosed with a coronavirus infection. In other cases, treatment is prophylactic of, for example, essential workers who are at higher risk for infection.

One aspect of enzymatic anti-viral treatment provided herein relates to compositions that comprise one or more enzymes that target the S protein (e.g., the S1 subunit) of a coronavirus (e.g., SARS-CoV-2), thereby rendering the virus inactive and reducing or eliminating the possibility of infectivity. Enzymatic unmasking of viral surface glycans renders the S protein vulnerable to enzymatic protease degradation, also reducing or elimination the possibility of infectivity. Delivery of one or more enzymes that target the S protein (e.g., the S1 subunit) renders the virus inactive and reduces viral loading, thereby aiding in alleviating the symptoms of a coronavirus infection (e.g., COVID-19) and aiding in patient recovery. Prophylactically, delivery of one or more enzymes that target the S protein (e.g., the S1 subunit) to a subject prevents that subject from being infected.

One composition described herein comprises uncoated enzymes. Another composition described herein comprises coated enzymes. Coated and uncoated enzymes are able to overcome the challenges associated with highly targeted immunological or pharmacological functional interference of the S protein by attacking at both the level of the S protein itself as well as the glycan mask covering a large part of the S protein.

In one embodiment, a composition to be administered to an infected subject for treatment or prophylaxis contains a coated enzymatic core, where the core has high levels of proteases, as well as amylases and lipases, and which core provides for attack of the S protein by several proteases as well as attack of the glycoprotein mask by the amylases, individually or jointly reducing the infectivity of a coronavirus (e.g., SARS-CoV-2).

Utilizing the proteases and amylases to attack vulnerable portions of the S protein will significantly impede or eliminate the proper functioning of the S protein, thereby reducing or neutralizing the ability of the virus to infect human cells. Proteases can attack multiple sites of on the S protein.

It should be noted that the embodiments described herein are not limited to SARS-CoV-2 and any resultant infection resulting in COVID-19 but are also applicable to other coronaviruses described herein. In one non-limiting aspect, provided herein is an oral preparation (e.g., a pharmaceutical composition) comprising coated or uncoated enzymes which is specifically formulated to allow passage through the oropharynx with stomach targeted delivery to the early portion of the small intestines, where there is a high prevalence of ACE2 receptors. In another aspect, provided herein is a parenteral preparation comprising coated or uncoated enzymes. In another aspect, provided herein is a nasal preparation comprising coated or uncoated enzymes. In another aspect, provided herein is a nasal preparation comprising coated or uncoated enzymes. In another aspect, provided herein is a preparation for percutaneous endoscopic gastrostomy (PEG), esophagogastroduodenoscopy (EGD), or gastrostomy (G-tube) insertion comprising coated or uncoated enzymes. In another aspect, provided herein is a suppository comprising coated or uncoated enzymes.

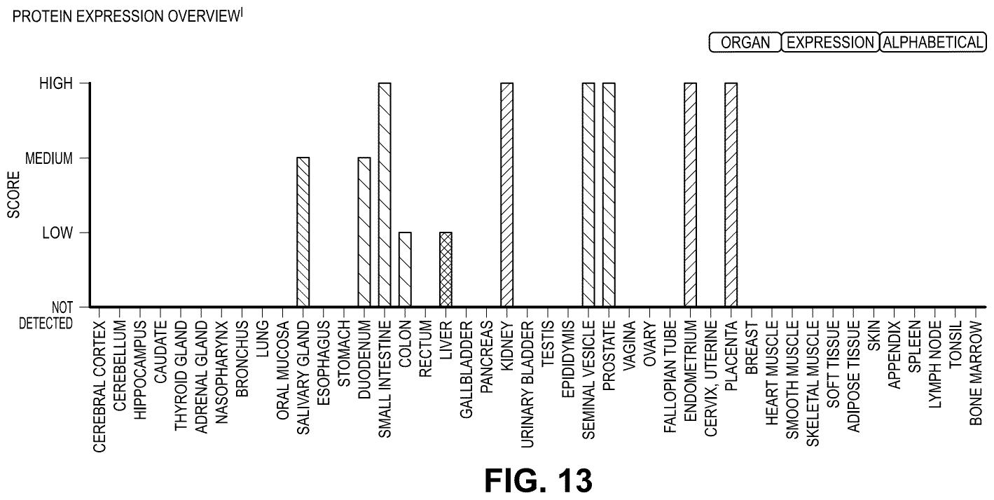

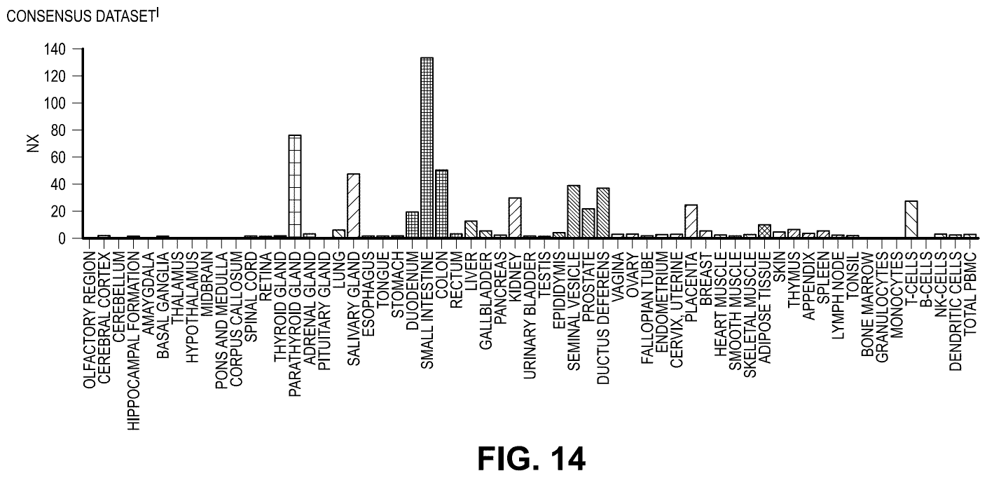

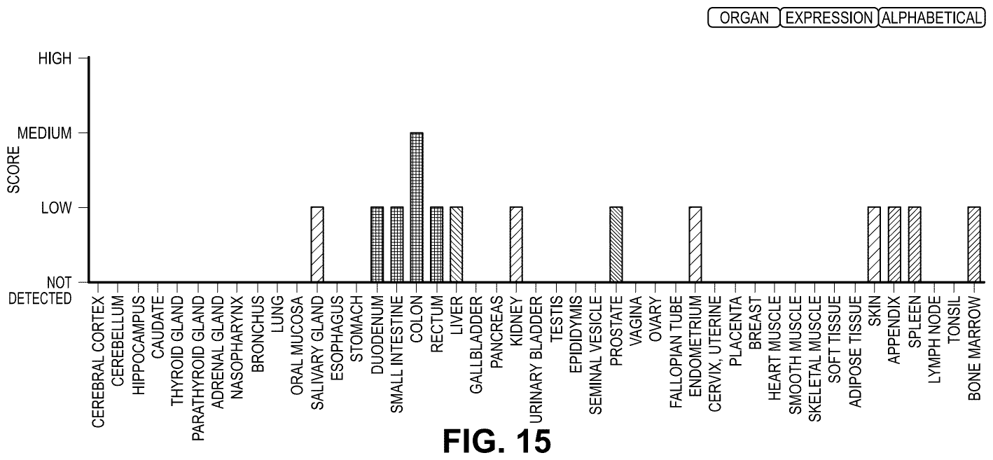

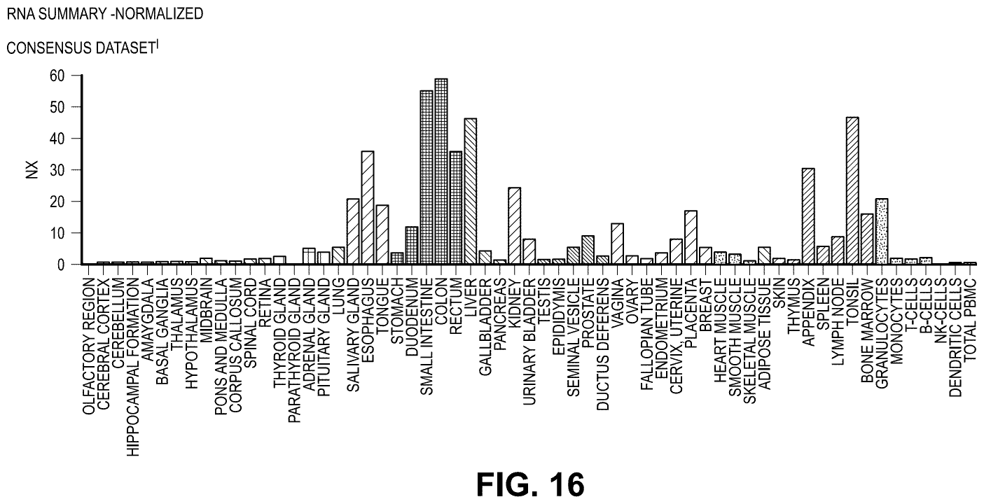

As previously discussed, the viral attachment or entry point for various coronaviruses includes ACE2, along with APN, mCEACAM, and/or DPP4. FIG. 13 depicts the protein expression of DDP4 as given by the Human Protein Atlas Consensus Data Set. Note the concentration of DDP4 expression in the small intestine, colon, and duodenum. In addition, there are high degrees of expression in the liver and kidneys, which are sites of tissue damage in subjects infected with a coronavirus (e.g., SARS-CoV-2). FIG. 14, also from the Human Protein Atlas, shows a similar concentration of DPP4 dipeptidyl peptidase 4 concentration in the small intestine, colon, and duodenum, along with liver, and kidneys. FIG. 15 depicts the protein expression of CEACAM1 as given by the Human Protein Atlas Consensus Data Set. Note the similar concentration of CEACAM1 expression in the small intestine, colon, and duodenum. In addition, there are high degrees of expression in the rectum, liver, kidneys, and prostate, which are sites of tissue damage in subjects infected with a coronavirus (e.g., SARS-CoV-2). FIG. 16 depicts the RNA expression of CEACAM1 as given by the Human Protein Atlas Consensus Data Set. Note the similar concentration of CEACAM1 Expression in the small intestine, colon, and duodenum. In addition, there are high degrees of expression in the rectum, liver, kidneys, and prostate.

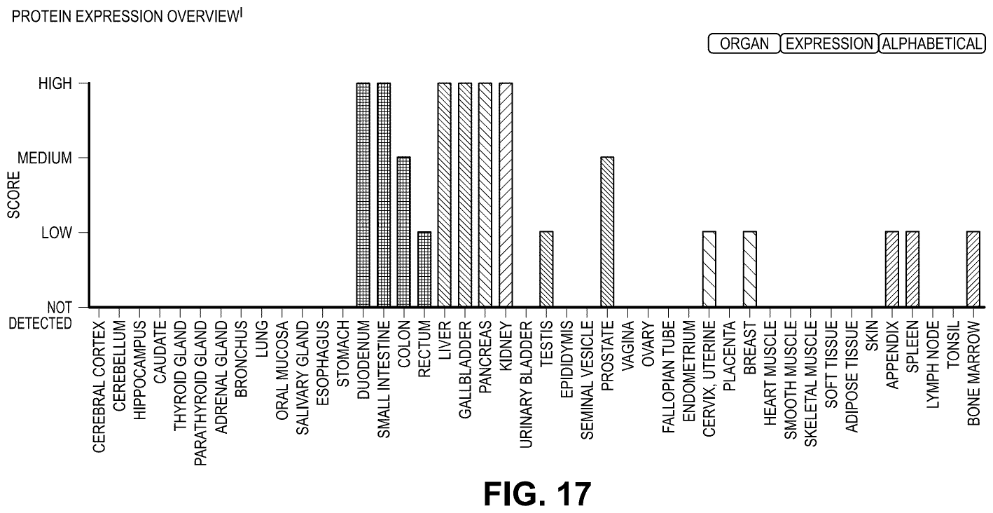

APN is also known as Membrane Alanyl Aminopeptidase and as Alanyl Aminopeptidase (AAP). Aminopeptidase N(APN) is an enzyme that in humans is encoded by the ANPEP gene. FIG. 17 depicts the protein expression of APN as given by the Human Protein Atlas Consensus Data Set. Note the similar concentration of APN Expression in the duodenum, small intestine, colon, and rectum. In addition, there are high degrees of expression in the liver, kidneys, gall bladder, pancreas, and prostate, which are sites of tissue damage in subjects infected with a coronavirus (e.g., SARS-CoV-2). FIG. 18 depicts the RNA expression of APN as given by the Human Protein Atlas Consensus Data Set. Note the similar concentration of APN expression in the duodenum, small intestine, colon, and rectum. In addition, there are high degrees of expression in the liver, kidneys, gall bladder, pancreas, and prostate, which are sites of tissue damage in subjects infected with a coronavirus (e.g., SARS-CoV-2).

In one embodiment, an encapsulated or coated enzymatic core that contains high levels of one or more protease(s), one or more lipase(s), one or more amylase(s), or a combination thereof provides for attack of the S protein by the one or more protease(s), as well as attack of the glycoprotein mask by the one or more amylase(s), individually or jointly affect the infectivity of a coronavirus (e.g., SARS-CoV-2).

In one embodiment, coated or uncoated digestive enzymes and their derivatives are utilized to reduce the transmission rate of a coronavirus including, but not limited to, infections from SARS-CoV-2. In another embodiment, coated or uncoated digestive enzymes and their derivatives may be utilized as a pre- and or post-exposure prophylaxis against, and the treatment of, coronavirus infections including, but not limited to, a Coronavirus HCoV-229E, a Human Coronavirus HCoV-NL63, a Transmissible Gastroenteritis Virus (TGEV), a Porcine Epidemic Diarrhea Virus (PEDV), a Feline Infectious Peritonitis Virus (FIPV), a Canine Coronavirus (CCoV), a Murine Hepatitis Virus (MHV), a Bovine Coronavirus (BCoV), a Severe Acute Respiratory Syndrome Coronavirus (SARS-CoV-2), a SARS-CoV-1 (SARS1), a Middle East Respiratory Syndrome Coronavirus (MERS-CoV), or a combination thereof.

In one embodiment, coated or uncoated digestive enzymes are utilized to alleviate the symptoms of a coronavirus infection (e.g., a SARS-CoV-2 infection). A method of treating a coronavirus infection comprises administering to a subject one or more digestive enzyme(s) (either naturally-derived, recombinantly-derived, or their derivatives) in an amount effective to reduce the symptoms of a coronavirus infection (e.g., reduce symptoms of COVID-19). Administration can be via any suitable route including, but not limited to, oral, nasal, parenteral, percutaneous endoscopic gastrostomy (PEG), intravenous, esophagogastroduodenoscopy (EGD), gastrostomy (G-tube) insertion, rectal, etc.

Provided herein is a method for treating a subject exhibiting one or more symptoms of coronavirus infection (e.g., COVID-19) in a subject in need thereof, comprising administering to the subject a therapeutically effective amount of a composition comprising coated or uncoated digestive enzymes. In one embodiment, the one or more symptoms of the coronavirus infection include, but are not limited to, one or more of fever, chills, cough, sore throat, fatigue, malaise, headache, muscle pain, body aches, shortness of breath, dyspnea, loss of taste, loss of smell, gastrointestinal symptoms (including, but not limited to nausea, vomiting, and diarrhea), or a combination thereof. In one embodiment, the one or more symptoms of COVID-19 include, but are not limited to, one or more of fever, chills, cough, sore throat, fatigue, malaise, headache, muscle pain, body aches, shortness of breath, dyspnea, loss of taste, loss of smell, gastrointestinal symptoms (including, but not limited to nausea, vomiting, and diarrhea), or a combination thereof.

Provided herein is a method for diagnosing and treating a subject that is asymptomatic for a coronavirus, comprising diagnosing the subject with a coronavirus infection and then treating the subject by administering a therapeutically effective amount of a composition comprising coated or uncoated digestive enzymes, wherein the diagnosis of the coronavirus infection is by standard reverse transcription polymerase chain reaction (RT-PCR) assay, an antibody test, an equivalent test, or other medically accepted techniques.

Provided herein is a method for diagnosing and treating a subject that is asymptomatic for COVID-19, comprising diagnosing the subject with a SARS-CoV-2 infection and then treating the subject by administering a therapeutically effective amount of a composition comprising coated or uncoated digestive enzymes, wherein the diagnosis of a SARS-CoV-2 infection/COVID-19 is by standard reverse transcription polymerase chain reaction (RT-PCR) assay, an antibody test, an equivalent test, or other medically accepted techniques.

Essential employees and first responders are at heightened risk of being infected with a coronavirus. Provided herein is a method for prophylaxis of subject who has been exposed to a coronavirus but is asymptomatic of a coronavirus and has not been diagnosed with a coronavirus Infection, comprising administering a therapeutically effective amount of a composition comprising coated or uncoated digestive enzymes to the subject.

Provided herein is a method for prophylaxis of subject who has been exposed to SARS-CoV-2 but is asymptomatic of COVID-19 and has not been diagnosed with SARS-CoV-2 infection, comprising administering a therapeutically effective amount of a composition comprising coated or uncoated digestive enzymes to the subject.

Also provided herein is a method for treating a subject with COVID-19, wherein the baseline severity of the COVID-19 is one of asymptomatic COVID-19, mild COVID-19, moderate COVID-19, severe COVID-19, or critical COVID-19.

Provided herein is a method for treating a subject having COVID-19, wherein the baseline severity of the infection is by diagnosis of one or more clinical signs suggestive of mild COVID-19 comprising one or more of fever, chills, cough, sore throat, fatigue, malaise, headache, muscle pain, body aches; however without dyspnea or shortness of breath and no signs of moderate, severe, or critical COVID-19, and positive testing by Standard Reverse Transcription Polymerase Chain Reaction (RT-PCR) assay, an antibody test, an equivalent test, or other medically accepted techniques.

Provided herein is a method for treating a subject with COVID-19, wherein the baseline severity of the infection is by diagnosis of one or more clinical signs suggestive of Moderate COVID-19 comprising one or more of: fever, chills, cough, sore throat, fatigue, malaise, headache, muscle pain, body aches, dyspnea, loss of taste, loss of smell, and gastrointestinal symptoms including, but not limited to nausea, vomiting, and diarrhea. shortness of breath with exertion, respiratory rate ≥20 breaths per minute, saturation of oxygen (SpO2) >93% on room air at sea level, heart rate ≥90 beats per minute, and no signs of severe or critical COVID-19 along with a positive testing by standard reverse transcription polymerase chain reaction (RT-PCR) assay, an antibody test, an equivalent test, or other medically accepted techniques.

Provided herein is a method for treating a subject with COVID-19, wherein the baseline severity of the infection is by diagnosis of one or more clinical signs suggestive of severe COVID-19 comprising one or more of: fever, chills, cough, sore throat, fatigue, malaise, headache, muscle pain, body aches, dyspnea, loss of taste, loss of smell, and gastrointestinal symptoms including, but not limited to, nausea, vomiting, and diarrhea shortness of breath at rest, respiratory distress, respiratory rate ≥30 per minute, heart rate ≥125 per minute, SpO2≤93% on room air at sea level or PaO2/FiO2<300, along with no signs of critical COVID-19 along with a positive testing by Standard Reverse Transcription Polymerase Chain Reaction (RT-PCR) assay, an antibody test, an equivalent test, or other medically accepted techniques.

Provided herein is a method for treating a subject with COVID-19, wherein the baseline severity of the infection is by diagnosis of one or more clinical signs suggestive of critical COVID-19 comprising one or more of: fever, chills, cough, sore throat, fatigue, malaise, headache, muscle pain, body aches, dyspnea, loss of taste, loss of smell, and gastrointestinal symptoms including, but not limited to nausea, vomiting, and diarrhea, clinical diagnosis of respiratory failure, respiratory failure requiring endotracheal intubation, mechanical ventilation, oxygen delivered by high flow nasal cannula, non-invasive positive pressure ventilation, extracorporeal membrane or other life-support machine, shock, shock defined by systolic blood pressure <90 mm Hg or diastolic blood pressure <60 mm Hg or requiring vasopressors), multi-organ dysfunction/failure, Standard Reverse Transcription Polymerase Chain Reaction (RT-PCR) assay, an antibody test, an equivalent test, or other medically accepted techniques.

In another embodiment, one or more symptoms of coronavirus infection (e.g., COVID-19), are selected from a group comprising fever, fever, chills, cough, sore throat, fatigue, malaise, headache, muscle pain, body aches, dyspnea, loss of taste, loss of smell, gastrointestinal symptoms (including, but not limited to nausea, vomiting, and diarrhea), shortness of breath with exertion, respiratory rate ≥20 breaths per minute, saturation of oxygen (SpO2) >93% on room air at sea level, heart rate >90 beats per minute, shortness of breath at rest, respiratory distress, respiratory rate ≥30 per minute, heart rate ≥125 per minute, SpO2≤93% on room air at sea level or PaO2/FiO2<300, clinical diagnosis of respiratory failure, respiratory failure requiring endotracheal intubation, mechanical ventilation, oxygen delivered by high flow nasal cannula, non-invasive positive pressure ventilation, extracorporeal membrane or other life-support machine, shock, shock defined by systolic blood pressure <90 mm Hg or diastolic blood pressure <60 mm Hg or requiring vasopressors), multi-organ dysfunction/failure, or a combination thereof.

Provided herein are compositions of digestive enzymes which are useful in the prevention (prophylaxis) or treatment of one or more symptoms of a coronavirus infection (e.g., symptoms of COVID-19). Digestive enzymes generally comprise one or more protease(s), one or more amylase(s), one or more lipase(s), or a combination thereof, and, optionally, other proteins (e.g., those secreted in a mammal) that affect the digestive process either directly or indirectly.

Treatment of coronaviruses (e.g., SARS-CoV-2) encompasses stasis of one or more symptoms (e.g., they do not worsen), as well as reduction (partial or complete) of one or more symptoms. In one embodiment, one or more symptoms caused by such coronaviruses are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100% following treatment compared to a subject that did not receive treatment, or compared to a subject that received a placebo. In another embodiment, one or more symptoms of infections caused by such coronaviruses are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold, or more, following treatment compared to a subject that did not receive treatment, or compared to a subject that received a placebo. In yet another embodiment, the duration of one or more symptoms may be reduced in severity and/or duration following administration of a composition described herein. That is, one or more symptoms may persist for less than 6 months, 5 months, 4 months, 3 months, 2 months, 1 month, 3 weeks, 2 weeks, or 1 week.

One or more digestive enzyme(s) may be utilized for targeted delivery against a specific coronavirus by targeting delivery to one or more coronavirus receptors including, but not limited to ACE2, mCEACAM, DDP4, APN, N-acetyl-9-O-acetylneuraminic acid, or a combination thereof. One or more digestive enzyme(s) may be coated for targeted delivery against a specific coronavirus by targeting delivery to one or more coronavirus receptors including, but not limited to ACE2, mCEACAM, DDP4, APN, N-acetyl-9-O-acetylneuraminic acid, or a combination thereof.

A targeted delivery system may be utilized to deliver one or more digestive enzyme(s) as a prophylaxis against infection from SARS-CoV-2 to the GI tract or mucosa including, but not limited to, one or more of the stomach, duodenum, small intestine, colon, and/or rectum. In another embodiment, a targeted delivery system may be utilized to nasally deliver one or more digestive enzyme(s) as treatment prophylaxis against infection with SARS-CoV-2 infection. In one embodiment, a targeted delivery system is utilized to deliver one or more digestive enzyme(s) as a prophylaxis against infection from SARS-CoV-2 to one or more ACE2 receptors.

In another embodiment, a targeted delivery system may be utilized to deliver one or more digestive enzyme(s) as treatment for COVID-19 or SARS-CoV-2 infection to the GI tract or mucosa including, but not limited to, one or more of the stomach, duodenum, small intestine, colon, and/or rectum. In another embodiment, a targeted delivery system may be utilized to nasally deliver one or more digestive enzyme(s) as treatment for SARS-CoV-2 infection. In another embodiment, a targeted delivery system is utilized to deliver one or more digestive enzyme(s) as treatment for COVID-19 or SARS-CoV-2 infection to one or more ACE2 receptors.

In another embodiment, a targeted delivery system is utilized to deliver one or more digestive enzyme(s) to the GI tract or mucosa as treatment for one or more of an asymptomatic, mild, moderate, severe, or critical COVID-19 including, but not limited to one or more of the stomach, duodenum, small intestine, colon, and/or rectum. In another embodiment, a targeted delivery system is utilized to nasally deliver one or more digestive enzyme(s) to a subject as treatment for one or more of an asymptomatic, mild, moderate, severe, or critical COVID-19. In another embodiment, a targeted delivery system is utilized to deliver one or more digestive enzyme(s) as treatment for one or more of an asymptomatic, mild, moderate, severe, or critical COVID-19 caused by a SARS-CoV-2 infection, to one or more ACE2 receptors.

In one embodiment, a targeted delivery system is utilized to deliver one or more digestive enzyme(s) as a prophylaxis against infection from Severe Acute Respiratory Syndrome Coronavirus (SARS-CoV) or Human Coronavirus NL63 (HCoV-NL63) infection to the GI tract including, but not limited to one or more of the stomach, duodenum, small intestine, colon, and/or rectum. In another embodiment, a targeted delivery system is utilized to deliver one or more digestive enzyme(s) as a prophylaxis against infection from SARS-CoV or HCoV-NL63, to one or more ACE2 receptors.

In another embodiment, a targeted delivery system is utilized to deliver one or more digestive enzyme(s) as treatment for SARS-CoV or HCoV-NL63 infection to the GI tract or mucosa including, but not limited to, one or more of the stomach, duodenum, small intestine, colon, and/or rectum. In another embodiment, a targeted delivery system is utilized to deliver one or more digestive enzyme(s) as treatment for SARS-CoV or HCoV-NL63 to one or more ACE2 receptors.

In another embodiment, a targeted delivery system is utilized to deliver one or more digestive enzyme(s) as treatment for one or more of an asymptomatic, mild, moderate, severe, or critical SARS-CoV or HCoV-NL63 infection to the GI tract including, but not limited to one or more of the stomach, duodenum, small intestine, colon, and/or rectum. In another embodiment, a targeted delivery system is utilized to deliver one or more digestive enzyme(s) as treatment for one or more of an asymptomatic, mild, moderate, severe, or critical SARS-CoV or HCoV-NL63 to one or more ACE2 receptors.

In one embodiment, a targeted delivery system is utilized to deliver one or more digestive enzyme(s) as a prophylaxis against infection from Human Coronavirus 229E (HCoV-229E), Transmissible Gastroenteritis Virus (TGEV), Porcine Epidemic Diarrhea Virus (PEDV), Feline Infectious Peritonitis Virus (FIPV), or Canine Coronavirus (CCoV) to the GI tract or mucosa including, but not limited to, one or more of the stomach, duodenum, small intestine, colon, and/or rectum. In another embodiment, a targeted delivery system is utilized to deliver one or more digestive enzyme(s) as a prophylaxis against infection from HCoV-229E, TGEV, PEDV, FIPV, or CCoV, to one or more APN receptors.

In another embodiment, a targeted delivery system is utilized to deliver one or more digestive enzyme(s) as treatment for HCoV-229E, TGEV, PEDV, FIPV, or CCoV infection to the GI tract including, but not limited to one or more of the stomach, duodenum, small intestine, colon, and/or rectum. In another embodiment, a targeted delivery system is utilized to deliver one or more digestive enzyme(s) as treatment for HCoV-229E, TGEV, PEDV, FIPV, or CCoV infection to one or more APN Receptors.

In another embodiment, a targeted delivery system is utilized to deliver one or more digestive enzyme(s) as treatment for one or more of an asymptomatic, mild, moderate, severe, or critical HCoV-229E, TGEV, PEDV, FIPV, or CCoV infection to the GI tract or mucosa including, but not limited to one or more of the stomach, duodenum, small intestine, colon, and/or rectum. In another embodiment, a targeted delivery system is utilized to deliver one or more digestive enzyme(s) as treatment for one or more of an asymptomatic, mild, moderate, severe, or critical HCoV-229E, TGEV, PEDV, FIPV, or CCoV infection to one or more APN Receptors.

In one embodiment, a targeted delivery system is utilized to deliver one or more digestive enzyme(s) as a prophylaxis against infection from Murine Hepatitis Virus (MHV) to the GI tract including, but not limited to one or more of the stomach, duodenum, small intestine, colon, and/or rectum. In another one embodiment, a targeted delivery system is utilized to deliver one or more digestive enzyme(s) as a prophylaxis against infection from the MHV to one or more mCEACAM also known as CEACAM1 Receptors.

In another embodiment, a targeted delivery system is utilized to deliver one or more digestive enzyme(s) as treatment for MHV infection to the GI tract or mucosa including, but not limited to one or more of the stomach, duodenum, small intestine, colon, and/or rectum. In another embodiment, a targeted delivery system is utilized to deliver one or more digestive enzyme(s) as treatment for MHV infection to one or more mCEACAM, also known as CEACAM1 Receptors.

In another embodiment, a targeted delivery system is utilized to deliver one or more digestive enzyme(s) as treatment for one or more of an asymptomatic, mild, moderate, severe, or critical MHV infection to the GI tract or mucosa including, but not limited to one or more of the stomach, duodenum, small intestine, colon, and/or rectum. In another embodiment, a targeted delivery system is utilized to deliver one or more digestive enzyme(s) as treatment for one or more of an asymptomatic, mild, moderate, severe, or critical MHV infection to one or more mCEACAM, also known as CEACAM1 Receptors.

In one embodiment, a targeted delivery system is utilized to deliver one or more digestive enzyme(s) as a prophylaxis against infection from MERS to the GI tract or mucosa including, but not limited to one or more of the stomach, duodenum, small intestine, colon, and/or rectum. In another embodiment, a targeted delivery system is utilized to deliver one or more digestive enzyme(s) as a prophylaxis against infection from the Middle East Respiratory Syndrome Coronavirus (MERS) infection to one or more DDP4 Receptors.

In another embodiment a targeted delivery system is utilized to deliver one or more digestive enzyme(s) as treatment for MERS infection to the GI tract or mucosa including, but not limited to one or more of the stomach, duodenum, small intestine, colon, and/or rectum. In another embodiment, a targeted delivery system is utilized to deliver one or more digestive enzyme(s) as treatment for MERS to one or more DDP4 Receptors.

In another embodiment, a targeted delivery system is utilized to deliver one or more digestive enzyme(s) as treatment for one or more of an asymptomatic, mild, moderate, severe, or critical MERS infection to the GI tract or mucosa including, but not limited to one or more of the stomach, duodenum, small intestine, colon, and/or rectum. In another embodiment, a targeted delivery system is utilized to deliver one or more digestive enzyme(s) as treatment for one or more of an asymptomatic, mild, moderate, severe, or critical MERS infection to one or more DDP4 Receptors.

In one embodiment, a targeted delivery system is utilized to deliver one or more digestive enzyme(s) as a prophylaxis against infection from Bovine Coronavirus (BCoV) to the GI tract or mucosa including, but not limited to, one or more of the stomach, duodenum, small intestine, colon, and/or rectum. In another embodiment, a targeted delivery system is utilized to deliver one or more digestive enzyme(s) as a prophylaxis against infection from the BCoV to one or more N-acetyl-9-O-acetylneuraminic Acid Receptors.

In another embodiment, a targeted delivery system is utilized to deliver one or more digestive enzyme(s) as treatment for BCoV infection to the GI tract including, but not limited to, to one or more of the stomach, duodenum, small intestine, colon, and/or rectum. In another embodiment a targeted delivery system is utilized to deliver one or more digestive enzyme(s) as treatment for BCoV infection, wherein the targeted delivery system comprises one or more digestive enzyme(s) that cleave one or more N-acetyl-9-O-acetylneuraminic acid receptors.

In another embodiment, a targeted delivery system is utilized to deliver one or more digestive enzyme(s) as treatment for one or more of an asymptomatic, mild, moderate, severe, or critical BCoV infection to the GI tract including, but not limited to one or more of to one or more of the stomach, duodenum, small intestine, colon, and/or rectum. In another embodiment a targeted delivery system is utilized to deliver one or more digestive enzyme(s) as treatment for one or more of an asymptomatic, mild, moderate, severe, or critical BCoV infection to one or more of to one or more N-acetyl-9-O-acetylneuraminic acid receptors.

The diagnostic criteria described above may be used to assess whether administration of a composition described herein reduces the severity and/or duration of one or more symptoms of a coronavirus infection.

In another embodiment, the pharmaceutical composition comprising digestive enzymes in a pH buffered solution of saline solution is administered 1, 2, 3, 4, 5 6, 7, 8, 9, or 10 times or more a day to act as a prophylaxis against a coronavirus infection or a treatment of coronaviruses.

In one embodiment the pharmaceutical composition comprising digestive enzymes in a pH buffered saline solution is administered as a prophylaxis against infection from SARS-CoV-2.

In another embodiment, the pharmaceutical composition comprising digestive enzymes in a solution pH buffered saline solution is administered as treatment for COVID-19.

In another embodiment, the digestive enzymes in the nasal spray, nasal drops, or nasal wash comprise porcine digestive enzymes. In yet another embodiment, the porcine enzymes comprise one or more protease(s), one or more amylase(s), one or more lipase(s), or a combination thereof. In yet another embodiment, the porcine enzymes comprise one or more protease(s), one or more amylase(s), and one or more lipase(s).

In another embodiment, the digestive enzymes in the pharmaceutical composition comprises porcine digestive enzymes within a solution of approximately 1%, 2%, 3%, 4%, 5%, 6%, 7%, 8%, 9%, 10%, 11%, 12%, 13%, 14%, or 15% or more by weight. In yet another embodiment, the pharmaceutical composition comprises porcine digestive enzymes having an activity of approximately 20,000 or more U.S.P. Units protease, 15,000 or more U.S.P. Units amylase, 4,000 or more U.S.P. Units lipase in a 10 ml pH buffered saline solution

Optionally, a solution of collagen or similarly enzymatically digestible target protein in a pH-buffered saline solution is administered with approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes or more for a duration of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes or more after administering the pharmaceutical composition comprising the digestive enzymes to neutralize the effect of the digestive enzymes.

### Digestive Enzymes

Compositions provided herein may include not only one or more digestive enzyme(s), but optionally, also one or more pharmaceutically acceptable carriers, excipients, buffers, fillers, binders, stabilizers, surfactants, diluents, extracts, lubricants, fillers, flavorings, preservatives, colorants, diluents, anti-microbials, disintegrants, and coating agents, such as for example, vegetable oil, crystalline oils, taste maskers, sweeteners, etc. In one embodiment, digestive enzymes are provided as a pharmaceutical composition.

In one embodiment, a composition may contain one or more protease(s), one or more amylase(s), one or more lipase(s), or a combination thereof. A composition may contain one or more protease(s). A composition may contain one or more amylase(s). A pharmaceutical composition may contain one or more lipase(s). A composition may contain one or more protease(s) and one or more amylase(s). A composition may contain one or more protease(s) and one or more lipase(s). A composition may contain one or more amylase(s) and one or more lipase(s). A composition may contain one or more protease(s), one or more amylase(s), and one or more lipase(s).

The one or more protease(s) can comprise, for example, papain, bromelain, chymotrypsin, trypsin, Carboxypeptidase B, serine proteases (e.g., Kallikrein), or a combination thereof. In one instance, the one or more protease(s) comprise chymotrypsin. In one instance, the one or more protease(s) comprise trypsin. In one instance, the one or more protease(s) comprise chymotrypsin and trypsin. Other enzymes that may be utilized in the pharmaceutical compositions described herein include, but are not limited to, an elastase, a sucrase, a maltase, a cellulase, a hydrolase, a colipase, a Phospholipase A2, a Cholesterol Esterase, or a combination thereof. Digestive enzymes may also be provided, in some instances, as pancreatin, pancrealipase, or derived therefrom. Pancreatin may be, for example, a crystalline pancreatin.

The one or more digestive enzyme(s) can be, independently, derived from an animal source, a microbial source, a plant source, are recombinantly-prepared, or are synthetically-prepared. In some embodiments, the animal source is a pig, e.g., a pig pancreas, or avian, e.g., “bird” proventriculus or small intestine. The digestive enzymes can be any combination of digestive enzymes of a type produced by the pancreas, including, but not limited to digestive enzymes from a pancreatic source or other source. The scope of the disclosure is not limited to pancreatic enzymes of porcine origin but can be of animal origin, microbial origin, plant origin, as well as those that are recombinantly-derived or synthetically-derived. In one embodiment, the digestive enzyme is derived from mammalian sources such as porcine-derived digestive enzymes. In another embodiment, the digestive enzyme includes one or more enzymes, and is plant-derived, synthetically-derived, or recombinantly-produced in microbial cells, yeast cells, or mammalian cells, or includes a mixture of enzymes from one or more source(s). For example, digestive enzymes may include one or more enzyme(s) from one or more source(s) mixed together. This includes, for example, the addition of single digestive enzymes to digestive enzymes derived from pancreatic sources in order to provide appropriate levels of specific enzymes that provide more effective treatment for a selected disease or condition. One exemplary source of pancreatin digestive enzymes can be obtained, for example, from Scientific Protein Laboratories. In one non-limiting embodiment provided herein utilizes one or more of the following enzymes: trypsin, chymotrypsin, elastase, kallikrein, carboxypeptidase b, alpha-amylase, lipase, colipase, phospholipase A2, cholesterol, esterase, or a combination thereof. In one embodiment the digestive enzymes are pancreatic digestive enzymes. In one embodiment, the animal enzyme is derived from a mammal. In one embodiment the mammal is a pig. In one embodiment, digestive enzymes are derived from a mammalian pancreas. In one embodiment the pancreas is a pig pancreas or avian, e.g., “bird” proventriculus or small intestine. In yet another embodiment, digestive enzymes as well as other proteins (e.g., those secreted in a mammal) that affect the digestive process either directly or indirectly may be included in a composition.

In any of such targeted delivery systems described above, the one or more digestive enzyme(s) can comprise one or more protease(s), one or more lipase(s), one or more amylase(s), or a combination thereof. For example, the one or more digestive enzyme(s) can comprise one or more protease(s); the digestive enzymes can comprise one or more lipase(s); the digestive enzymes can comprise one or more amylase(s); the digestive enzymes can comprise one or more protease(s) and one or more lipase(s); the digestive enzymes can comprise one or more protease(s) and one or more amylase(s); the digestive enzymes can comprise one or more lipase(s) and one or more amylase(s); or the digestive enzymes can comprise one or more protease(s), one or more lipase(s), and one or more amylase(s). In one non-limiting embodiment, the digestive enzymes in the targeted delivery system are provided as pancreatin.

In another embodiment, the digestive enzymes in the targeted delivery system are uncoated and, optionally, may be formulated with one or more carriers, excipients, buffers, fillers, binders, stabilizers, surfactants, diluents, extracts, lubricants, fillers, flavorings, preservatives, colorants, diluents, and coating agents, such as vegetable oil, crystalline oils, taste maskers, sweeteners, etc.

In one embodiment, a total amount of protease in a composition ranges from about 5,000 to about 1,500,000 U.S.P. Units/dose. In another embodiment, a total amount of amylase in a composition ranges from about 1,000 to about 15,000,000 U.S.P. Units/dose. In another embodiment, a total amount of lipase in a composition ranges from about 1,500 to about 282,000 U.S.P. Units/dose.

Provided herein is a composition comprising digestive enzymes for use in the methods described herein, wherein the digestive enzymes comprise about 23,000 U.S.P. Units/dose of lipase, about 144,000 U.S.P. Units/dose of amylase and about 140,000 U.S.P. Units/dose of protease. Provided herein is a composition comprising digestive enzymes for use in the methods described herein, wherein the digestive enzymes comprise about 23,040 U.S.P. Units/dose of lipase, about 144,000 U.S.P. Units/dose of amylase and about 140,400 U.S.P. Units/dose of protease. Provided herein is a composition comprising digestive enzymes for use in the methods described herein, wherein the digestive enzymes comprise about 16,800 U.S.P. Units/dose of lipase, about 70,000 U.S.P. Units/dose of protease, and about 70,000 U.S.P. Units/dose of amylase. Provided herein is a composition comprising digestive enzymes for use in the methods described herein, wherein the digestive enzymes comprise about 16,800 U.S.P. Units/dose of lipase, about 110,000 U.S.P. Units/dose of protease, and about 70,000 U.S.P. Units/dose of amylase. In one embodiment, a composition comprises about 8,400 U.S.P. Units/dose lipase, 35,000 U.S.P. Units/dose protease, and about 35,000 U.S.P. Units/dose amylase. In one embodiment, a composition comprises about 8,400 U.S.P. Units/dose lipase, 35,000 U.S.P. Units/dose protease, and about 35,000 U.S.P. Units/dose amylase. In one embodiment, a composition comprises about 16,800 U.S.P. Units/dose lipase, about 70,000 U.S.P. Units/dose protease, and about 70,000 U.S.P. Units/dose amylase. In one embodiment, a composition comprises about 33,600 U.S.P. Units/dose lipase, about 140,000 U.S.P. Units/dose protease, and about 140,000 U.S.P. Units/dose amylase. In one embodiment, a composition comprises about 50,400 U.S.P. Units/dose lipase, about 210,000 U.S.P. Units/dose protease, and about 210,000 U.S.P. Units/dose amylase. In one embodiment, a composition comprises about 67,200 U.S.P. Units/dose lipase, about 280,000 U.S.P. Units/dose protease, and about 280,000 U.S.P. Units/dose amylase. In one embodiment, a composition comprises about 84,000 U.S.P. Units/dose lipase, about 350,000 U.S.P. Units/dose protease, and about 350,000 U.S.P. Units/dose amylase. In one embodiment, a composition comprises about 100,800 U.S.P. Units/dose lipase, about 420,000 U.S.P. Units/dose protease, and about 420,000 U.S.P. Units/dose amylase. In one embodiment, a composition comprises about 117,600 U.S.P. Units/dose lipase, about 490,000 U.S.P. Units/dose protease, and about 490,000 U.S.P. Units/dose amylase. In one embodiment, a composition comprises about 134,400 U.S.P. Units/dose lipase, about 560,000 U.S.P. Units/dose protease, and about 560,000 U.S.P. Units/dose amylase. In one embodiment, a composition comprises about 151,200 U.S.P. Units/dose lipase, about 630,000 U.S.P. Units/dose protease, and about 630,000 U.S.P. Units/dose amylase. In one embodiment, a composition comprises about 168,000 U.S.P. Units/dose lipase, about 700,000 U.S.P. Units/dose protease, and about 700,000 U.S.P. Units/dose amylase. In one embodiment, a composition comprises about 184,800 U.S.P. Units/dose lipase, about 770,000 U.S.P. Units/dose protease, and about 770,000 U.S.P. Units/dose amylase. In one embodiment, a composition comprises about 201,600 U.S.P. Units/dose lipase, about 840,000 U.S.P. Units/dose protease, and about 840,000 U.S.P. Units/dose amylase. In one embodiment, a composition comprises about 218,400 U.S.P. Units/dose lipase, about 910,000 U.S.P. Units/dose protease, and about 910,000 U.S.P. Units/dose amylase. In one embodiment, a composition comprises about 235,200 U.S.P. Units/dose lipase, about 980,000 U.S.P. Units/dose protease, and about 980,000 U.S.P. Units/dose amylase. In one embodiment, a composition comprises about 252,000 U.S.P. Units/dose lipase, about 1,050,000 U.S.P. Units/dose protease, and about 1,050,000 U.S.P. Units/dose amylase.

Any of the compositions may be optionally supplemented with additional protease. In one embodiment, a composition may be supplemented with about 20,000 U.S.P. Units/dose; about 40,000 U.S.P. Units/dose; about 60,000 U.S.P. Units/dose; about 80,000 U.S.P. Units/dose; about 100,000 U.S.P. Units/dose; about 120,000 U.S.P. Units/dose; about 140,000 U.S.P. Units/dose; or about 160,000 U.S.P. Units/dose protease.

In some embodiments, the digestive enzyme composition comprises a protease, a lipase, and an amylase where the activities are: protease in an amount of from about 10,000 to about 1,500,000 United States Pharmacopeia (U.S.P.) units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between, and where the ratio of protease to lipase is such that the amount of lipase is never more than 0.188 times the amount of protease and where the ratio of protease activity to amylase activity is between 1:0.1 and 1:10.

In some embodiments, the digestive enzyme composition comprises a protease and a lipase, where the activities are: protease in an amount of from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between, and where the ratio of protease to lipase is such that the amount of lipase is never more than 0.188 times the amount of protease.

In some embodiments, the digestive enzyme composition comprises a protease and an amylase where the activities are: protease in an amount of from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between, and where the ratio of protease activity to amylase activity is between 1:0.1 and 1:10.

In some embodiments, the digestive enzyme composition comprises a protease where the activity is: protease in an amount of from about 10,000 to about 1,500,000 U.S.P. Units/dose including about 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between.

In some embodiments, the digestive enzyme composition comprises a protease, a lipase, and an amylase where the activities are: protease in an amount of from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between; lipase from about 1,880 to about 282,000 U.S.P. Units/dose including about any of 1,880, 10,000, 50,000, 100,000, 150,000, 200,000, 250,000, 282,000, per dose, along with all values in-between; amylase in amount of from about 1,000 to about 15,000,000 U.S.P. Units/dose, including about any of 1,000, 10,000, 100,000, 500,000, 1,000,000, 2,000,000, 3,000,000, 4,000,000, 5,000,000, 6,000,000, 7,000,000, 8,000,000, 9,000,000, 10,000,000, 11,000,000, 12,000,000, 13,000,000, 14,000,000, and 15,000,000 U.S.P. Units, per dose, along with all values in-between.

In some embodiments, the digestive enzyme composition is comprised of protease and lipase, where the activities are: protease from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between; and lipase from about 1,880 to about 282,000 U.S.P. Units/dose including about any of 1,880, 10,000, 50,000, 100,000, 150,000, 200,000, 250,000, and 282,000, per dose, along with all values in-between.

In some embodiments, the digestive enzyme composition comprises a protease and an amylase where the activities are: protease in an amount of from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between; amylase in an amount of from about 1,000 to about 15,000,000 U.S.P. Units/dose, including about any of 1,000, 10,000, 100,000, 500,000, 1,000,000, 2,000,000, 3,000,000, 4,000,000, 5,000,000, 6,000,000, 7,000,000, 8,000,000, 9,000,000, 10,000,000, 11,000,000, 12,000,000, 13,000,000, 14,000,000, and 15,000,000 U.S.P. Units/dose, per dose, along with all values in-between.

In some embodiments, the digestive enzyme composition comprises a protease, where the activity is: protease in an amount of from about 10,000 to about 1,500,000 U.S.P, units including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between.

In some embodiments, the digestive enzyme composition comprises at least one amylase, a mixture of proteases comprising chymotrypsin and trypsin, and at least one lipase. The pharmaceutical composition can, optionally, further include papain (e.g., from papaya) or bromelain. In some embodiments, the coated pharmaceutical composition comprises per dose: amylase in amount of from about 1,000 to about 15,000,000 U.S.P. Units/dose, including about any of 1,000, 10,000, 100,000, 500,000, 1,000,000, 2,000,000, 3,000,000, 4,000,000, 5,000,000, 6,000,000, 7,000,000, 8,000,000, 9,000,000, 10,000,000, 11,000,000, 12,000,000, 13,000,000, 14,000,000, or 15,000,000 U.S.P. Units/dose, along with all values in-between; protease in an amount of from about 10,000 to about 1,500,000 U.S.P. Units/dose including about any of 10,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,050,000, 1,100,000, 1,150,000, 1,200,00, 1,250,000, 1,300,000, 1,350,000, 1,400,000, 1,450,000, and 1,500,000 per dose, along with all values in-between, lipase in amount of from about 1,880 to about 282,000 U.S.P. Units/dose including about any of 1,880, 10,000, 50,000, 100,000, 150,000, 200,000, 250,000, and 282,000 per dose, along with all values in-between.

A pharmaceutical composition may contain an amount of protease of from about 1,000 to about 15,000,000 U.S.P. Units/dose. For example, a pharmaceutical composition may contain an amount of protease from about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000 U.S.P. Units/dose, or any integer in-between.

A pharmaceutical composition may contain an amount of amylase of from about 1,000 to about 15,000,000; from about 5,000 to about 1,000,000; from about 15,000 to about 750,000; from about 50,000 to about 500,000; from about 75,000 to about 250,000; from about 95,000 to about 200,000; or from about 100,000 to about 150,000 U.S.P. Units/dose. For example, a pharmaceutical composition may contain an amount of amylase including, but not limited to, about 1,000; about 3,000; about 5,000; about 7,500; about 10,000; about 15,000; about 20,000; about 25,000; about 30,000; about 35,000; about 40,000; about 50,000; about 65,000; about 70,000; about 75,000; about 100,000; about 140,000; about 144,000; about 210,000; about 280,000; about 350,000; about 420,000; about 490,000; about 500,000; about 560,000; about 630,000; about 700,000; about 770,000; about 840,000; about 910,000; about 980,000; about 1,000,000; about 1,050,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; or about 15,000,000 U.S.P. Units/dose, or any integer in-between.

A pharmaceutical composition may contain an amount of lipase of from about 1,500 to about 282,000; from about 5,000 to about 200,000; from about 5,000 to about 150,000; from about 75,000 to about 100,000; from about 10,000 to about 75,000; from about 15,000 to about 50,000; or from about 20,000 to about 40,000 U.S.P. Units/dose. For example, a pharmaceutical composition may contain an amount of lipase including, but not limited to, about 1,500; about 1,880; about 2,000; about 3,000; about 5,000; about 7,500; about 8,400; about 10,000; about 15,000; about 16,800; about 20,000; about 23,000; about 23,040; about 25,000; about 30,000; about 33,600; about 40,000; about 50,000; about 50,400; about 65,000; about 67,200; about 75,000; about 84,000; about 100,000; about 100,800; about 117,600; about 125,000; about 134,400; about 150,000; about 151,200; about 168,000; about 184,800; about 200,000; about 201,600; about 218,400; about 235,200; about 250,000; about 252,000; or about 282,000 U.S.P. Units/dose, or any integer in-between.

In one instance, a pharmaceutical composition comprises a protease, a lipase, and an amylase where the activity of the protease is from about 5,000 to about 1,500,000 U.S.P. Units/dose, or from about 10,000 to about 1,500,000 U.S.P. Units/dose including, but not limited to, about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000 U.S.P. Units/dose, or any integer in-between, and where the ratio of protease to lipase is such that the amount of lipase is never more than 0.188 times the amount of protease; and further wherein the ratio of protease activity to amylase activity is between 1:0.1 and 1:10.

In some embodiments, the digestive enzyme composition comprises at least one protease and at least one lipase, wherein the ratio of total protease to total lipase in U.S.P. Units ranges from about 5.371:1 to about 20:1 including 5.371:1, 6:1, 7:1, 8:1, 9:1, 10:1, 11:1, 12;1, 13;1, 14:1, 15:1, 16;1, 17:1, 18:1, 19:1 and 20:1, along with all values in-between. In some embodiments, the ratio of protease to lipase in U.S.P. Units ranges from about 5.371:1 to about 10:1 including 5.371:1, 6:1, 7:1, 8:1, 9:1, and 10:1, along with all values in-between.

In yet another embodiment, the digestive enzyme composition comprises at least one protease and at least one lipase, wherein the ratio of total protease to total lipase in U.S.P. Units ranges from about 5.371:1 to about 20:1 including about 1:1, 2:1, 3:1, 4:1, 5:1 (e.g., 5.371:1), 6:1, 7:1, 8:1, 9:1, 10:1, 11:1, 12:1, 13:1, 14:1, 15:1, 16:1, 17:1, 18:1, 19:1, and 20:1, along with all values in-between. In another embodiment, the digestive enzyme composition comprises at least one protease and at least one lipase, wherein the ratio of total protease to total lipase in U.S.P. Units ranges from about 1:1 to about 20:1. In yet another embodiment, the ratio of protease to lipase in U.S.P. Units ranges from about 4:1 to about 10:1. In one embodiment, the ratio of proteases to lipases in U.S.P. Units ranges from about 5:1 (e.g., 5.371:1) to about 10:1 including 5:1 (e.g., 5.371:1), 6:1, 7:1, 8:1, 9:1, and 10:1, or any integer in-between.

In one embodiment, the digestive enzyme composition comprises at least one protease and at least one amylase, wherein the ratio of total protease to total amylase in in U.S.P. Units ranges from about 1:0.1 to about 1:10 including 1:0.1, 1:0.25, 1:0.5, 1:0.75, 1:1, 1:1.25, 1:1.5, 1:1.75:1:2, 1:1.25, 1:1.5, 1:1.75, 1:1.2, 1:1.25, 1:1.5, 1:1.75, 1:1.2, 1:1.25, 1:1.5, 1:1.75, 1:1.2:1:1.5, 1:2, 1:3, 1:4, 1:5, 1:6, 1:7, 1:8, 1.9 and 1:10, along any integer in-between.

In another embodiment, the digestive enzyme composition comprises at least one protease and at least one amylase, wherein the ratio of total protease to total amylase in U.S.P. Units ranges from about 1:6 to about 1:0.14 including any integer in-between. In another embodiment, the ratio of protease to amylase in U.S.P. Units ranges from about 1:7 to about 1:0.125 including any integer in-between.

In another embodiment, the digestive enzyme composition comprises a protease, a lipase, and an amylase where the activities are: protease in an amount of from about 5,000 to about 1,500,000 U.S.P. Units/dose, or from about 10,000 to about 1,500,000 U.S.P. Units/dose, including about 5,000, about 7,500, about 10,000, about 15,000, about 20,000, about 25,000, about 30,000, about 35,000, about 40,000, about 50,000, about 55,000, about 65,000, about 70,000, about 75,000, about 90,000, about 95,000, about 100,000, about 110,000, about 115,000, about 130,000, about 140,000, about 140,400, about 150,000, about 155,000, about 160,000, about 170,000, about 175,000, about 180,000, about 190,000, about 195,000, about 200,000, about 210,000, about 220,000, about 230,000, about 240,000, about 250,000, about 280,000, about 290,000, about 300,000, about 310,000, about 320,000, about 330,000, about 340,000, about 350,000, about 360,000, about 370,000, about 380,000, about 390,000, about 400,000, about 410,000, about 420,000, about 430,000, about 440,000, about 450,000, about 465,000, about 470,000, about 480,000, about 490,000, about 500,000, about 510,000, about 520,000, about 530,000, about 540,000, about 550,000, about 560,000, about 570,000, about 580,000, about 590,000, about 600,000, about 610,000, about 620,000, about 630,000, about 640,000, about 650,000, about 660,000, about 670,000, about 680,000, about 690,000, about 700,000, about 710,000, about 720,000, about 730,000, about 740,000, about 750,000, about 760,000, about 770,000, about 780,000, about 790,000, about 800,000, about 810,000, about 820,000, about 830,000, about 840,000, about 850,000, about 860,000, about 870,000, about 880,000, about 890,000, about 900,000, about 910,000, about 920,000, about 930,000, about 940,000, about 950,000, about 960,000, about 970,000, about 980,000, about 990,000, about 1,000,000, about 1,010,000, about 1,020,000, about 1,020,000, about 1,030,000, about 1,040,000, about 1,050,000, about 1,060,000, about 1,070,000, about 1,080,000, about 1,090,000, about 1,100,000, about 1,100,000, about 1,120,000, about 1,130,000, about 1,140,000, about 1,150,000, about 1,170,000, about 1,190,000, about 1,200,000, about 1,210,000, about 1,250,000, about 1,300,000, about 1,350,000, about 1,400,000, about 1,450,000, or about 1,500,000 U.S.P. Units/dose, or any integer in-between, and where the ratio of protease to lipase is such that the amount of lipase is never more than 0.188 times the amount of protease and where the ratio of protease activity to amylase activity is from about 1:0.1 and about 1:10.

In yet another embodiment, the digestive enzyme composition comprises at least one protease wherein the activity of protease is from about 5,000, about 7,500, about 10,000, about 15,000, about 20,000, about 25,000, about 30,000, about 35,000, about 40,000, about 50,000, about 55,000, about 65,000, about 70,000, about 75,000, about 90,000, about 95,000, about 100,000, about 110,000, about 115,000, about 130,000, about 140,000, about 140,400, about 150,000, about 155,000, about 160,000, about 170,000, about 175,000, about 180,000, about 190,000, about 195,000, about 200,000, about 210,000, about 220,000, about 230,000, about 240,000, about 250,000, about 280,000, about 290,000, about 300,000, about 310,000, about 320,000, about 330,000, about 340,000, about 350,000, about 360,000, about 370,000, about 380,000, about 390,000, about 400,000, about 410,000, about 420,000, about 430,000, about 440,000, about 450,000, about 465,000, about 470,000, about 480,000, about 490,000, about 500,000, about 510,000, about 520,000, about 530,000, about 540,000, about 550,000, about 560,000, about 570,000, about 580,000, about 590,000, about 600,000, about 610,000, about 620,000, about 630,000, about 640,000, about 650,000, about 660,000, about 670,000, about 680,000, about 690,000, about 700,000, about 710,000, about 720,000, about 730,000, about 740,000, about 750,000, about 760,000, about 770,000, about 780,000, about 790,000, about 800,000, about 810,000, about 820,000, about 830,000, about 840,000, about 850,000, about 860,000, about 870,000, about 880,000, about 890,000, about 900,000, about 910,000, about 920,000, about 930,000, about 940,000, about 950,000, about 960,000, about 970,000, about 980,000, about 990,000, about 1,000,000, about 1,010,000, about 1,020,000, about 1,020,000, about 1,030,000, about 1,040,000, about 1,050,000, about 1,060,000, about 1,070,000, about 1,080,000, about 1,090,000, about 1,100,000, about 1,100,000, about 1,120,000, about 1,130,000, about 1,140,000, about 1,150,000, about 1,170,000, about 1,190,000, about 1,200,000, about 1,210,000, about 1,250,000, about 1,300,000, about 1,350,000, about 1,400,000, about 1,450,000, or about 1,500,000 U.S.P. Units/dose or any integer in-between.

In yet another embodiment, the digestive enzyme composition comprises one or more coated or uncoated protease(s), wherein the activity of protease is from about 10,000 to about 1,500,000, from about 25,000 to about 1,000,000, from about 50,000 to about 750,000, from about 75,000 to about 500,000, from about 85,000 to about 250,000, from about 95,000 to about 200,000, or from about 110,000 to about 150,000 U.S.P. Units/dose. Compositions may contain an amount of protease including, but not limited to, about 5,000, about 7,500, about 10,000, about 15,000, about 20,000, about 25,000, about 30,000, about 35,000, about 40,000, about 50,000, about 55,000, about 65,000, about 70,000, about 75,000, about 90,000, about 95,000, about 100,000, about 110,000, about 115,000, about 130,000, about 140,000, about 140,400, about 150,000, about 155,000, about 160,000, about 170,000, about 175,000, about 180,000, about 190,000, about 195,000, about 200,000, about 210,000, about 220,000, about 230,000, about 240,000, about 250,000, about 280,000, about 290,000, about 300,000, about 310,000, about 320,000, about 330,000, about 340,000, about 350,000, about 360,000, about 370,000, about 380,000, about 390,000, about 400,000, about 410,000, about 420,000, about 430,000, about 440,000, about 450,000, about 465,000, about 470,000, about 480,000, about 490,000, about 500,000, about 510,000, about 520,000, about 530,000, about 540,000, about 550,000, about 560,000, about 570,000, about 580,000, about 590,000, about 600,000, about 610,000, about 620,000, about 630,000, about 640,000, about 650,000, about 660,000, about 670,000, about 680,000, about 690,000, about 700,000, about 710,000, about 720,000, about 730,000, about 740,000, about 750,000, about 760,000, about 770,000, about 780,000, about 790,000, about 800,000, about 810,000, about 820,000, about 830,000, about 840,000, about 850,000, about 860,000, about 870,000, about 880,000, about 890,000, about 900,000, about 910,000, about 920,000, about 930,000, about 940,000, about 950,000, about 960,000, about 970,000, about 980,000, about 990,000, about 1,000,000, about 1,010,000, about 1,020,000, about 1,020,000, about 1,030,000, about 1,040,000, about 1,050,000, about 1,060,000, about 1,070,000, about 1,080,000, about 1,090,000, about 1,100,000, about 1,100,000, about 1,120,000, about 1,130,000, about 1,140,000, about 1,150,000, about 1,170,000, about 1,190,000, about 1,200,000, about 1,210,000, about 1,250,000, about 1,300,000, about 1,350,000, about 1,400,000, about 1,450,000, or about 1,500,000, U.S.P. Units/dose, or any integer in-between. An added benefit is that this formulation will be useful in very young infants who are not able to tolerate lipase activity.

A dose of a pharmaceutical composition can comprise from about 100 mg to about 1500 mg, from 500 mg to about 1200 mg, from about 800 mg to about 1000 mg, or from about 850 mg to about 950 mg of digestive enzymes by weight. In one instance, a dose of a pharmaceutical composition can comprise from about 100 mg to about 1500 mg of digestive enzymes by weight. In another instance, a dose of a pharmaceutical composition can comprise from 500 mg to about 1200 mg of digestive enzymes by weight. In another instance, a dose of a pharmaceutical composition can comprise from about 800 mg to about 1000 mg of digestive enzymes by weight. In another instance, a dose of a pharmaceutical composition can comprise from about 850 mg to about 950 mg of digestive enzymes by weight.

A dose of a pharmaceutical composition can comprise about 100, about 150, about 200, about 250, about 300, about 350, about 400, about 450, about 500, about 550, about 600, 650, about 700, about 750, about 800, about 850, about 900, about 950, about 1000, about 1050, about 1100, about 1150, about 1200, about 1250, about 1300, about 1350, about 1400, about 1450, or about 1500 mg of digestive enzymes by weight. In one instance, a dose of a pharmaceutical composition can comprise about 800, about 810, about 820, about 830, about 840, about 850, about 860, about 870, about 880, about 890, about 900, about 910, about 920, about 930, about 940, about 950, about 960, about 970, about 980, about 990, or about 1000 mg of digestive enzymes by weight. In one non-limiting instance, a dose of a pharmaceutical composition comprises about 850 mg of digestive enzymes by weight. In another non-limiting instance, a dose of a pharmaceutical composition comprises about 860 mg of digestive enzymes by weight. In another non-limiting instance, a dose of a pharmaceutical composition comprises about 870 mg of digestive enzymes by weight. In another non-limiting instance, a dose of a pharmaceutical composition comprises about 880 mg of digestive enzymes by weight. In another non-limiting instance, a dose of a pharmaceutical composition comprises about 890 mg of digestive enzymes by weight. In another non-limiting instance, a dose of a pharmaceutical composition comprises about 900 mg of digestive enzymes by weight. In another non-limiting instance, a dose of a pharmaceutical composition comprises about 910 mg of digestive enzymes by weight. In another non-limiting instance, a dose of a pharmaceutical composition comprises about 920 mg of digestive enzymes by weight. In another non-limiting instance, a dose of a pharmaceutical composition comprises about 930 mg of digestive enzymes by weight. In another non-limiting instance, a dose of a pharmaceutical composition comprises about 940 mg of digestive enzymes by weight. In another non-limiting instance, a dose of a pharmaceutical composition comprises about 950 mg of digestive enzymes by weight.

A dose of a pharmaceutical composition can comprise a protease activity of not less than (NTL) about 100, about 105, about 110, about 115, about 120, about 125, about 130, about 135, about 140, about 145, about 150, about 155, about 156, about 160, about 165, about 170, about 175, about 180, about 185, about 190, about 195, or about 200 U.S.P. units/mg. In one non-limiting instance, a dose of a pharmaceutical composition can comprise a protease activity of not less than about 100 U.S.P. units/mg. In another non-limiting instance, a dose of a pharmaceutical composition can comprise a protease activity of not less than about 105 U.S.P. units/mg. In another non-limiting instance, a dose of a pharmaceutical composition can comprise a protease activity of not less than about 110 U.S.P. units/mg. In another non-limiting instance, a dose of a pharmaceutical composition can comprise a protease activity of not less than about 115 U.S.P. units/mg. In another non-limiting instance, a dose of a pharmaceutical composition can comprise a protease activity of not less than about 120 U.S.P. units/mg. In another non-limiting instance, a dose of a pharmaceutical composition can comprise a protease activity of not less than about 125 U.S.P. units/mg. In another non-limiting instance, a dose of a pharmaceutical composition can comprise a protease activity of not less than about 130 U.S.P. units/mg. In another non-limiting instance, a dose of a pharmaceutical composition can comprise a protease activity of not less than about 135 U.S.P. units/mg. In another non-limiting instance, a dose of a pharmaceutical composition can comprise a protease activity of not less than about 140 U.S.P. units/mg. In another non-limiting instance, a dose of a pharmaceutical composition can comprise a protease activity of not less than about 145 U.S.P. units/mg. In another non-limiting instance, a dose of a pharmaceutical composition can comprise a protease activity of not less than about 150 U.S.P. units/mg. In another non-limiting instance, a dose of a pharmaceutical composition can comprise a protease activity of not less than about 155 U.S.P. units/mg. In another non-limiting instance, a dose of a pharmaceutical composition can comprise a protease activity of not less than about 156 U.S.P. units/mg. In another non-limiting instance, a dose of a pharmaceutical composition can comprise a protease activity of not less than about 160 U.S.P. units/mg. In another non-limiting instance, a dose of a pharmaceutical composition can comprise a protease activity of not less than about 165 U.S.P. units/mg. In another non-limiting instance, a dose of a pharmaceutical composition can comprise a protease activity of not less than about 170 U.S.P. units/mg. In another non-limiting instance, a dose of a pharmaceutical composition can comprise a protease activity of not less than about 175 U.S.P. units/mg. In another non-limiting instance, a dose of a pharmaceutical composition can comprise a protease activity of not less than about 180 U.S.P. units/mg. In another non-limiting instance, a dose of a pharmaceutical composition can comprise a protease activity of not less than about 185 U.S.P. units/mg. In another non-limiting instance, a dose of a pharmaceutical composition can comprise a protease activity of not less than about 190 U.S.P. units/mg. In another non-limiting instance, a dose of a pharmaceutical composition can comprise a protease activity of not less than about 195 U.S.P. units/mg. In another non-limiting instance, a dose of a pharmaceutical composition can comprise a protease activity of not less than about 200 U.S.P. units/mg.

Exemplary compositions comprising an effective amount of digestive enzymes may be administered to a subject via any conventional route including, but not limited to, oral, parenteral, intramuscular, intravenous, transmucosal, transdermal, nasal, rectal (e.g., suppository), percutaneous endoscopic gastrostomy (PEG), esophagogastroduodenoscopy (EGD), gastrostomy (G-tube) insertion, or any other appropriate delivery method. Further, the oral administration can be in the form of solutions, slurries, suspensions, sprays, washes, rinses, pellets, capsules, caplets, beadlets, sprinkles, tablets, softgels, sachets, pouches, or other. Nasal administration can be, for example, a nasal spray, a nasal drop, or a nasal rinse.

Compositions (preparations) comprising digestive enzymes may be manufactured using any appropriate technology including, but not limited to, enteric coating, polymer coating, lipid coating, lipid blend coating, lipid encapsulation, direct compression, dry granulation, wet granulation, and any combination thereof. The one or more digestive enzyme(s) may be utilized with one or more existing, emergent, or future coating technologies. A variety of physical and chemical methods are then used to combine the drug with the coating material such as coacervation, emulsions, meltable dispersion, spray drying, pan coating and fluidized bed granulations. In this manner, coatings and digestive enzymes or other active pharmaceutical ingredients can be combined to control the kinetics, duration, time, control of peak concentration, pharmacokinetics, dose, and location of drug release in the body.

A composition may be an oral dosage formulation such as, for example, a suspension, solution, a slurry, pill, tablet (mini-tab, etc.), capsule (e.g., microcapsule, mini-capsule, time-released capsule, etc.), sprinkle, or any combination thereof. One or more of the compositions described herein may be administered to a subject.

In one embodiment, the digestive enzymes and the amount of each digestive enzyme present in such compositions may be empirically determined by a physician based upon the patient to be treated. A physician can readily determine and prescribe the effective amount of the composition required. For example, the physician could start doses of the compounds employed in the composition at levels lower than that required in order to achieve the desired therapeutic effect and gradually increase the dosage until the desired effect is achieved. Alternatively, a dose can remain constant.

### Coatings

The nature of the human digestive tract creates challenges for the delivery of digestive enzymes to subjects. Multiple temperature and pH changes over the course of the digestive tract make specific delivery a challenge but a necessity. For instance, pH as low as 1 is encountered in the stomach, but rapidly increases to a more basic pH of 5-6 in the proximal small intestine. For example, generally the pH in the stomach is approximately 1.2, the pH in the duodenum is about 5.0 to 6.0; the pH in the jejunum is about 6.8, and the pH is about 7.2 in the proximal ileum and about 7.5 in the distal ileum. The low pH in the stomach that changes rapidly to a more basic pH of 5-6 in the proximal small intestines calls for a specific delivery method depending upon where the enzyme is to be delivered.

In one example, the release of digestive enzymes is timed to release specific percentages of enzymes in specific portions of the gastrointestinal tract by use of coating technologies. Administration of the digestive enzymes described herein via any available route increases a concentration of the digestive enzymes in the subject such that they are available to, for example, cleave one or more spike protein(s) of a coronavirus.

A coating can, in some instances, provide a significant barrier to moisture, heat, humidity and exposure to light by allowing for a physical barrier as well as one that prevents and or reduces hydrolysis. Coated enzyme preparations undergo less hydrolysis as a result of protection from moisture in the environment by the lipid coating. As a result, digestive enzymes are provided which can tolerate storage conditions (e.g., moisture, heat, oxygen, etc.) for long periods of time thus enabling extended shelf life. The coating protects the digestive enzymes from an environment and can provide, in some instances, emulsification in a solvent without detracting from the abrasion resistance of the coating. The coated digestive enzyme preparations, therefore, reduce overfilling of the digestive enzyme dosage and enhance delivery of more accurate doses of the digestive enzymes.

In some instances, coating may be used to obtain release of digestive enzymes from a pharmaceutical composition or formulation at selected transit times or in selected locations of the gastrointestinal tract of humans. In one aspect, this disclosure relates to controlled-release enzyme preparations administered to a subject with a coronavirus infection (e.g., COVID-19). In another aspect of the present invention, one or more coating(s) are utilized to target delivery to the small intestines.

Some embodiments utilize stable enzyme preparations protected against the environment to reduce, for example, degradation and/or denaturation of the enzymes. This permits delivery of more accurate doses of the digestive enzymes to subjects needing treatment. The coating can also, in some aspects, provide emulsification when the enzyme preparations are contacted with appropriate solvents, while also surprisingly providing for controlled-release of the enzyme in the GI system. The emulsification properties of the coating in a solvent allows for controlled-release of the digestive enzymes at, for example, in selected locations in the GI tract or in the mucosa of a subject.

A pharmaceutical composition described herein, in one instance, comprises coated particles that contain (a) a core and (b) a coating. In some embodiments, a coated enzyme preparation is in the form of coated particles, which coated particles comprise: (a) a core comprising digestive enzymes present in the coated particles in an amount of from about 5% to 95% by weight and (b) a coating. In one aspect, this disclosure relates to an enzyme delivery system comprising a coated enzyme preparation in the form of coated particles, which coated particles comprise: (a) a core comprising digestive enzymes present in the coated particles in an amount of from about 50% to about 95% by weight; and (b) a coating. In yet another aspect, this disclosure relates to an enzyme delivery system comprising a coated enzyme preparation having coated particles, which coated particles comprise: (a) a core comprising digestive enzymes present in the coated particles in an amount of from about 70% to about 90% by weight; and (b) a coating. In yet another aspect, this disclosure relates to an enzyme delivery system comprising a coated enzyme preparation having coated particles, which coated particles comprise: (a) a core comprising digestive enzymes present in the coated particles an amount of from about 75% to about 85% by weight; and (b) a coating. The coated particles may be non-aerosolizable.

The core comprises one or more digestive enzyme(s) (e.g., proteases, lipases, amylases, or a combination thereof). Any suitable coating for use in the pharmaceutical compositions described herein includes, but is not limited to, a lipid, a mixture of lipids, a lipid blend, or a polymer enteric coating as described in more detail below. In one non-limiting aspect, coatings in pharmaceutical compositions create a barrier to degradation and denaturation and allow more accurate levels of active enzymes to reach the treated subjects.

A pharmaceutical composition described herein, in one instance, comprises coated particles that contain (a) a core and (b) a lipid coating. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a lipid coating. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a lipid coating. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a lipid coating. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a lipid coating. The coated particles may be non-aerosolizable.

A pharmaceutical composition described herein, in one instance, comprises coated particles that contain (a) a core and (b) a lipid coating, wherein the lipid comprises a monoglyceride, a diglyceride, a triglyceride, or a combination thereof. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a lipid coating, wherein the lipid comprises a monoglyceride, a diglyceride, a triglyceride, or a combination thereof. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a lipid coating, wherein the lipid comprises a monoglyceride, a diglyceride, a triglyceride, or a combination thereof. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a lipid coating, wherein the lipid comprises a monoglyceride, a diglyceride, a triglyceride, or a combination thereof. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a lipid coating, wherein the lipid comprises a monoglyceride, a diglyceride, a triglyceride, or a combination thereof. The coated particles may be non-aerosolizable.

A pharmaceutical composition described herein, in one instance, comprises coated particles that contain (a) a core and (b) a coating comprising a hydrogenated soy oil. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a coating comprising a hydrogenated soy oil. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a coating comprising a hydrogenated soy oil. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a coating comprising a hydrogenated soy oil. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a coating comprising a hydrogenated soy oil. The coated particles may be non-aerosolizable.

A pharmaceutical composition described herein, in one instance, comprises coated particles that contain (a) a core and (b) a coating comprising a mixture of lipids. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a coating comprising a mixture of lipids. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a coating comprising a mixture of lipids. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a coating comprising a mixture of lipids. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a coating comprising a mixture of lipids. The coated particles may be non-aerosolizable.

A pharmaceutical composition described herein, in one instance, comprises coated particles that contain (a) a core and (b) a coating comprising a lipid blend. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a coating comprising a lipid blend. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a coating comprising a lipid blend. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a coating comprising a lipid blend. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a coating comprising a lipid blend. The coated particles may be non-aerosolizable.

A pharmaceutical composition described herein, in one instance, comprises coated particles that contain (a) a core and (b) a polymer enteric coating. In some embodiments, a coated enzyme preparation is in the form of coated particles, which coated particles comprise: (a) a core comprising digestive enzymes (e.g., protease, amylase, and lipase) present in the coated particles in an amount of from about 5% to 95% by weight and (b) a polymer enteric coating. In one aspect, this disclosure relates to an enzyme delivery system comprising a coated enzyme preparation in the form of coated particles, which coated particles comprise: (a) a core comprising digestive enzymes (e.g., protease, amylase, and lipase) present in the coated particles in an amount of from about 50% to about 95% by weight; and (b) a polymer enteric coating. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles in an amount of from about 70% to about 90% by weight; and (b) a polymer enteric coating. In yet another aspect, this disclosure relates to an enzyme delivery system comprising a coated enzyme preparation having coated particles, which coated particles comprise: (a) a core comprising digestive enzymes (e.g., protease, amylase, and lipase) present in the coated particles an amount of from about 75% to about 85% by weight; and (b) a polymer enteric coating.

Pharmaceutical compositions (preparations) comprising uncoated or coated digestive enzymes may be manufactured using any appropriate technology including, but not limited to, polymer enteric coating, lipid encapsulation, lipid coating, wax coating, direct compression, dry granulation, wet granulation, and any combination thereof. In one embodiment, one or more digestive enzyme(s) are utilized with one or more existing, emergent, or future coating technologies. Coating technology selection is based upon one or more desired parameters including, but not limited to, desired release kinetics, duration, control of peak concentration, pharmacokinetics, and dose.

The property of physical and chemical methods may be used to combine the digestive enzymes with a coating material such as coacervation, emulsions, meltable dispersion, spray drying, pan coating, and fluidized bed granulations. In this manner, coatings and digestive enzymes can be combined to control the kinetics, time and location of enzyme release in the body. In one non-limiting instance, about 80% of the digestive enzymes are released by about 30 minutes after the coated digestive enzyme particles reach the small intestine.

As described herein, suitable digestive enzymes and optional suitable coatings may be used in the compositions and methods of this invention. The choice of suitable digestive enzymes and of suitable coatings, including choice of the type or the amounts of digestive enzymes and/or coating, are guided by the specific digestive enzyme needs of the subject, and the selected diseases to be treated.

Delivery of digestive enzymes can also be challenging due to the rapid degradation and denaturing of enzymes at ambient room temperature, as well as the enhanced degradation and denaturing that can occur with high temperature, pressure, humidity and/or exposure to light. Moisture and heat together can quickly destabilize enzymes, reducing their effectiveness, and shortening shelf life, leading to inaccurate dosing. Denaturization or destabilization of the digestive enzymes can reduce their effectiveness by reducing the dose of active enzymes to less than the amount needed for effective treatment. Alternatively, attempting to compensate for the denaturization or destabilization by increasing the dose to ensure an effective level of active enzyme, could risk an overdose or overfilling a capsule or other dosage form.

Coatings can be used to help mitigate these factors. In one embodiment, coating of a digestive enzyme preparation is used to obtain release at selected transit times or in selected locations of the gastrointestinal tract of humans. In one aspect, this relates to controlled-release enzyme preparations administered to a subject with a coronavirus infection (e.g., COVID-19). In another aspect, one or more coatings are utilized to target delivery to the small intestines.

In addition, a coating can provide controlled-release of digestive enzymes in some instances. For example, the emulsification properties of a coating in a solvent allows for controlled-release of the enzyme in the gastrointestinal system, such as the region of the GI tract where the digestive enzymes are to be utilized after administration. The coating protects the enzyme from the environment and provides emulsification in a solvent (e.g., a biological fluid) without detracting from the abrasion resistance of the coating. For example, the release of the protease from a pharmaceutical composition may occur in the proximal small intestine, and the coating has a dissolution profile between 30-90 minutes with 80% or greater release. Lower levels of release are still beneficial and may be utilized in some embodiments. The dissolution profile may also be about 30, 35, 40, 45, 50, 55, 60, 65, 70, 75, 80, 85, or 90 minutes. Dissolution profiles may be obtained using methods and conditions known to those in the art. For example, dissolution profiles can be determined at various pHs, including pHs 1, 2, 3, 4, 5, 6, 7, 8, 9 and 10.

“Encapsulate” as used herein means that the coating completely surrounds the digestive enzyme. In the manufacture of pharmaceutical compositions, encapsulation refers to a range of techniques used to enclose medicines in a relatively stable shell known as a capsule, allowing them to, for example, be taken orally or be used as suppositories.

### Lipid Coatings

Lipid coating or encapsulation may reduce aerosolization of digestive enzymes, which may be caustic to a subject if inhaled. For example, lipid encapsulation reduces aerosolization and the potential for caustic burn, aspiration, and/or aspiration pneumonias in subjects to be treated and administrators of the pharmaceutical composition, thereby reducing the potential for illness in already immunocompromised patients with a coronavirus infection, thereby leading to safer administration.

“Lipids”, as used herein, refers to lipids which contain at least one hydrophilic group and at least one hydrophobic group, and have a structure capable of forming a hydrophilic and hydrophobic interface. These chemical and/or physical properties, mentioned above, of a lipid permit emulsification. Examples of interfaces include, for example, micelles and bilayers. The hydrophilic group can be a polar group and can be charged or uncharged.

Digestive enzymes obtained from a pig can possess a significant odor and/or taste similar to cured or smoked pork. This taste and smell can be strong and offensive to some subjects, such as children. In one embodiment, the addition of a lipid coating significantly masks odor and taste of digestive enzymes enzyme, which allows for the tolerance of taste as the lipid coating is odorless and tasteless. The coated digestive enzyme compositions described herein may have improved taste and odor compared to non-coated digestive enzymes.

The lipid may be any lipid, lipid mixture, or blend of lipid of lipid with emulsifiers which emulsifies when exposed to a solvent and has a melting point which allows the lipid to be a solid at typical storage temperatures and/or additives. In some embodiments, the lipid consists essentially of or comprises one or more monoglycerides, diglycerides or triglycerides, or other components including, for example, emulsifiers found in hydrogenated vegetable oils. In another embodiment the lipid is a non-polar lipid. Examples of lipids include, but are not limited to, monoglycerides, diglycerides, triglycerides, fatty acids, esters of fatty acids, phospholipids, salts thereof, and combinations thereof. In one non-limiting instance, the lipid coating comprises monoglycerides, diglycerides, triglycerides, or a combination thereof. In another non-limiting instance, the lipid coating comprises monoglycerides, diglycerides, or triglycerides. In another non-limiting instance, the lipid coating comprises a combination of monoglycerides and diglycerides. In another non-limiting instance, the lipid coating comprises a combination of diglycerides and triglycerides. In another non-limiting instance, the lipid coating comprises a combination of monoglycerides, diglycerides, and triglycerides.

In another embodiment with respect to coated particles which contain a core and a coating, the enzymatic core containing a protease, a lipase, and an amylase, is coated by an inert lipid that allows for safe delivery of the enzymatic core through the oropharynx and into the stomach where it is quickly and efficiently released in the stomach and duodenum. The enzymatic core thereby destroys the coronavirus (e.g., SARS-CoV-2) virus early in the GI tract before major sites of ACE2 expression are reached in the late portion of the gut. For nasal, rectal, parenteral, percutaneous endoscopic gastrostomy (PEG), esophagogastroduodenoscopy (EGD), gastrostomy (G-tube) insertion, intravenous, etc., types of administration, digestive enzymes are absorbed into the body and made available to cleave spike proteins on circulating coronaviruses.

The lipid can be a vegetable-derived lipid or an animal-derived lipid. As used herein, animal lipids and/or vegetable lipids include, but are not limited to, fats and oils originating from plant sources, animal sources and/or tissues, and/or synthetically produced based on the structures of fats and oils originating from plant or animal sources. Lipid material may be refined, extracted or purified by known chemical or mechanical processes. Certain fatty acids present in lipids, termed essential fatty acids, must be present in the mammalian diet. The lipid may, in some embodiments, comprise a Type I US Pharmacopeia (U.S.P.) National Formulary vegetable oil. The lipid can be derived from animal origins or vegetable origins, such as, for example, palm kernel oil, soybean oil, cottonseed oil, canola oil, or poultry fat, including hydrogenated type I vegetable oils. In some embodiments, the lipid is hydrogenated. The lipid can also be saturated or partially saturated.

The rate of release of the digestive enzyme from a pharmaceutical composition upon exposure to a solvent (e.g., stomach acid) may be controlled by coating digestive enzymes with a lipid to form coated particles that comprise a core (which contains the digestive enzymes), and a coating which contains the lipid. Alternatively, the rate of release of the digestive enzyme from a pharmaceutical composition upon exposure to a solvent (e.g., stomach acid) may be controlled by (i) blending a lipid with an amount of one or more additives to obtain a lipid blend; and (ii) coating digestive enzymes with the lipid blend to form coated particles that comprise a core (which contains the digestive enzymes), and a coating which contains the lipid blend. In one instance, the coating comprises a lipid blend where the lipid and additive are not the same, and the rate of release of the digestive enzymes from the coated particles upon exposure to a solvent is decreased as the amount of additive is increased. In the alternative, the rate of release of the digestive enzymes from the coated particles upon exposure to a solvent is increased as the amount of additive is increased.

The lipid coating surprisingly does not appear to be reduced or destroyed by hydrochloric acid (HCl) present in the stomach, thereby protecting the digestive enzymes from degradation following administration until the digestive enzymes reach their target region in the proximal GI tract. Further, a lipid coating may reduce the exposure of the digestive enzymes to attack by water, thereby reducing hydrolysis, and further protecting the digestive enzymes from degradation. In addition, the inventors have found that a coating containing only lipid can be used to coat digestive enzymes that only contain one or more lipase(s).

Because, in some embodiments, a lipid coating does not require the digestive enzymes to be treated with solvents, extenders and excipients to facilitate flow or improve stability, a lipid coating described herein includes a “clean” preparation of GRAS (generally regarded as safe) substances to be administered. The reduction in the use of solvents, extenders, excipients and other additives permitted by the methods described herein reduces the exposure of the individuals taking the digestive enzymes to potential allergens, thereby producing a hypoallergenic enzyme preparation that further enhances its potential uses in the treatment of individuals who might otherwise develop an allergic response to treatment. Administration of the coated digestive enzyme compositions described herein can thus reduce exposure to potentially toxic substances and will also reduce the possibility of allergy formation. Accordingly, in some embodiments, the pharmaceutical composition is hypoallergenic.

The lipid may be, in some instances, a “food grade lipid”. Examples of food grade lipids include, but are not limited to, a sorbitan monostearates, sorbitan tristearates, and calcium stearoyl lactylates. Other examples of food grade fatty acid esters which are lipids include acetic acid esters of mono- and diglycerides, citric acid esters of mono- and di-glycerides, lactic acid esters of mono- and di-glycerides, polyglycerol esters of fatty acids, propylene glycol esters of fatty acids, and diacetyl tartaric acid esters of mono- and diglycerides.

The lipid may also be, in some instances, a “pharmaceutical grade lipid”. Pharmaceutical grade lipids include, but are not limited to, highly purified lipid from which all protein antigens have been removed. Such lipids are beneficial in that they do not induce allergic responses and do not include cis or trans fatty acids. One non-limiting example of a pharmaceutical grade lipid comprises a soybean oil that is fully hydrogenated. One non-limiting example of a pharmaceutical grade lipid consists of a soybean oil that is fully hydrogenated.

In some instances, a lipid coating will produce non-agglomerating, non-aerosolizing coated digestive enzyme particles.

The inclusion of one or more additives with a lipid can be used to control emulsification or dissolution of the coating and release of the digestive enzymes. For example, a triglyceride, can be blended with a monoglycerides to control emulsification or dissolution of the coating and thus control (e.g., decrease) the rate of release of the digestive enzymes from the coated particles. As a further example, a diglyceride and a triglyceride can be blended with a monoglyceride to control the rate of release of the digestive enzymes. Hydrogenated vegetable oils may contain emulsifying agents, such as soy lecithin or other components.

Properties including mechanical strength, melting point, and hydrophobicity can be considered when choosing a suitable lipid coating for the digestive enzymes. Lipids having lower melting points or more polar, hydrophilic properties are generally less suitable for the coating because they may result in a product that would cake under accelerated storage stability conditions. Coated digestive enzyme particles made using, for example, hydrogenated soy oil (e.g., partially or fully hydrogenated), which demonstrated good pouring and no caking.

In some embodiments a coated digestive enzyme preparation comprises coated particles, where the coated particles comprise (a) a core containing digestive enzymes; and (b) a coating comprising one or more lipids; where the digestive enzymes are present in the coated particles in an amount of from about 5% to about 95% by weight, including, but not limited to, about 5%, 10%, 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, and 95% by weight, along with all values/integers in-between. In some embodiments a coated digestive enzyme preparation comprises coated particles, where the coated particles comprise (a) a core containing digestive enzymes; and (b) a coating comprising a lipid blend; where the digestive enzymes are present in the coated particles in an amount of from about 5% to about 95% by weight, including, but not limited to, about 5%, 10%, 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, and 95% by weight, along with all values/integers in-between. In one embodiment, the coating continuously coats the core. In another embodiment, the lipid or the lipid blend releases the digestive enzymes upon exposure to physiological conditions.

In some embodiments, a coated enzyme preparation having particles which comprise: (a) a core comprising digestive enzymes present in an amount of from about 5% to 95% by weight of the particles, including 5%, 10%, 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90%, and 95% by weight along with all values/integers in-between; and (b) a generally uniform coating to provide for controlled-release of the digestive enzymes, the coating comprising a crystallizable lipid. Some coated digestive enzyme preparations which comprise a coating of a crystallizable lipid and a digestive enzyme core have favorable release and activity profiles and permit site time specific and/or location specific targeted release along the GI tract.

The methods of this disclosure can be used to produce coated digestive enzyme preparations comprising digestive enzymes coated with a crystallizable lipid alone, or with a lipid blend to achieve a controlled rate of enzyme release, with increased release of the digestive enzyme upon exposure of the coated preparation to a suitable solvent. Coated digestive enzyme preparations having a coating comprising one or more monoglycerides exhibit increased release of the digestive enzyme upon exposure of the coated composite to a solvent, such as water, while protecting against release in 0.1 N HCl.

In some embodiments, the compositions according to this disclosure produce enzyme preparations, including coated enzyme preparations, characterized, for example, by controlled rates of release, reduction in aerosolization and safer administration, ability to be administered by a sprinkle/sachet delivery method, improved flow characteristics, enhanced shelf life and storage capacity, and other properties described herein. In other aspects, the coated enzyme preparation has improved pour properties which facilitate manufacturing and packaging processes, for example packaging in pouches and sachet.

In some aspects, coated digestive enzyme preparations are prepared to obtain specific delivery times or specific regions within the GI tract. In some embodiments, the crystallizable lipid composition comprises, consists essentially of, or consists of, a hydrogenated soybean oil, but may be any suitable crystallizable lipid or lipid blend. Lipid coating or encapsulation reduces aerosolization of the digestive enzymes that may be caustic to a subject if inhaled through the lungs or the nose. In another embodiment, delivery of digestive enzymes with improved safety of administration can be achieved by reducing the amount of aerosolization of the enzyme. The lipid coating or encapsulation reduces aerosolization and the potential for caustic burn, aspiration, and/or aspiration pneumonias in subjects and administrators of the enzyme preparation, thereby reducing the potential for illness in already compromised patients such as those with a coronavirus infection (e.g., COVID-19), thereby leading to safer administration.

In one instance, the method relates to preparation of coated digestive enzyme particles that have enhanced flow properties and which are useful in the treatment of subjects with a coronavirus infection, the method comprising: a) blending a lipid with one or more additives to obtain a lipid blend; and b) coating screened digestive enzyme with the lipid blend to form the coated digestive enzyme particles containing a core which contains the digestive enzymes and a coating which contains the lipid blend.

### Waxes

Waxes may be utilized for coating digestive enzymes. In one embodiment an unreactive wax is utilized. The wax can be paraffin wax; a petroleum wax; a mineral wax such as ozokerite, ceresin, or montan wax; a vegetable wax such as, for example, carnauba wax, bayberry wax or flax wax; an animal wax such as, for example, spermaceti; or an insect wax such as beeswax. Additionally, the wax material can be an ester of a fatty acid having 12 to 31 carbon atoms and a fatty alcohol having 12 to 31 carbon atoms, the ester having from a carbon atom content of from 24 to 62, or a mixture thereof. Examples of waxes include, but are not limited to, myricyl palmitate, cetyl palmitate, myricyl cerotate, cetyl myristate, ceryl palmitate, ceryl certate, myricyl melissate, stearyl palmitate, stearyl myristate, and lauryl laurate. Digestive enzyme pharmaceutical compositions made using, for example, hydrogenated castor wax or carnauba wax may demonstrate good pouring and no caking.

### Polymer Enteric Coatings

In another aspect, digestive enzymes may be coated with a polymeric coating. The same principles of controlled dissolution still apply but access to a variety of materials expands options for control of both the location and rate of release. In one embodiment, one or more hydrophobic or hydrophilic polymers are used separately or in combination. Polymer enteric coatings for use in a composition described herein include, but are not limited to, cellulose acetate phthalate (CAP), cellulose acetate trimellitate (CAT), hydroxyl propyl methyl cellulose phthalate (HPMCP), hydroxyl propyl methyl cellulose acetate succinate (HPMCAS), ethyl cellulose (EC), polyvinyl acetate phthalate (PVAP), a methacrylic acid copolymer, a shellac (esters of aleurtic acid), Zein, ethylcellulose, gelatins, polyvinyl alcohol, styrene maleic anhydride, or a combination thereof. It should be noted that certain coatings which utilize cellulose (e.g., HPMC/HPEC, EUDRAGIT®, etc.) may be hydrophilic and, as such, absorb moisture which degrades the activity of digestive enzymes (e.g., pancreatin, etc.) utilized in various embodiments described herein.

In some embodiments, a pharmaceutical dosage form comprises a population of extended-release beads, wherein said extended-release beads comprise: an active-containing core particle comprising digestive enzymes as the active agent; and an extended-release coating comprising a water-insoluble polymer membrane surrounding said core, wherein said water-insoluble polymer membrane comprises a polymer selected from the group consisting of ethers of cellulose, esters of cellulose, cellulose acetate, ethyl cellulose, polyvinyl acetate, neutral copolymers based on ethyl acrylate and methyl methacrylate, copolymers of acrylic and methacrylic acid esters with quaternary ammonium groups, pH-insensitive ammonio methacrylic acid copolymers, and mixtures thereof; wherein the total amount of digestive enzymes in the pharmaceutical dosage form contain from about 15,000 U.S.P. Units protease to about 1.5 million U.S.P. Units protease per dose and where the ratio of protease to lipase is such that the amount of lipase is never more than 0.188 times the amount of protease and where the ratio of protease activity to amylase activity is between 1:0.1 and 1:10.

In some embodiments, an extended-release coating further comprises a water-soluble polymer selected from the group consisting of methylcellulose, hydroxypropylcellulose, hydroxypropyl methylcellulose, polyethylene glycol, polyvinylpyrrolidone, and mixtures thereof.

### Cellulose Acetate Phthalate (CAP)

Cellulose acetate phthalate, also known as cellacefate is a synthetic enteric coating polymer. Because the degree of substitution can lead to changes in CAP properties, specifications for CAP composition have been established to ensure more uniform performance from batch to batch. According to United State Pharmacopoeia (U.S.P.) specifications, CAP should contain from about 21.5% to about 26.0% w/w acetyl content and from about 30.0% to about 36.0% w/w phthalyl groups on the cellulose backbone as calculated on an anhydrous basis. CAP exhibits rapid dissolution at a pH >6 and is relatively permeable to moisture and gastric juices. Due to its high moisture permeability, CAP is susceptible to hydrolytic decomposition. Phthalic and acetic acid molecules may hydrolyze during storage and significantly compromise the degree of enteric protection that the film coating provides. The addition of a plasticizing agent can improve the water-resistance of CAP films. CAP is compatible with most water-soluble and insoluble plasticizers with diethyl phthalate (DEP), tributyl citrate (TBC), triethyl citrate (TEC), tributyrin, and triacetin being the most commonly used, typically, in the range of from about 25% to about 35% by weight of dry polymer. CAP is commercially available as a white powder from, for example, Eastman Chemical Co. A 30% solids latex dispersion version of CAP (AQUACOAT® CPD) is available for aqueous enteric coating of tablets, beads, and both hard and soft gelatin capsules.

### Cellulose Acetate Trimellitate (CAT)

Chemically cellulose acetate trimellitate (CAT) bears a strong resemblance to CAP. It is formed by the same synthesis process as CAP with trimellitic anhydride as the substituent group in place of phthalic anhydride. Typical values for timellityl and acetyl substitution are 29.0% and 22.4%, respectively. Trimellitic anhydride contains an additional free carboxyl group over that of phthalic anhydride, and hence CAT contains a greater concentration of acidic groups for a given degree of substitution than CAP rendering it more soluble in aqueous media. Also, the pKa of CAT is between 4.1 and 4.3 which is slightly lower than CAP. With a relatively low pKa value and greater functional group concentration, CAT is the most soluble enteric cellulose derivative with the onset of dissolution occurring at pH 4.7-5.0. This useful property makes CAT ideal for targeted drug release to the proximal regions of the small intestine. CAT is commercially available as a white powder from Eastman Chemical Co. To obtain the best enteric coating results from aqueous processing, ammoniacal solutions of CAT in water are recommended. Plasticizer considerations for CAT are identical to that of CAP.

### Hydroxyl Propyl Methyl Cellulose Phthalate (HPMCP)

HPMCP is a white to slightly off-white, free-flowing flakes or granular powder with a slightly acidic odor and a barely detectable taste. It is a derivative of hydroxypropyl methylcellulose that is produced by the transesterification of hydroxypropyl methylcellulose with phthalic acid and is a cellulose derivative for enteric coating. HPMCP has been admitted in the European and Japanese pharmacopoeias and included in the USP/NF under the name hypromellose phthalate. Depending on the degree of phthalyl substitution, HPMCP is soluble in aqueous media in a pH range of from about 5.0 to about 5.5. HPMCP is characteristically insoluble in gastric fluids but swellable and rapidly soluble in the upper intestine. It may be plasticized with diethylphthalate, acetylated monoglyceride or triacetin. Mechanically, HPMCP is a more flexible polymer and on a weight basis and will not require as much plasticizer as CAP or CAT. Solvent mixtures can be effectively prepared for commercial spray-drying by using proper spray-drying optimization.

### Hydroxyl Propyl Methyl Cellulose Acetate Succinate (HPMCAS)

HPMCAS, also known as hypromellose acetate succinate, is a white to off-white powder or granules derived from HPMC by the esterification of free hydroxyl groups on the polymer backbone with acetic anhydride and succinic anhydride. It is commercially available in three grades (L, M & H), which correspond to pH-dependent release profiles of low pH (5.0), medium (5.5), and high (6.5) pH. HPMCAS is soluble in neutral pH according to ionization of free carboxyl groups on the polymer backbone.

### Polyvinyl Acetate Phthalate (PVAP)

Polyvinyl acetate phthalate is a free-flowing white to off-white powder with a slight odor of acetic acid. The onset of aqueous dissolution of PVAP begins at a pH of about 5.0, allowing for enteric release as well as targeted drug release in the proximal small intestine. Although structurally similar to CAP (containing the dicarboxylic phthalic acid in a partially esterified form), PVAP is relatively more stable to hydrolysis than CAP due to its lower moisture permeability. It is compatible with plasticizers such as, for example, glyceryl triacetate, Triethyl citrate (TEC), acetyl triethylcitrate, Diethyl phthalate (DEP), and polyethylene glycol (PEG) 400. PVAP (SURETERIC®) is commercially available from COLORCON® as a complete preformulated coating system consisting of a powder blend of PVAP, plasticizers, and other functional ingredients intended for reconstitution in water for rapid coating dispersion production.

### Methacrylic Acid Copolymers

Methacrylic acid copolymers (EUDRAGIT®) are widely used for enteric coating applications as they contain free carboxylic acid groups that are ionized whenever the pH of the environment exceeds 5.5. Several different types of EUDRAGIT® polymers with enteric release capabilities are commercially available in a wide range of different physical forms (aqueous dispersion, organic solution, granules and powders): Methacrylic acid methylmethacrylate copolymers (EUDRAGIT® L and S), and methacrylic acid ethyl acrylate copolymer (EUDRAGIT® L30D) are coating polymers for enteric formulations which allow targeting of specific areas of the intestine.

### Shellac

Edible Shellac may be employed as a glazing agent on a pharmaceutical composition that comprises a pill. Because of its acidic properties (resisting stomach acids), shellac-coated pills may be used for a timed enteric or colonic release. Shellac provides an excellent barrier against water vapor penetration.

### Zein

Zein is a class of prolamine protein found in maize (corn) that is usually manufactured as a powder from corn gluten meal. Pure zein is clear, odorless, tasteless, hard, water-insoluble, and edible. Zein's properties make it usable in pharmaceutical compositions and may be used as a coating the digestive enzymes described herein. It is classified as Generally Recognized as Safe (GRAS) by the U.S. Food and Drug Administration. For use as a pharmaceutical coating, zein is all natural and requires less testing per the U.S.P. monographs.

### Ethylcellulose

Ethyl cellulose (ethylcellulose) is a derivative of cellulose in which some of the hydroxyl groups on the repeating glucose units are converted into ethyl ether groups and which may be used as a thin-film coating material for coating a pharmaceutical composition described herein. Food grade ethyl cellulose is a non-toxic film and thickener which is not water soluble.

### Optional Components of Coatings

Coatings are often utilized with a variety of excipients including, but not limited to, one or more plasticizers, colorants (e.g., dyes), solvents, flavors (e.g., sweeteners), surfactants, disintegrants, lubricants, preservatives, anti-microbials, or a combination thereof.

Plasticizers are relatively low molecular weight materials which may be added to film-coating formulations to modify the physical properties of polymers. Some film-coating polymers are amorphous, and as such, exhibit a reasonably well-defined glass transition temperature, Tg (a fundamental characteristic of polymers that has an effect on polymer properties that can also influence film formation, especially when using aqueous polymer dispersions). Common effects of plasticizers on the properties of thin film coatings include: reducing tensile strength, reducing elastic modulus, altering film adhesion (including increasing under optimal use conditions), increasing the viscosity of coating liquid (effect is greater as plasticizer molecular weight is increased), altering film permeability (depends of physiochemical; properties of plasticizer), and/or reducing the glass transition temperature Tg of film (magnitude of effect influenced by compatibility with polymer). Non-limiting examples of plasticizers commonly used in film coating processes include: a. Polyols, such as glycerol (glycerin), polyethylene glycols (PEG 200-6000 grades) and propylene glycol; b. Organic esters, such as Diethyl phthalate (DEP), Dibutyl phthalate (DBP), Dibutyl sebacate (DBS), Triethyl citrate (TEC), Acetyltriethyl citrate (ATEC), Acetyltributyl citrate (ATBC), Tributyl citrate (TBC), and Triacetin (glyceryl triacetate; TA); c. Oils/glycerides, such as fractionated coconut oil, castor oil, and distilled acetylated monoglycerides (AMG); or d. a combination thereof. In some embodiments, an extended-release coating further comprises a plasticizer selected from the group consisting of triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides, and mixtures thereof.

Colorants are included in many film-coating formulations to: a. improve product appearance and/or enable product identification; b. modify the gas permeability of a film; c. decrease the risk of counterfeiting the product; d. protect the active ingredient against light by optimizing the opacifying properties of pigments; or e. a combination thereof.

Either water-soluble colorants (known as dyes) or water-insoluble colorants (known as pigments) may be utilized. Water-insoluble colorants may be utilized in film-coating formulations based on exhibition of: a. better light stability; b. better opacity and covering power; c. optimizing moisture barrier properties of the applied film coatings; and/or d. do not suffer from the disadvantageous phenomenon of mottling (caused by solute migration) that can be observed with water-soluble colorants. The effects of colorants on thin films include, but again are not limited to: reducing tensile strength (effect may be minimized by effective pigment dispersion in film); increasing the elastic modulus; slightly increasing the viscosity of the coating liquid, reducing film permeability (unless critical pigment volume concentration CPCV is exceeded); and increasing hiding power (effect is dependent upon refractive index and light absorption characteristics of pigment). Colorants typically utilized in thin film coatings include, but are not limited to Water-soluble dyes (e.g., FD&C Yellow #5, FD&C Blue #2, etc.); FD&C Lakes such as FD&C Yellow #5 Lake, FD&C Blue #2 Lake, etc.); D&C Lakes (e.g., D&C Yellow #10 Lake, D&C Red #30 lake, etc.), Inorganic Pigments (e.g., Titanium Dioxide, Iron Oxides, etc.); and Natural Colorants (e.g., Riboflavin, Beta-carotene, Carmine lake, etc.), or a combination thereof.

Solvents may be used to dissolve or disperse coating materials and convey them to the surface of the tablet core. Common solvents used in film coating include, but are not limited to Water, Ketones such as Acetone; Alcohols such as Methanol, Ethanol, and Isopropanol; Esters such as Ethyl acetate and Ethyl lactate; and Chlorinated Hydrocarbons such as Methylene Chloride, 1:1:1: Trichloroethane, and Chloroform.

While polymers, plasticizers, colorants, and solvents constitute the major ingredients in film-coating formulations, other materials might be used occasionally in low concentrations for specific formulations. Flavors and sweeteners may be added to mask unpleasant odor of the digestive enzymes or to make them more, palatable. Surfactants or dissolution enhancers such as polyoxyethylene sorbitan derivatives may be added to i. emulsify water-insoluble plasticizers; ii. improve substrate wettability and enhance spreadability of the film during application; iii. stabilize suspensions; or iv. a combination thereof.

Additionally, some film coatings may also contain preservative/antimicrobials (e.g., carbamates, alkylisothiazolinone, benzothiazoles, etc.), adhesion enhancers (such as polydextrose, maltodextrin, and lactose), antifoaming agents (e.g., dimethylpolysiloxane), antioxidants (e.g., oximes, phenols, etc.), pore-forming agents (e.g., sucrose or sodium chloride with ethylcellulose-coated salicylic acid tablets) and waxes. In rare instances, the film coat itself may contain active drug substance. When used, all ingredients used in film-coating formulations will comply with relevant regulatory and pharmacopoeial requirements.

Suitable disintegrants include, for example, sodium starch glycolate, other starches such as pregelatinized starch, and celluloses. Suitable lubricants may be provided such as, for example, magnesium stearate, calcium stearate, tale, stearic acid, etc.

### Other Coatings

The embodiments described herein are not limited to the aforementioned coatings. By way of example, other hydrophobic polymers that can be used as coatings include, but are not limited to, various forms of acrylics, amides/imides, olefins, styrenes, vinyl acetates/vinyl esters, or a combination thereof. Any existing, emergent, or yet to be developed coating which meets the appropriate safety and applicable regulatory requirements while delivering the active ingredients to the appropriate place in the gastrointestinal tract may be utilized.

### API Carrier Suspensions

Utilization of nano-crystal API carrier suspensions utilizing compounds such as Hydroxypropyl cellulose (HPC), hydroxypropyl methylcellulose (HPMC), lecithin, and docusate sodium to create nanoparticles and other dispersions for improved solubility and interaction with target molecules. Nanocrystals are small particles of the API combined with an excipient “carrier” for dispersion. Typically, particles are from about 0.1 nm to about 1 nm (e.g., about 0.5 nm) in size. These are created using dissolution of the fine API particles in a carrier and subsequent utilization of precipitation and emulsification or the use of high-pressure homogenization, sonication and micro-fluidization.

Carriers used for dispersion include, but are not limited to, HPMC, HPC, and decussate sodium. Docusate sodium (bis(2-ethylhexyl) sulfosuccinate), also commonly called dioctyl sulfosuccinate (DOSS) is a long chain carbon polymer with the formula C₂₀H₃₇NaO₇S. Lecithin refers to a group of animal-derived fat like substances. Chemically, they are composed of mixtures of glycerophospholipids, phosphatidylcholine, phosphatidylethanolamine, phosphatidylinositol, and phosphatidylserine.

### Matrices

A pharmaceutical composition may be prepared in which an excipient provides a matrix to capture and protect the digestive enzymes before delivery. Pharmaceutical compositions may be prepared whereby the subject who takes the composition has a reduction in the number of capsules/tablets per dosage; e.g., the preparation is stabilized and may contain a therapeutically effective amount of a protease, an amylase, and/or a lipase. Compositions may include, for example, a stabilizing matrix consisting essentially of a solidified microcrystalline cellulose which captures and protects therapeutically effective amounts of digestive enzyme particles within the stabilizing matrix. This can be done, for example, through the use of what is known in the art as PROSOLV® technology.

PROSOLV® is a combination of excipients which allow for optimized flow, compaction and product uniformity. This technology allows for uniformity in this combination, as well as manufacturing a very small tablet which would be amenable for children. With PROSOLV® technology, the ingredients are not just blended, but are co-processed, which assures that equal particles are uniformly distributed, and these results are easily reproducible. This allows for stability and superb product quality.

Whether utilizing the PROSOLV® method or other methodology, the one or more digestive enzyme(s) will be formulated and manufactured such that the particles will be uniformly distributed and there will be no overage with respect to the amount of enzyme found in the preparation. Said new drug formulation can be found in, but is not limited to, formulations which include digestive enzymes with and without the utilization of the PROSOLV® technology.

Digestive enzymes may be combined with one of the patented PROSOLV® technologies, e.g.: PROSOLV® SMCC 50 or PROSOLV® SMCC 90, or other PROSOLV® technologies. When employing the PROSOLV® method, the silicified microcrystalline cellulose (SMCC) used in a pharmaceutical composition described herein may be any commercially available combination of microcrystalline cellulose granulated with colloidal silicon dioxide. The SMCC generally will be as described in Sherwood et al., Pharm. Tech., October 1998, 78-88 and U.S. Pat. No. 5,585,115, which are incorporated herein by reference with respect to PROSOLV® technology. SMCC can be obtained commercially from Edward Mendell Company, Inc., a subsidiary of Penwest Ltd., under the name PROSOLV® SMCC. There are different grades of SMCC available, with particle size being the differentiating property among the grades. For example, PROSOLV® SMCC 90 has a median particle size, by sieve analysis, in the region of 90 micrometers. PROSOLV® SMCC 50 has a median particle size, by sieve analysis, in the region of from about 40 to about 50 micrometers.

### Pharmaceutical Compositions

The compositions described herein can be administered either alone or in combination with one or more of a conventional pharmaceutical carrier, buffer, stabilizer, surfactant, filler, binder, sweetener, or the like. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site to a portion of the body. Each carrier is acceptable in the sense of being compatible with the other ingredients of the formulation and not injurious to a subject to whom it is administered. Nor should an acceptable carrier alter the specific activity of the subject compounds.

Acceptable carriers are physiologically acceptable to the administered patient and retain the therapeutic properties of the compounds with/in which it is administered. Acceptable carriers and their formulations are and generally described in, for example, Remington' pharmaceutical Sciences (18th Edition, ed. A. Gennaro, Mack Publishing Co., Easton, Pa. 1990). Two exemplary carriers are water and physiological saline.

Other acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN®, PLURONICS®, or polyethylene glycol (PEG).

Such other ingredients and the amounts to be used are within the knowledge of one in the art and are known in the pharmaceutical arts.

Optionally, a digestive enzyme composition is prepared without the use of extenders colorants, dyes, flow enhancers and other additives to reduce the potential for allergens and other sensitivity reactions.

Compositions comprising an effective amount of the digestive enzymes may be formulated for administration to a subject, or may be administered to a subject, via any conventional route including but not limited to oral, parenteral, intramuscular, intravenous, transmucosal, transdermal, nasal, rectal (e.g., via suppository), percutaneous endoscopic gastrostomy (PEG), esophagogastroduodenoscopy (EGD), gastrostomy (G-tube) insertion, or other method. Oral administration can be in the form of solution, suspension, slurry, pellet, capsule, caplet, beadlet, sprinkle, tablet, softgel, or other. Alternatively, the pharmaceutical compositions can also be prepared for parenteral use. Such compositions typically take the form of sterile isotonic solutions of the digestive enzymes according to standard pharmaceutical practice.

The term “unit dose” when used in reference to a pharmaceutical composition refers to physically discrete units suitable as unitary dosage for humans, each unit containing a predetermined quantity of active material calculated to produce the desired therapeutic effect of reduction of one or more symptoms associated with COVID-19 or reducing infectivity of a coronavirus (e.g., in the mucosa or gut) in a prophylactic manner.

Determining a dosage regimen of the compound is well within the purview of those in the art. By way of example, the dose levels may range from about 100 milligrams to about 10 grams as determined by weight of a subject. Further activity of the digestive enzymes may range from 100 units of activity to 1,500,000 units of activity per dose for amylases, lipases and proteases, respectively.

Different dosage forms have different benefits. Tablets and capsules are the most common dosage forms for oral administration due to ease of manufacture, packaging and administration. Different forms of tablets have been primarily devised to meet the needs of select populations while maintaining the integrity of the active pharmaceutical ingredient. Some populations, notably infants and young children, cannot swallow tablets or capsules, or find it difficult to do so. In these instances, a tablet that dissolves under the tongue, in the mouth, or in a specified liquid, or one that could be harmlessly chewed would be beneficial. Tablets may also be micro-coated and placed in a capsule for administration. A tablet is a mixture of active substances and excipients, usually in powder form, pressed or compacted into a solid. The excipients include binders, glidants (flow aids) and lubricants to ensure efficient tableting; disintegrants to ensure that the tablet breaks up in the digestive tract; sweeteners or flavors to mask the taste of bad-tasting active ingredients; and pigments to make uncoated tablets visually attractive. A coating (sugar, enteric or film) may be applied to hide the taste of the tablet's components, to make the tablet smoother and easier to swallow, and/or to make it more resistant to the environment, extending its shelf life. Tablets, in some instances, may be buffered (by potassium metaphosphate, potassium phosphate, monobasic sodium acetate, etc.) to combat change in pH.

Tablets may be delayed-release, sustained-release, extended-release, controlled-delivery, long-acting, orally-disintegrating or melts, among others, often denoting the pharmacokinetic profile of the active agent. A capsule-shaped tablet is a caplet. Some tablets may be taken sublingually or allowed to dissolve in the mouth. The principle behind sublingual administration is simple. When a chemical comes in contact with the mucous membrane beneath the tongue, or buccal mucosa, it diffuses through it. Because the connective tissue beneath the epithelium contains a profusion of capillaries, the substance then diffuses into them and enters the venous circulation. Troches are medicated lozenges designed to dissolve in the mouth. Soluble tablets dissolve on contact with the tongue. A dose of a composition described herein may be formulated for oral administered in an amount of, for example, about ½ tablet, about 1 tablet, about 1.5 tablets, about 2 tablets, about 2.5 tablets, about 3 tablets, about 3.5 tablets, or about 4 tablets.

In one non-limiting embodiment, a direct compression method may be used for the manufacture of a pharmaceutical tablet preparation including the steps of: (a) forming an active blend by blending an intimate admixture of silicified microcrystalline cellulose and a therapeutic agent comprising one or more digestive enzyme(s); (b) forming a color blend by blending an intimate admixture of one or more pharmaceutically acceptable dyes and silicified microcrystalline cellulose, if color is necessary; (c) combining the active blend, the color blend, and a disintegrant into a pre-blend; (d) adding a lubricant to the pre-blend to form a final blend; and (e) compressing the final blend to form a pharmaceutical tablet preparation or a mixture of time released microtabs or a time-released tablet.

Capsules that could be opened and their contents sprinkled over a small amount of food or in a liquid would also be beneficial. Any improvement that eases the administration of a necessary medication or lessens the antagonism associated with said administration, without compromising the effectiveness of the active pharmaceutical ingredient, is worthwhile.

In the manufacture of pharmaceuticals, encapsulation refers to a range of techniques used to enclose medicines in a relatively stable shell known as a capsule, allowing them to, for example, be taken orally or be used as suppositories. The two main types of capsules are hard-shelled capsules, which are normally used for dry, powdered ingredients, and soft-shelled capsules, primarily used for oils and for active ingredients that are dissolved or suspended in oil. Both of these classes of capsule are made both from gelatin and from plant-based gelling substances like carrageenans and modified forms of starch and cellulose, and the latter form is usually seamless. Capsules are made in two parts by dipping metal rods in molten gelatin solution. The capsules are supplied as closed units to the pharmaceutical manufacturer. Before use, the two halves are separated, the capsule is filled with powder (either by placing a compressed slug of powder into one half of the capsule, or by filling one half of the capsule with loose powder) and the other half of the capsule is pressed on. The advantage of inserting a slug of compressed powder is that control of weight variation is better, but the machinery involved is more complex.

Sprinkle capsules are a dosage form consisting of small beads or granules of an active drug contained in a capsule that can be readily administered by simply opening up the capsule and distributing the contents over something to be swallowed.

In one embodiment, a coated digestive enzyme preparation or an uncoated digestive enzyme preparation is housed in a sachet which allows for particular types of administration including, but not limited to, administration in food, drink, or direct administration into the oral cavity or directly into the GI system through a NG-tube, G-tube or other GI entrances. The use of a sachet delivery of enzymes has heretofore not been utilized in the enzyme preparations presently marketed. In one embodiment, the sachet represents a single unit dosage or multiple doses for a day. The sachet of a trilaminar pouch allows the enzyme or enzyme/lipid powder to remain stable and allows for ease of administration.

In addition, the encapsulation also provides controlled-release of the digestive enzyme. In one embodiment, the emulsification properties of the coating in a solvent allows for controlled-release of the enzyme in the gastrointestinal (GI) system, preferably the region of the GI tract where the digestive enzymes are to be utilized.

Other types of solid dosage forms such as thin strips, lollipops or gum bring a novel concept to the administration of medications. Aside from the obvious ease of administration from the viewpoint of the caregiver, there may be an added benefit. The administration of medication is oftentimes a private issue and the ability of a caregiver to provide a dose of medication in a seemingly matter-of-fact form may preserve that perception and instill in the user a mindset that views the administration as pleasant rather than monotonous or negative.

Liquid dosage forms also provide benefits of administration to infants and young children or anyone with compromised swallowing capability. Syrups, solutions and suspensions are easily swallowed. Unpleasant tastes can be masked by flavoring. An oral spray allows for the quick administration of a pre-measured dose of medication and supplies multiple metered doses in one handy device. With no need to aid swallowing (such as a glass of water, etc.) and the convenience of not having to rifle through a bottle of tablets, administration is simplified. A dose of a pharmaceutical composition described herein may be formulated for oral administered in an amount of, for example, about 3 teaspoons, about 2.75 teaspoons, about 2.5 teaspoons, about 2.25 teaspoons, about 2 teaspoons, about 1.75 teaspoons, about 1.5 teaspoons, about 1.25 teaspoons, about 1 teaspoon, about ½ teaspoon, about ¼ teaspoon, or about ⅛ teaspoon.

A slurry may be made when a dissolvable tablet containing a gelling agent is added to a liquid.

A suspension is a heterogeneous fluid containing solid particles that are sufficiently large for sedimentation. Usually, they must be larger than 1 micrometer. The internal phase (solid) is dispersed throughout the external phase (fluid) through mechanical agitation, with the use of certain excipients or suspending agents. Unlike colloids, suspensions will eventually settle. An example of a suspension would be sand in water. The suspended particles are visible under a microscope and will settle over time if left undisturbed. This distinguishes a suspension from a colloid in which the suspended particles are smaller and do not settle. Colloids and suspensions are different from a solution, in which the dissolved substance (solute) does not exist as a solid and solvent and solute are homogeneously mixed. Oftentimes, powders of active ingredients may be packaged such that the addition of a diluent dissolves the powder and holds it in a liquid suspension.

When used as a pharmaceutical preparation, elixirs contain an active ingredient that is dissolved in a solution that contains some percentage (usually 40-60%) of ethyl alcohol and is designed to be taken orally.

Syrups are oftentimes employed as a base for medicinal purposes and consist of a concentrated or saturated solution of refined sugar in distilled water.

A suspension of liquid droplets or fine solid particles in a gas is called an aerosol. This can take the form of an oral spray, an oral rinse, a nasal spray, a nasal drop, etc.

A gum may be devised whereby an active ingredient is incorporated into a vegetative resinous substance (e.g. acacia) and released via the actual mechanical effect of chewing or the action of saliva on the gum itself.

A thinstrip is an active pharmaceutical product coated by a lipid layer designed to dissolve in the mouth over a brief period of time. The same technology could be used to produce a medicated lollipop for transmucosal delivery.

In pharmaceutical terms, a granule is a small particle gathered into a larger, permanent aggregate in which the original particles can still be identified.

Methods of preparing dosage forms are known, or will be apparent, to in this art; for example, Remington: The Science and Practice of Pharmacy, 21st Ed. (Lippincott Williams & Wilkins. 2005). Appropriate dosages will depend on the patient (age, weight, overall health, etc.), the severity of the condition, the type of formulation and other factors known to those having ordinary skill in the art. It is to be noted that concentrations and dosage values can vary with the severity of the condition. It is to be further understood that for any particular patient, specific dosage regimens should be adjusted over time according to the individual need and the professional judgment of the person administering or supervising the administration of the compositions.

Representative dosages for each of the combination formulations is presented below by drug, disease, and appropriate age category. In addition, dosages are provided for those who are renally or hepatically impaired. It should be noted that the use of these compositions in pregnancy may have risks as identified by the FDA Pregnancy Category Guidelines. Specifically, omeprazole and omeprazole/sodium bicarbonate, as well as bismuth subsalicylate are designated as Pregnancy Category C. All others listed below are designated Pregnancy Category B.

A pharmaceutical composition described herein may be prepared using a direct compression method, a dry granulation method, or by wet granulation. Preferably, the digestive enzyme preparation may be prepared using a direct compression process. This preferred process consists of two main steps: blending and compression. The blending step is composed of an active blend, color blend, pre-blend, and final blend (lubrication). A formulation may include a number of other ingredients for optimal characteristics of the pharmaceutical composition.

The rate of release of digestive enzymes can also be controlled by the addition of one or more additives. For example, when a pharmaceutical composition is exposed to a solvent, the solvent interacts with the coating and results in emulsification of the coating and release of the digestive enzymes.

In one embodiment, packaging of a pharmaceutical composition comprises single dose sachet-housed sprinkle preparations that allow for ease of delivery and accurate dosing of the digestive enzymes by allowing a specific amount of digestive enzymes to be delivered in each dosing. Allowing for specific unit dosing of digestive enzymes which maintains the enzyme activity within specific stability parameters is an enhancement over other sprinkle formulations, which are housed in a multi-unit dosing form that allows for air, moisture and heat to depredate and denature the enzyme preparation. In one embodiment, a powder or sachet is housed in a trilaminar pouch of which one layer is foil, or similar barrier to keep out moisture and to protect the enzyme preparation from adverse environmental factors.

A pharmaceutical composition can be administered to a subject one (1) or more times a day, such as for example, 1, 2, 3, 4, 5, 6, 7, 8, 9, or 10 times per day. In one instance, a composition can be administered orally 1, 2, or 3 times a day. Alternatively, a pharmaceutical composition can be administered to a subject, for example, 1, 2, 3, 4, 5, 6, 7, 8, 9, or 10 times per week. Alternatively, a pharmaceutical composition can be administered to a subject, for example, 1, 2, 3, 4, 5, 6, 7, 8, 9, or 10 times per month. In some instances, a subject is administered 1, 2, 3, 4, 5, 6, 7, 8, 9, or 10 doses at each treatment time. Administration can be with or without food.

### Representative Coated Compositions

In one aspect, a pharmaceutical composition comprises coated particles comprising (i) a core that comprises digestive enzymes, wherein the digestive enzymes comprise a protease, an amylase, a lipase, or a combination thereof; and (ii) a polymer coating. In some instances, the polymer coating comprises a polymer enteric coating.

In one aspect, a pharmaceutical composition comprises coated particles comprising (i) a core that comprises digestive enzymes, wherein the digestive enzymes comprise a protease, an amylase, a lipase, or a combination thereof; and (ii) an enteric coating.

In one aspect, a pharmaceutical composition comprises coated particles comprising (i) a core that comprises digestive enzymes, wherein the digestive enzymes comprise a protease, an amylase, a lipase, or a combination thereof; and (ii) a polymer coating. In some instances, the polymer coating comprises a polymer enteric coating.

In one aspect, a pharmaceutical composition comprises coated particles comprising (i) a core that comprises digestive enzymes, wherein the digestive enzymes comprise a protease, an amylase, a lipase, or a combination thereof; and (ii) a lipid coating.

The minimum amount of digestive enzymes present in the coated particles is about 5% active enzymes by weight. The maximum amount of digestive enzymes present in the coated particles is about 99% by weight, and in other embodiments at most about 90%, 85%, 80%, 75% or 70% of the coated enzyme preparation. In other embodiments, the amount of digestive enzymes present in the composite is about 5%, 6%, 7%, 8%, 9%, 10%, 15%, 20%, 25%, 35%, 40%, 45%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92.5%, 95%, or 99% by weight or anywhere in between. In other embodiments, the amount of digestive enzymes present in the composite is about 70%, 71%, 72%, 73%, 74%, 75%, 76%, 77%, 78%, 79%, 80%, 81%, 82%, 83%, 84%, 85%, 86%, 87%, 88%, 89%, or 90% by weight or anywhere in between. In other embodiments, the amount of digestive enzymes present in the composite is about 75%, 76%, 77%, 78%, 79%, 80%, 81%, 82%, 83%, 84%, or 85% by weight or anywhere in between. At least about or at most about a % of enzyme may include equal to or about that % of enzyme.

In some embodiments, a pharmaceutical composition comprises a coated particles which comprise: (a) a core comprising digestive enzymes present in an amount of from about 5% to about 95% by weight of the coated particles, including 5%, 6%, 7%, 8%, 9%, 10%, 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92.5%, 95%%, or 99% by weight along with all values in-between; and (b) a coating, the coating comprising a crystallizable lipid. In some embodiments, a pharmaceutical composition comprises a coated particles which comprise: (a) a core comprising digestive enzymes present in an amount of from about 70% to about 90% by weight of the coated particles, including 70%, 71%, 72%, 73%, 74%, 75%, 76%, 77%, 78%, 79%, 80%, 81%, 82%, 83%, 84%, 85%, 86%, 87%, 88%, 89%, or 90% by weight along with all values in-between; and (b) a coating, the coating comprising a crystallizable lipid. In some embodiments, a pharmaceutical composition comprises a coated particles which comprise: (a) a core comprising digestive enzymes present in an amount of from about 75% to about 85% by weight of the coated particles, including 75%, 76%, 77%, 78%, 79%, 80%, 81%, 82%, 83%, 84%, or 85% by weight along with all values in-between; and (b) a coating, the coating comprising a crystallizable lipid. In some embodiments, the coated enzyme preparation particles of the enzyme delivery system are non-aerosolizable. In some instances, the coating is generally uniform and provides for controlled-release of the digestive enzymes when administered to a subject.

In some embodiments a pharmaceutical dosage comprising a population of extended-release beads, wherein said extended-release beads comprise: an active-containing core particle comprising digestive enzymes as the active agent; and an extended-release coating comprising a water-insoluble polymer membrane surrounding said core, wherein said water-insoluble polymer membrane comprises a polymer selected from the group consisting of ethers of cellulose, esters of cellulose, cellulose acetate, ethyl cellulose, polyvinyl acetate, neutral copolymers based on ethyl acrylate and methyl methacrylate, copolymers of acrylic and methacrylic acid esters with quaternary ammonium groups, pH-insensitive ammonio methacrylic acid copolymers, and mixtures thereof; wherein the total amount of pancreatin in the pharmaceutical dosage form contains from about 15,000 U.S.P. Units protease to about 1.5 million U.S.P. Units protease per dose and where the ratio of protease to lipase in U.S.P. Units is such that the amount of lipase in the composition is never more than 0.188 times the amount of protease, and where the ratio of protease activity to amylase activity in U.S.P. Units is between 1:0.1 and 1:10.

In some embodiments the extended-release coating further comprises a water-soluble polymer selected from the group consisting of methylcellulose, hydroxypropylcellulose, hydroxypropyl methylcellulose, polyethylene glycol, polyvinylpyrrolidone and mixtures thereof.

In some embodiments the extended-release coating further comprises a plasticizer selected from the group consisting of triacetin, tributyl citrate, triethyl citrate, acetyl tri-n-butyl citrate, diethyl phthalate, dibutyl sebacate, polyethylene glycol, polypropylene glycol, castor oil, acetylated mono- and di-glycerides, and mixtures thereof.

In some embodiments, the pharmaceutical compositions are characterized, for example, by controlled rates of release, reduction in aerosolization and safer administration, ability to be administered by a sprinkle/sachet delivery method, improved flow characteristics, enhanced shelf life and storage capacity, and other properties described herein. In other aspects, the coated enzyme preparation has improved pour properties which facilitate manufacturing and packaging processes, for example packaging in pouches and sachet.

Some coated digestive enzyme preparations which comprise a coating of a crystallizable lipid and a digestive enzyme core have favorable release and activity profiles and permit site time specific and/or location specific targeted release along the GI tract. In some aspects, the coated digestive enzyme preparations are prepared to obtain specific delivery times or specific regions within the human GI tract. In some embodiments, the crystallizable lipid composition is hydrogenated soybean oil, but may be any suitable crystallizable lipid or lipid blend. Additionally, the coating of the coated digestive enzyme preparations may be tailored for optimal targeted release of the enzyme(s) to achieve maximal combined efficacy of the digestive enzymes when used in conjunction with acid reducer(s).

Some embodiments utilize stable enzyme preparations protected against the environment to reduce, for example, degradation and/or denaturation of the enzymes. This permits delivery of more accurate doses of the enzyme preparation to treated subjects. The coating can also, in some aspects, provide emulsification when the enzyme preparations are contacted with appropriate solvents, while also surprisingly providing for controlled-release of the enzyme in the GI system. The emulsification properties of the coating in a solvent allows for controlled-release of the enzyme, preferably at selected locations in the GI tract, where enzyme utilization provides the most effective prophylaxis or treatment.

In one embodiment, the digestive enzyme used present as consisting of particles having various sizes. In another embodiment, the particles of digestive enzyme are screened to obtain particles of a suitable size for encapsulation by removing particles that are too fine or too large. For example, the particles may be sieved to obtain particles of a suitable size or more uniform size range for encapsulation.

Compositions contemplated herein include, but are not limited to, solutions, suspensions, suppositories, pellets, capsules, caplets, beadlets, sprinkles, tablets, softgels, sachets, pouches, etc.

The minimum amount of digestive enzymes present in the coated particles is at least about 5% by weight. In one embodiment, the minimum amount of digestive enzymes present in the coated particles is at least about 30%, about 35%, about 40%, or about 50% by weight. The maximum amount of digestive enzymes present in the coated particles is about 99% by weight. For example, the amount of digestive enzymes present in the coated particles is at most 99%, about 98%, about 95%, about 90%, about 87.5%, about 85%, about 82.5%, about 80%, about 77.5%, about 75%, about 72.5%, or about 70% by weight.

In one instance, the amount of digestive enzymes present in the coated particles is about 5%, 10%, 15%, 20%, 25%, 35%, 40%, 45%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92.5%, or 95% by weight, or any integer in-between. “At least about” or “at most about” a percentage (%) of enzyme may include equal to or about that % of enzyme.

The coating can be present in coated digestive enzyme particles in an amount of from about 1% to about 50%, from about 1% to about 30%, or about 20%. The coating can be present in coated digestive enzyme particles in an amount of about 1%, about 2%, about 3%, about 4%, about 5%, about 6%, about 7%, about 8%, about 9%, about 10%, about 11%, about 12%, about 13%, about 14%, about 15%, about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 23.5%, about 24%, about 24.5%, about 25%, about 25.5%, about 26%, about 26.5%, about 27%, about 27.5%, about 28%, about 28.5%, about 29%, about 29.5%, about 30%, about 30.5%, about 31%, about 31.5%, about 32%, about 32.5%, about 33%, about 33.5%, about 34%, about 34.5%, about 35%, about 35.5%, about 36%, about 36.5%, about 37%, about 37.5%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 46%, about 46.5%, about 47%, about 47.5%, about 48%, about 48.5%, about 49%, about 49.5%, or about 50%, by weight. In some instances, the coating can be present in coated digestive enzyme particles in an amount of from about 10% to about 30% by weight. In some instances, the coating can be present in coated digestive enzyme particles in an amount of from about 15% to about 25% by weight. In some instances, the coating can be present in coated digestive enzyme particles in an amount of about 20% by weight.

In another embodiment enzyme preparations are utilized with lipid coating of enzymes. The method of making the preparations comprises providing a crystallizable lipid, and coating size-specific digestive enzyme particles as described herein with a lipid. The digestive enzymes may be present in the coated particles in an amount of from about 5% to about 95% by weight, including, but not limited to, about 5%, 10%, 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92.5%, or 95% along with all values in-between. The digestive enzymes may be present in the coated particles in an amount of from about 70% to about 90% by weight. The digestive enzymes may be present in the coated particles in an amount of from about 72.5% to about 87.5% by weight. The digestive enzymes may be present in the coated particles in an amount of from about 75% to about 85% by weight. The digestive enzymes may be present in the coated particles in an amount of from about 77.5% to about 82.5% by weight. The digestive enzymes may be present in the coated particles in an amount of about 80%.

In another example, the coatings and digestive enzymes are concentrically nested to allow timed release of enzymes in more than one portion of the gastrointestinal tract to prophylaxis or treat an infection or the symptoms thereof treat the symptoms thereof for one or more coronavirus(es).

The digestive enzymes can be used in cores of coated particles where about 90% of the coated particles are from about #40 to about #140 USSS mesh in size, or from about 105 to about 425 μm in size (diameter). Alternatively, the digestive enzyme particles used in cores of coated particles where about 75% of the particles are from about #40 to about #80 USSS mesh, or from about 180 to about 425 μm in size (diameter). Particles from about #40 to about #140 USSS mesh in size pass through #40 USSS mesh but do not pass through #140 USSS mesh. The coated digestive enzymes, in one embodiment, may comprise less than about 35%, 30%, 25%, 20%, 15%, or 10% of coated particles which can be sieved through #100 USSS mesh (150 μm). In some embodiments, the term “non-aerosolizable” refers to a digestive enzyme preparation where less than about 20% of the coated particles can be sieved through #100 USSS mesh (150 μm). In some embodiments, the term “non-aerosolizable” refers to a digestive enzyme preparation where less than about 15% of the coated particles can be sieved through #100 USSS mesh (150 μm). The digestive enzyme preparation can be an encapsulated digestive enzyme composite where the digestive enzyme particles can contain one, two, three, four, five, six, seven, eight, nine, ten, or more digestive enzymes.

Coated particles may be sieved to obtain coated particles of a suitable size or more uniform size range. The coated particles may be sieved through #40 USSS mesh and through USSS #140 USSS mesh. Coated particles that pass through the #40 USSS mesh but are retained by the #140 USSS mesh are of an appropriate size range. In some instances, the particles may also be screened to obtain particles of a suitable size for encapsulation by removing particles that are too fine or too large. For example, the coated particles may be sieved to obtain coated particles of a suitable size or more uniform size range for encapsulation. As a further example, the coated particles may be sieved through #40 USSS mesh and through USSS #140 USSS mesh. Coated particles that pass through the #40 USSS mesh but are retained by the #140 USSS mesh are of an appropriate size range for coating or encapsulation. Particles may also be screened by sieving through USSS #140, #120, #100, #80, #70, #60, #50, #45, or #40 USSS mesh, or any combination thereof.

Non-limiting examples of compositions that contain digestive enzymes have been discussed above. Other exemplary compositions are provided herein below. Each of the compositions described herein may be formulated with any combination of the described excipients.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a polymer coating.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. an enteric coating.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a Cellulose acetate phthalate (CAP).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a cellulose acetate trimellitate (CAT).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a hydroxyl propyl methyl cellulose phthalate (HPMCP).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a hydroxyl propyl methyl cellulose acetate succinate (HPMCAS).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a polyvinyl acetate phthalate (PVAP).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a methacrylic acid copolymer.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a shellac (Esters of Aleurtic Acid).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a Zein.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises an ethylcellulose (EC).

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a food grade lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a sorbitan monostearate, a sorbitan tristearate, or a calcium stearoyl lactylate.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a pharmaceutical grade lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a fully-hydrogenated soybean oil.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises one or more monoglycerides, one or more diglycerides, one or more triglycerides, fatty acids, esters of fatty acids, phospholipids, or a combination thereof.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises one or more monoglycerides, one or more diglycerides, one or more triglycerides, fatty acids, esters of fatty acids, phospholipids, or a combination thereof.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and a coating that comprises a soy lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a hydrogenated soy lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises the esters of fatty acids, wherein the esters of fatty acids are selected from the group consisting of acetic acid esters of mono- and diglycerides, citric acid esters of mono- and diglycerides, lactic acid esters of mono- and diglycerides, polyglycerol esters of fatty acids, propylene glycol esters of fatty acids, and diacetyl tartaric acid esters of mono- and diglycerides.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises a hydrogenated castor wax or a hydrogenated carnauba wax.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating that comprises an animal lipid or a vegetable lipid.

A pharmaceutical composition can comprise a. a core that comprises from about 10,000 to about 1,500,000 U.S.P. Units protease, from about 1,500 to about 282,000 U.S.P. Units lipase, and from about 1,000 to about 15,000,000 U.S.P. Units amylase; and b. a coating selected from the group consisting of a palm kernel oil, a soybean oil, a cottonseed oil, a canola oil, and a poultry fat.

A composition as described herein can comprise a. a core that comprises about 8,400 U.S.P. Units lipase, 35,000 U.S.P. Units protease, about 35,000 U.S.P. Units amylase, and b. a polymer coating. A composition as described herein can comprise a. a core that comprises about 134,400 U.S.P. Units lipase, about 560,000 U.S.P. Units protease, and about 560,000 U.S.P. Units amylase, and b. a polymer coating. A composition as described herein can comprise a. a core that comprises about 252,000 U.S.P. Units lipase, about 1,050,000 U.S.P. Units protease, and about 1,050,000 U.S.P. Units amylase, and b. a polymer coating.

A composition as described herein can comprise a. a core that comprises about 8,400 U.S.P. Units lipase, 35,000 U.S.P. Units protease, about 35,000 U.S.P. Units amylase, and b. an enteric coating. A composition as described herein can comprise a. a core that comprises about 134,400 U.S.P. Units lipase, about 560,000 U.S.P. Units protease, and about 560,000 U.S.P. Units amylase, and b. an enteric coating. A composition as described herein can comprise a. a core that comprises about 252,000 U.S.P. Units lipase, about 1,050,000 U.S.P. Units protease, and about 1,050,000 U.S.P. Units amylase, and b. an enteric coating.

A composition as described herein can comprise a. a core that comprises about 8,400 U.S.P. Units lipase, 35,000 U.S.P. Units protease, about 35,000 U.S.P. Units amylase, and b. a lipid coating. A composition as described herein comprises a. a core that comprises about 134,400 U.S.P. Units lipase, about 560,000 U.S.P. Units protease, and about 560,000 U.S.P. Units amylase, and b. a lipid coating. A composition as described herein that comprises a. a core can comprise about 252,000 U.S.P. Units lipase, about 1,050,000 U.S.P. Units protease, and about 1,050,000 U.S.P. Units amylase, and b. a lipid coating.

A composition as described herein can comprise a. a core that comprises about 8,400 U.S.P. Units lipase, 35,000 U.S.P. Units protease, about 35,000 U.S.P. Units amylase and b. a fully-hydrogenated soybean oil. A composition as described herein can comprise a. a core that comprises about 134,400 U.S.P. Units lipase, about 560,00 U.S.P. Units protease, and about 560,000 U.S.P. Units amylase, and b. a fully-hydrogenated soybean oil. A composition as described herein can comprise a. a core that comprises about 252,000 U.S.P. Units lipase, about 1,050,000 U.S.P. Units protease, and about 1,050,000 U.S.P. Units amylase, and b. a fully-hydrogenated soybean oil.

For explanation purposes only, in one non-limiting example, a subject is administered 8 capsules twice a day, wherein each capsule comprises digestive enzymes having about 560,000 U.S.P. Units protease and about 560,000 U.S.P. Units amylase. In an additional nonlimiting example, a subject is administered 10 capsules coated with an enteric coating stabile at a pH below about 7.2 to target delivery to the proximal ileum, wherein each capsule comprises about 252,000 U.S.P. Units lipase, about 1,050,000 U.S.P. Units protease, and about 1,050,000 U.S.P. Units amylase. In an additional nonlimiting example, a subject is administered 4 capsules four times a day, wherein the capsules have a dissolution time of approximately 90 minutes and comprise about 8,400 U.S.P. Units lipase, 35,000 U.S.P. Units protease, and about 35,000 U.S.P. Units amylase.

In one embodiment, a lipid-microencapsulated porcine pancreatic enzyme concentrate with high protease and low lipase levels is utilized for prophylaxis against infection from SARS-CoV-2. The non-pH dependent microencapsulation, applied by fluidized bed coating comprises pharmaceutical-grade fully hydrogenated soy oil (approximately 20% by weight) with a dissolution profile of approximately 85% in 60 minutes or less, allows the delivery of the digestive enzymes from a pharmaceutical composition in the duodenum. Drug delivery is sprinkles housed in a sachet that are sprinkled over food. Non-limiting exemplary dosing for subjects of various ages and exposure levels are given in Table 2.

In one embodiment, infant subjects with a weight greater than 4 Kg and less than three years old receive 2 doses per day, where each dose comprises about 90,000 U.S.P. Units protease, about 18,000 U.S.P. Units lipase, and about 108,000 U.S.P. Units of amylase delivered as lipid-microencapsulated porcine pancreatic enzyme concentrate sprinkles as a prophylaxis against SARS-CoV-2 infection. Each dose is approximately 450 mg of lipid-microencapsulated porcine pancreatic enzyme packaged in a tri-foil laminate sachet and given orally by sprinkling over food.

In one embodiment, child subjects three years of age and older receive 3 doses a day, where each dose comprises about 180,000 U.S.P. Units protease, about 36,000 U.S.P. Units lipase, and about 216,000 U.S.P. Units of amylase delivered as lipid-microencapsulated porcine pancreatic enzyme concentrate sprinkles as a prophylaxis against SARS-CoV-2 infection. Each dose is approximately 900 mg of lipid-microencapsulated porcine pancreatic enzyme packaged in a tri-foil laminate sachet and given orally by sprinkling over food.

In one embodiment, teen subjects receive 3 doses a day, where each dose comprises about 360,000 U.S.P. Units protease, about 72,000 U.S.P. Units lipase, and about 432,000 U.S.P. Units of amylase delivered as lipid-microencapsulated porcine pancreatic enzyme concentrate sprinkles as a prophylaxis against SARS-CoV-2 infection. Each dose is approximately 1,800 mg of lipid-microencapsulated porcine pancreatic enzyme packaged in a tri-foil laminate sachet and given orally by sprinkling over food.

In one embodiment, adults receive 3 doses a day, where each dose comprises about 720,000 U.S.P. Units protease, about 144,000 U.S.P. Units lipase, and about 864,000 U.S.P. Units of Amylase delivered as lipid-microencapsulated porcine pancreatic enzyme concentrate sprinkles as a prophylaxis against SARS-CoV-2 infection. Each dose is approximately 3,600 mg of lipid-microencapsulated porcine pancreatic enzyme packaged in a tri-foil laminate sachet and given orally by sprinkling over food.

In one embodiment, elderly subjects receive 3 doses a day, where each dose comprises about 360,000 U.S.P. Units protease, about 72,000 U.S.P. Units lipase, and about 432,000 U.S.P. Units of Amylase delivered as lipid-microencapsulated porcine pancreatic enzyme concentrate sprinkles as a prophylaxis against SARS-CoV-2 infection. Each dose is approximately 1,800 mg of lipid-microencapsulated porcine pancreatic enzyme packaged in a tri-foil laminate sachet and given orally by sprinkling over food.

In one embodiment, a lipid-microencapsulated porcine pancreatic enzyme concentrate with high protease and low lipase levels is utilized for treatment of COVID-19. The non-pH dependent microencapsulation allows the delivery of protease from a pharmaceutical composition in the early portion of the duodenum. Drug delivery is sprinkles housed in a sachet that are sprinkled over food. Dosing for subjects of various ages and exposure levels are given in Tables 3A and 3B.

In one embodiment, infant subjects with a weight greater than 4 Kg and less than three years old receive 2 doses per day, where each dose comprises about 90,000 U.S.P. Units protease, about 18,000 U.S.P. Units lipase, and about 108,000 U.S.P. Units amylase delivered as lipid-microencapsulated or lipid-coated digestive enzyme (e.g., porcine pancreatic digestive enzymes) concentrate sprinkles as treatment for COVID-19. Each dose is approximately 450 mg of lipid-microencapsulated or lipid-coated digestive enzymes (e.g., porcine pancreatic digestive enzymes) packaged in a tri-foil laminate sachet and given orally by sprinkling over food.

In one embodiment, child subjects three years of age and older receive 3 doses a day, where each dose comprises about 180,000 U.S.P. Units protease, about 36,000 U.S.P. Units lipase, and about 216,000 U.S.P. Units amylase delivered as lipid-microencapsulated or lipid-coated digestive enzyme (e.g., porcine pancreatic digestive enzymes) concentrate sprinkles as treatment for COVID-19. Each dose is approximately 900 mg of lipid-microencapsulated or lipid-coated digestive enzymes (e.g., porcine pancreatic digestive enzymes) packaged in a tri-foil laminate sachet and given orally by sprinkling over food.

In one embodiment, teen subjects receive 3 doses a day, where each dose comprises about 360,000 U.S.P. Units protease, about 72,000 U.S.P. Units lipase, and about 432,000 U.S.P. Units amylase delivered as lipid-microencapsulated or lipid-coated digestive enzyme (e.g., porcine pancreatic digestive enzymes) concentrate sprinkles as treatment for COVID-19. Each dose is approximately 1,800 mg of lipid-microencapsulated or lipid-coated digestive enzymes (e.g., porcine pancreatic digestive enzymes) packaged in a tri-foil laminate sachet and given orally by sprinkling over food.

In one embodiment, adults receive 3 doses a day, where each dose comprises about 720,000 U.S.P. Units protease, about 144,000 U.S.P. Units lipase, and about 864,000 U.S.P. Units amylase delivered as lipid-microencapsulated or lipid-coated digestive enzyme (e.g., porcine pancreatic digestive enzymes) concentrate sprinkles as treatment for COVID-19. Each dose is approximately 3,600 mg of lipid-microencapsulated or lipid-coated digestive enzymes (e.g., porcine pancreatic digestive enzymes) packaged in a tri-foil laminate sachet and given orally by sprinkling over food.

In one embodiment, elderly subjects receive 3 doses a day, where each dose comprises about 360,000 U.S.P. Units protease, about 72,000 U.S.P. Units lipase, and about 432,000 U.S.P. Units amylase delivered as lipid-microencapsulated or lipid-coated digestive enzyme (e.g., porcine pancreatic digestive enzymes) concentrate sprinkles as treatment for COVD-19. Each dose is approximately 1,800 mg of lipid-microencapsulated or lipid-coated digestive enzymes (e.g., porcine pancreatic digestive enzymes) packaged in a tri-foil laminate sachet and given orally by sprinkling over food.

In one embodiment, the dosing for prophylaxis by baseline exposure utilizing lipid-microencapsulated or lipid-coated digestive enzyme (e.g., porcine pancreatic digestive enzymes) concentrate (CoGen-2) is shown below in Table 2:

TABLE 2

| | Prophylaxis | | |
|---|---|---|---|
| | Subject Asymptomatic | Subject Asymptomatic | Subject Asymptomatic |
| | and has No SARS-CoV-2 | and has No SARS-CoV-2 | and has No SARS-CoV-2 |
| Baseline | Infection No | Infection Likely | Infection SARS-CoV-2 |
| Exposure | SARS-CoV-2 Exposure | SARS-CoV-2 Exposure | Definite Exposure |
| Infant | 2 doses per day. | 2 doses per day. | 2 doses per day. |
| (weight 4 kg | Each dose containing | Each dose containing | Each dose containing |
| or greater) | 90,000 U.S.P. Units | 90,000 U.S.P. Units | 90,000 U.S.P. Units |
| | protease, 18,000 U.S.P. | protease, 18,000 U.S.P. | protease, 18,000 U.S.P. |
| | Units lipase and 108,000 | Units lipase and 108,000 | Units lipase and 108,000 |
| | U.S.P. Units amylase. | U.S.P. Units amylase. | U.S.P. Units amylase. |
| Child | 3 doses per day. | 3 doses per day. | 3 doses per day. |
| (age 3 or | Each dose containing | Each dose containing | Each dose containing |
| greater) | 180,000 U.S.P. Units | 180,000 U.S.P. Units | 180,000 U.S.P. Units |
| | protease, 36,000 U.S.P. | protease, 36,000 U.S.P. | protease, 36,000 U.S.P. |
| | Units lipase and 216,000 | Units lipase and 216,000 | Units lipase and 216,000 |
| | U.S.P. Units amylase | U.S.P. Units amylase | U.S.P. Units amylase |
| Teen | 3 doses per day. | 3 doses per day. | 3 doses per day. |
| | Each dose containing | Each dose containing | Each dose containing |
| | 360,000 U.S.P. Uni | 360,000 U.S.P. Units | 360,000 U.S.P. Units |
| | Protease, 72,000 U.S.P. | protease, 72,000 U.S.P. | protease, 72,000 U.S.P. |
| | Units lipase and 432,000 | Units lipase and 432,000 | Units lipase and 432,000 |
| | U.S.P. Units amylase | U.S.P. Units amylase | U.S.P. Units amylase |
| Adult | 3 doses per day. | 3 doses per day. | 3 doses per day. |
| | Each dose containing | Each dose containing | Each dose containing |
| | 720,000 U.S.P. Units | 720,000 U.S.P. Units | 720,000 U.S.P. Units |
| | protease, 144,000 U.S.P. | protease, 144,000 U.S.P. | protease, 144,000 U.S.P. |
| | Units lipase and 864,000 | Units lipase and U.S.P. | Units lipase and 864,000 |
| | U.S.P. Units amylase | 864,000 Units Amylase | U.S.P. Units amylase |
| Elderly | 3 doses per day. | 3 doses per day. | 3 doses per day. |
| | Each dose containing | Each dose containing | Each dose containing |
| | 360,000 U.S.P. Units | 360,000 U.S.P. Units | 360,000 U.S.P. Units |
| | protease, 72,000 U.S.P. | protease, 72,000 U.S.P. | protease, 72,000 U.S.P. |
| | Units lipase and 432,000 | Units lipase and 432,000 | Units lipase and 432,000 |
| | U.S.P. Unit Amylase | U.S.P. Units amylase | U.S.P. Units amylase |

In another embodiment, the dosing for treatment of SARS-CoV-2 Infection (COVID-19) by severity utilizing lipid-microencapsulated porcine pancreatic enzyme concentrate (CoGen-2) is shown below in Tables 3A and 3B:

TABLE 3A

| | Treatment | | |
|---|---|---|---|
| Baseline | Subject Asymptomatic | Subject has | Subject has |
| Severity | Positive for COVID-19 | Mild COVID-19 | Moderate COVID-19 |
| Infant | 2 doses per day | 2 doses per day. | 2 doses per day. |
| (weight 4 kg | Each dose containing | Each dose containing | Each dose containing |
| or greater) | 90,000 U.S.P. Units | 90,000 U.S.P. Units | 90,000 U.S.P. Units |
| | protease, 18,000 U.S.P. | protease, 18,000 U.S.P. | protease, 18,000 U.S.P. |
| | Units lipase and 108,000 | Unit Lipase and 108,000 | Units lipase and 108,000 |
| | U.S.P. Units amylase. | U.S.P. Units amylase. | U.S.P. Units amylase. |
| Child | 3 doses per day. | 3 doses per day. | 3 doses per day. |
| (age 3 or | Each dose containing | Each dose containing | Each dose containing |
| greater) | 180,000 U.S.P. Units | 180,000 U.S.P. Units | 180,000 U.S.P. Units |
| | protease, 36,000 U.S.P. | protease, 36,000 U.S.P. | protease, 36,000 U.S.P. |
| | Units lipase and 216,000 | Units lipase and 216,000 | Units lipase and 216,000 |
| | U.S.P. Units amylase | U.S.P. Units amylase | U.S.P. Units amylase |
| Teen | 3 doses per day. | 3 doses per day. | 3 doses per day. |
| | Each dose containing | Each dose containing | Each dose containing |
| | 360,000 U.S.P. Units | 360,000 U.S.P. Units | 360,000 U.S.P. Units |
| | protease, 72,000 U.S.P. | protease, 72,000 U.S.P. | protease, 72,000 U.S.P. |
| | Units lipase and 432,000 | Units lipase and 432,000 | Units lipase and 432,000 |
| | U.S.P. Units amylase | U.S.P. Units amylase | U.S.P. Units amylase |
| Adult | 3 doses per day. | 3 doses per day. | 3 doses per day. |
| | Each dose containing | Each dose containing | Each dose containing |
| | 720,000 U.S.P. Units | 720,000 U.S.P. Units | 720,000 U.S.P. Units |
| | protease, 144,000 U.S.P. | protease, 144,000 U.S.P. | protease, 144,000 U.S.P. |
| | Units lipase and 864,000 | Units lipase and 864,000 | Units lipase and 864,000 |
| | U.S.P. Units amylase | U.S.P. Units amylase | U.S.P. Units amylase |
| Elderly | 3 doses per day. | 3 doses per day. | 3 doses per day. |
| | Each dose containing | Each dose containing | Each dose containing |
| | 360,000 U.S.P. Units | 360,000 U.S.P. Units | 360,000 U.S.P. Units |
| | protease, 72,000 U.S.P. | protease, 72,000 U.S.P. | protease, 72,000 U.S.P. |
| | Units lipase and 432,000 | Units lipase and 432,000 | Units lipase and 432,000 |
| | U.S.P. Units amylase | U.S.P. Units amylase | U.S.P. Units amylase |

TABLE 3B

| | Treatment | |
|---|---|---|
| Baseline | Subject has | Subject has |
| Severity | Severe COVID-19 | Critical COVID-19 |
| Infant | 2 doses per day. | 2 doses per day. |
| (weight 4 kg | Each dose containing | Each dose containing |
| or greater) | 90,000 U.S.P. Units | 90,000 U.S.P. Units |
| | protease, 18,000 U.S.P. | protease, 18,000 U.S.P. |
| | Units lipase and 108,000 | Units lipase and 108,000 |
| | U.S.P. Units amylase. | U.S.P. Units amylase. |
| Child | 3 doses per day. | 3 doses per day. |
| (age 3 or | Each dose containing | Each dose containing |
| greater) | 180,000 U.S.P. Units | 180,000 U.S.P. Units |
| | protease, 36,000 U.S.P. | protease, 36,000 U.S.P. |
| | Units lipase and 216,000 | Units lipase and 216,000 |
| | U.S.P. Units amylase | U.S.P. Units amylase |
| Teen | 3 doses per day. | 3 doses per day. |
| | Each dose containing | Each dose containing |
| | 360,000 U.S.P. Units | 360,000 U.S.P. Units |
| | protease, 72,000 U.S.P. | protease, 72,000 U.S.P. |
| | Units lipase and 432,000 | Units lipase and 432,000 |
| | U.S.P. Units amylase | U.S.P. Units amylase |
| Adult | 3 doses per day. | 3 doses per day. |
| | Each dose containing | Each dose containing |
| | 720,000 U.S.P. Units | 720,000 U.S.P. Units |
| | protease, 144,000 U.S.P. | protease, 144,000 U.S.P. |
| | Units lipase and 864,000 | Units lipase and 864,000 |
| | U.S.P. Units amylase | U.S.P. Units amylase |
| Elderly | 3 doses per day. | 3 doses per day. |
| | Each dose containing | Each dose containing |
| | 360,000 U.S.P. Units | 360,000 U.S.P. Units |
| | protease, 72,000 U.S.P. | protease, 72,000 U.S.P. |
| | Units lipase and 432,000 | Units lipase and U.S.P. |
| | U.S.P. Units amylase | 432,000 Units Amylase |

### SARS-CoV-2 Prophylaxis—Dosing Examples

An eight-month-old infant female subject weighing about 7.9 kg is asymptomatic for COVID-19 with definite SARS-CoV-2 exposure, but not diagnosed SARS-CoV-2 infection. As a prophylaxis against SARS-CoV-2 infection, the infant subject is orally administered 2 doses/day of lipid-microencapsulated or lipid-coated digestive enzyme (e.g., porcine pancreatic digestive enzymes) concentrate; each dose containing of about 90,000 U.S.P. Units (U) protease, about 18,000 U.S.P. U lipase, and about 108,000 U.S.P. U amylase. The non-pH dependent microencapsulation or coating applied by fluidized bed coating comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 6 weeks assuming no additional or ongoing SARS-CoV-2 exposure and no SARS-CoV-2 infection.

A five-year-old male subject is asymptomatic for COVID-19 with a likelihood of SARS-CoV-2 exposure and no diagnosed SARS-CoV-2 infection. As a prophylaxis against SARS-CoV-2 infection, the subject is orally administered 3 doses/day of lipid-microencapsulated or lipid-coated digestive enzyme (e.g., porcine pancreatic digestive enzymes) concentrate; each dose contains about 180,000 U.S.P. U protease, about 36,000 U.S.P. U lipase, and about 216,000 U.S.P. U amylase. The non-pH dependent microencapsulation or coating, applied by fluidized bed coating, comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 8 weeks assuming no additional or ongoing SARS-CoV-2 exposure and no SARS-CoV-2 infection.

A fourteen-year-old female subject is asymptomatic for COVID-19 with no SARS-CoV-2 exposure and no diagnosed SARS-CoV-2 infection. As a prophylaxis against SARS-CoV-2 infection, the subject is orally administered 3 doses/day of lipid-microencapsulated or lipid-coated digestive enzyme (e.g., porcine pancreatic digestive enzymes) concentrate; each dose containing about 360,000 U.S.P. U protease, about 72,000 U.S.P. U lipase, and about 432,000 U.S.P. U amylase. The non-pH dependent microencapsulation or coating, applied by fluidized bed coating, comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 16 weeks during the course of the COVID-19 pandemic, assuming no SARS-CoV-2 infection.

A thirty-five-year-old adult male subject is asymptomatic for COVID-19 with definite SARS-CoV-2 exposure and no diagnosed SARS-CoV-2 infection. As a prophylaxis against symptomatic COVID-19, the subject is orally administered 3 doses/day of lipid-microencapsulated or lipid-coated digestive enzyme (e.g., porcine pancreatic digestive enzymes) concentrate; each dose containing about 720,000 U.S.P. U protease, about 144,000 U.S.P. U lipase, and about 863, 000 U.S.P. U amylase. The non-pH dependent microencapsulation or coating, applied by fluidized bed coating, comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 8 weeks assuming no additional or ongoing SARS-CoV-2 exposure or SARS-CoV-2 infection.

A seventy-five-year-old elderly female subject is asymptomatic for COVID-19 with definite SARS-CoV-2 exposure and no diagnosed SARS-CoV-2 infection. As a prophylaxis against SARS-CoV-2 infection, the subject is orally administered 3 doses/day of lipid-microencapsulated or lipid-coated digestive enzyme (e.g., porcine pancreatic digestive enzymes) concentrate; each dose containing about 360,000 U.S.P. U protease, about 72,000 U.S.P. U lipase, and about 432,000 U.S.P. U amylase. The non-pH dependent microencapsulation or coating, applied by fluidized bed coating, comprises pharmaceutical-grade fully hydrogenated soy oil (approximately 20% by weight) with a dissolution profile of approximately 85% in 60 minutes or less. The drug delivery is sprinkles housed in a sachet that are sprinkled over food. The dosing is for about 8 weeks assuming no additional or ongoing SARS-CoV-2 exposure and no SARS-CoV-2 infection.

### COVID-19 Treatment—Dosing Examples

It will be understood that the following dosing examples are representative and non-limiting. A ten-month-old infant male subject weighing approximately 9.2 kg is asymptomatic for COVID-19 but does have a SARS-CoV-2 infection. As a treatment for the SARS-CoV-2 infection, the infant subject is orally administered 2 doses/day of lipid-microencapsulated or lipid-coated digestive enzyme (e.g., porcine pancreatic digestive enzymes) concentrate; each dose containing of about 90,000 U.S.P. Units (U) protease, 18,000 U.S.P. U lipase, and 108,000 U.S.P. U amylase. The non-pH dependent microencapsulation or coating (e.g., applied by fluidized bed coating) comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 8 weeks or until the subject is no longer infected with SARS-CoV-2.

An eight-year-old female subject has a mild COVID-19 caused by a SARS-CoV-2 infection and exhibits symptoms of fever, chills, cough, along with gastrointestinal symptoms of nausea and diarrhea. As a treatment for mild COVID-19, the subject is orally administered 3 doses/day of lipid-microencapsulated or lipid-coated digestive enzyme (e.g., porcine pancreatic digestive enzymes) concentrate; each dose contains about 180,000 U.S.P. Units (U) protease, about 36,000 U.S.P. U lipase, and about 216,000 U.S.P. U amylase. The non-pH dependent microencapsulation or coating comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 8 weeks or until the subject no longer has a symptom of COVID-19 or a SARS-CoV-2 infection.

A sixteen-year-old male has a moderate COVID-19 caused by a SARS-CoV-2 infection and exhibits the symptoms of fever, chills, cough along with shortness of breath with exertion. As a treatment for the moderate COVID-19, the subject is orally administered 3 doses/day of lipid-microencapsulated or lipid-coated digestive enzyme (e.g., porcine pancreatic digestive enzymes) concentrate; each dose containing about 360,000 U.S.P. U protease, about 72,000 U.S.P. U lipase, and about 432,000 U.S.P. U amylase. The non-pH dependent microencapsulation or coating comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 12 weeks or until the subject no longer has symptoms of COVID-19 or a SARS-CoV-2 infection.

A forty-year-old female subject has a severe COVID-19 caused by a SARS-CoV-2 infection and exhibits the symptoms of fever, chills, cough along with dyspnea loss of taste, loss of smell and shortness of breath at rest, respiratory distress, and a respiratory rate >30 per minute. As a treatment for severe COVID-19, the subject is orally administered 3 doses/day of lipid-microencapsulated or lipid-coated digestive enzyme (e.g., porcine pancreatic digestive enzymes) concentrate, each dose containing about 720,000 U.S.P. U protease, about 144,000 U.S.P. U lipase, and about 863, 000 U.S.P. U amylase. The non-pH dependent microencapsulation or coating comprises pharmaceutical-grade fully hydrogenated soy oil (approximately 20% by weight) with a dissolution profile of approximately 85% in 60 minutes or less. The drug delivery is sprinkles housed in a sachet that are sprinkled over food. The dosing is for about 12 weeks or until the subject no longer has symptoms of COVID-19 or a SARS-CoV-2 infection.

An eighty-year-old male subject has a critical COVID-19 caused by a SARS-CoV-2 infection and exhibits the symptoms of fever, chills, dyspnea, along with a clinical diagnosis of respiratory failure. As a treatment for the critical SARS-CoV-2 infection, the subject is orally administered 3 doses/day of lipid-microencapsulated or lipid-coated digestive enzyme (e.g., porcine pancreatic digestive enzymes) concentrate; each dose containing about 360,000 U.S.P. U protease, about 72,000 U.S.P. U lipase, and about 432,000 U.S.P. U amylase. The non-pH dependent microencapsulation or coating comprises pharmaceutical-grade, fully hydrogenated soy oil (approximately 20% by weight) with a dissolution profile of approximately 85% in 60 minutes or less. The drug delivery is sprinkles housed in a sachet that are sprinkled over food. The dosing is for about 16 weeks or until the subject no longer has a SARS-CoV-2 infection.

### Combination Therapy

It will be understood that administration of a pharmaceutical composition described herein can be supplemented by one or more additional therapies or drugs such as, for example, respiratory therapy; one or more blood thinners or anti-coagulants; statins, intubation; hydroxy chloroquine; one or more antibiotics (e.g., doxycycline, Azithromycin, etc.); one or more decongestants (e.g., MUCINEX®, SUDAFED®, etc.); one or more anti-histamines and/or glucocorticoids (e.g., ZYRTEC®, CLARITIN®, ALLEGRA®, fluticasone luroate, etc.); one or more pain relievers (e.g., acetaminophen); one or more zinc-containing medications (e.g., ZYRTEC®, etc.); Azithromycin, hydroquinolone, or a combination thereof; one or more integrase inhibitors (e.g., Bictegravir, dolutegravir (TIVICAY®), elvitegravir, raltegravir, or a combination thereof); one or more nucleoside/nucleotide reverse transcriptase inhibitors (NRTIs; e.g., abacavir (ZIAGEN®), emtricitabine (EMTRIVA®), lamivudine (EPIVIR®), tenofovir alafenamide fumarate (VEMLIDY®), tenofovir disoproxil fumarate (VIREAD®), zidovudine (Retrovir®), didanosine (VIDEX®, VIDEX EC®), stavudine (ZERIT®), or a combination thereof); a combination of NRTIs (e.g., (i) abacavir, lamivudine, and zidovudine (TRIZIVIR®); (ii) abacavir and lamivudine (EPZICOM®); (iii) emtricitabine and tenofovir alafenamide fumarate (DESCOVY®); (iv) emtricitabine and tenofovir disoproxil fumarate (TRUVADA®); (v) lamivudine and tenofovir disoproxil fumarate (CIMDUO®, TEMIXYS®); (vi) lamivudine and zidovudine (COMBIVIR®); etc.); a combination of DESCOVY® and TRUVADA®; one or more non-nucleoside reverse transcriptase inhibitors (NNRTIs; e.g., doravirine (PIFELTRO®), efavirenz (SUSTIVA®), etravirine (INTELENCE®), nevirapine (VIRAMUNE®, VIRAMUNE XR®), rilpivirine (EDURANT®), delavirdine (RESCRIPTOR®), or a combination thereof); one or more Cytochrome P4503A (CYP3A) inhibitors (e.g., cobicistat (TYBOST®), ritonavir (NORVIR®), etc.); one or more protease inhibitors (PIs; e.g., atazanavir (REYATAZ®), darunavir (PREZISTA®), fosamprenavir (LEXIVA®), lopinavir, ritonavir (NORVIR®), tipranavir (APTIVUS®), etc.); one or PIs in combination with cobicistat, ritonavir, Lopinavir, Tipranavir, Atazanavir, fosamprenavir, indinavir (CRIXIVAN®), nelfmavir (VIRACEPT®), saquinavir (INVIRASE®), or a combination thereof; Atazanavir; fosamprenavir; a combination of Atazanavir, darunavir and cobicistat; one or more fusion inhibitors (e.g., enfuvirtide (FUZEON®); one or more post-attachment inhibitors (e.g., ibalizumab-uiyk (TROGARZO®)); one or more Chemokine coreceptor antagonists (CCR5 antagonists; e.g., maraviroc (SELZENTRY®)); and one or more viral entry inhibitors (e.g., enfuvirtide (FUZEON®), ibalizumab-uiyk (TROGARZO®), maraviroc (SELZENTRY®), etc.); or a combination thereof.

Non-limiting examples of combinations include pharmaceutical compositions described herein to be administered with one or more of the following: (1) Azithromycin, hydroquinolone, or a combination thereof, (2) darunavir and cobicistat (PREZCOBIX®), (3) lopinavir and ritonavir (KALETRA®), (4) abacavir, lamivudine, and zidovudine (TRIZIVIR®), (5) abacavir and lamivudine (EPZICOM®), (6) emtricitabine and tenofovir alafenamide fumarate (DESCOVY®), (7) emtricitabine and tenofovir disoproxil fumarate (TRUVADA®), (8) lamivudine and tenofovir disoproxil fumarate (CIMDUO®, TEMIXYS®), (9) lamivudine and zidovudine (Combivir), (10), atazanavir and cobicistat (EVOTAZ®), (11) doravirine, lamivudine, and tenofovir disoproxil fumarate (DELSTRIGO®), (12) efavirenz, lamivudine, and tenofovir disoproxil fumarate (SYMFI®), (13) efavirenz, lamivudine, and tenofovir disoproxil fumarate (SYMFI LO®), (14) efavirenz, emtricitabine, and tenofovir disoproxil fumarate (ATRIPLA®), (15) emtricitabine, rilpivirine, and tenofovir alafenamide fumarate (ODEFSEY®), (16) emtricitabine, rilpivirine, and tenofovir disoproxil fumarate (COMPLERA®), (17) elvitegravir, cobicistat, emtricitabine, and tenofovir disoproxil fumarate (STRIBILD®), (18) elvitegravir, cobicistat, emtricitabine, and tenofovir alafenamide fumarate (GENVOYA®), (19) abacavir, dolutegravir, and lamivudine (TRIUMEQ®), (20) bictegravir, emtricitabine, and tenofovir alafenamide fumarate (BIKTARVY®), (21) dolutegravir and lamivudine (DOVATO®), (22) dolutegravir and rilpivirine (JULUCA®), (23) darunavir, cobicistat, emtricitabine, and tenofovir alafenamide fumarate (SYMTUZA®).

Non-limiting examples of combinations include pharmaceutical compositions described herein to be administered with one or more blood thinners. Blood thinners to be co-administered include, but are not limited to, anti-platelet, and anti-coagulation medications. Antiplatelet medications are those such as, for example, aspirin, clopidogrel (PLAVIX®); prasugrel (EFFIENT®); ticlopidine (TICLID®); ticagrelor (BRILINTA®); and combinations thereof. Anticoagulants include, but are not limited to, Warfarin (COUMADIN®, JANTOVEN®); Heparin (e.g., FRAGMIN®, INNOHEP®, and LOVENOX®); Eabigatran (PRADAXA®); Epixaban (ELIQUIS®); Non-vitamin K antagonist oral anticoagulants (NOACs) such as, for example, Rivaroxaban (XARELTO®); Factor Xa inhibitors such as, for example, Edoxaban (SAVAYSA®), Fondaparinux (ARIXTRA®); and combinations thereof.

Other non-limiting examples of combinations include pharmaceutical compositions described herein to be administered with one or more gastrointestinal modulators (e.g., an agent that reduces stomach acid in a subject). A gastrointestinal modulators of stomach acid can be utilized in combination with coated or uncoated digestive enzyme compositions are utilized to prophylaxis against or treat coronavirus infections. The combination of gastrointestinal modulators of stomach acid (also referred to as agents that reduce stomach acid) and pharmaceutical compositions enhance the controlled targeted delivery of enzymes having increased stability and enhanced administration properties.

As previously discussed, coronavirus receptors lie in various portions of the GI tract and in various organs, dependent upon the type of coronavirus. Delivery to the appropriate receptor sites may be more precise in the gut with more efficient delivery through the combination of appropriate coatings and GI modulators.

The use of gastrointestinal modulators in conjunction with one or more coatings for the digestive enzymes can provide for many different dissolution profiles to achieve optimal efficacy and delivery of the digestive enzymes. In another example of the present disclosure the amount and or types of gastrointestinal modulators are varied around a fixed enzyme coating to maximize efficacious release of digestive enzymes in the appropriate portion(s) of the gastrointestinal tract to treat the symptoms or causes of one or more diseases.

In another example, the amount and/or types of gastrointestinal modulators are fixed and the thickness of the coating is varied to maximize efficacious release of digestive enzymes in the appropriate portion(s) of the gastrointestinal tract to treat the symptoms or causes of one or more diseases.

In another example, the timing of giving one or more gastrointestinal modulators are varied and the thickness of the coating is fixed to maximize efficacious release of digestive enzymes in the appropriate portion(s) of the gastrointestinal tract to prophylaxis or treat an infection or symptoms thereof from one or more coronaviruses.

In one aspect, coated or uncoated compositions comprising one or more digestive enzyme(s) are utilized in combination with Histamine-2 receptor antagonists (H2-blockers) including, but not limited to, ranitidine (tradename ZANTAC®), nizatidine (tradename AXID®) famotidine (tradename PEPCID®), cimetidine (tradename TAGAMET®) to enhance the controlled and targeted delivery of one or more digestive enzyme(s) to patients.

In one aspect, coated or uncoated compositions comprising one or more digestive enzyme(s) are utilized in combination with Proton Pump Inhibitors (PPIs) including, but not limited to omeprazole (tradename PRILOSEC®) esomeprazole (tradename NEXIUM®) omeprazole and sodium bicarbonate (tradename ZEGERID®) lansoprazole (tradename PREVACID®) dexlansoprazole (tradename DEXILANT®) rabeprazole (tradename ACIPHEX®) pantoprazole (tradename PROTONIX®) to enhance the controlled and targeted delivery of one or more digestive enzyme(s) to patients.

In one aspect, coated or uncoated compositions comprising one or more digestive enzyme(s) are utilized in combination with Mucosal Protectants including, but not limited to, sucralfate (tradename CARAFATE®) bismuth subsalicylate (tradename PEPTO-BISMOL®) to enhance the controlled and targeted delivery of one or more digestive enzyme(s) to patients.

In one aspect, coated or uncoated compositions comprising one or more digestive enzyme(s) are utilized in combination with Pro-kinetic Agents including, but not limited to, metoclopramide (tradename Reglan) bethanecol (tradename URECHOLINE®) to enhance the controlled and targeted delivery of one or more digestive enzyme(s) to patients.

In one aspect, coated or uncoated compositions comprising one or more digestive enzyme(s) are utilized in combination with Anticholinergic Agents including, but not limited to, scopolamine (tradename TransdermScop), trihexyphenidyl (tradename Artane), benztropine (tradename COGENTIN®), dicyclomine (tradename BENTYL®), glycopyrrolate (tradename ROBINUL®), hyoscyamine (tradename LEVSIN®), or atropine to enhance the controlled and targeted delivery of one or more digestive enzyme(s) to patients.

### Gastric Acid Suppression

Therapies to limit the ability of stomach acid to digest substrate do so either by limiting the amount of stomach acid or by limiting its contact with substrate. When digestive enzymes are administered orally, the digestive enzymes are exposed to highly acidic conditions in the stomach, with a pH of 1 or 2, as well as gastric proteases which denature and degrade the enzymes.

Gastric acid is a secretion produced in the stomach. It is one of the main isotonic solutions secreted, together with several enzymes and intrinsic factors. Chemically it is an acid solution with a pH of 1 to 2 in the stomach lumen, consisting mainly of hydrochloric acid (HCl) (around 0.5%, or about 5000 parts per million), and large quantities of potassium chloride (KCl) and sodium chloride (NaCl).

Gastric acid is produced by parietal cells (also called oxyntic cells) in the stomach. Its secretion is a complex and relatively energetically expensive process. Parietal cells contain an extensive secretory network (called canaliculi) from which the gastric acid is secreted into the lumen of the stomach. These cells are part of epithelial fundic glands in the gastric mucosa. The pH of gastric acid is 2 to 3 in the human stomach lumen, the acidity being maintained by the proton pump H+/K+ ATPase (also referred to as the hydrogen ion pump herein). The parietal cell releases bicarbonate into the blood stream in the process, which causes the temporary rise of pH in the blood, known as alkaline tide.

The resulting highly acidic environment in the stomach lumen causes proteins from food to lose their characteristic folded structure (or denature). This exposes the protein's peptide bonds. The chief cells of the stomach secrete enzymes for protein breakdown (inactive pepsinogen and renin). Gastric acid activates pepsinogen into pepsin—this enzyme then helps digestion by breaking the bonds linking amino acids, a process known as proteolysis.

Gastric acid secretion happens in several steps. Chloride and hydrogen ions are secreted separately from the cytoplasm of parietal cells and mixed in the canaliculi. Gastric acid is then secreted into the lumen of the oxyntic gland and gradually reaches the main stomach lumen. Chloride and sodium ions are secreted actively from the cytoplasm of the parietal cell into the lumen of the canaliculus. This creates a negative potential of −40 mV to −70 mV across the parietal cell membrane that causes potassium ions and a small number of sodium ions to diffuse from the cytoplasm into the parietal cell canaliculi.

The enzyme carbonic anhydrase catalyzes the reaction between carbon dioxide and water to form carbonic acid. This acid immediately dissociates into hydrogen and bicarbonate ions. The hydrogen ions leave the cell through H+/K+ ATPase antiporter pumps. At the same time sodium ions are actively reabsorbed. This means the majority of secreted K+ and Na+ ions return to the cytoplasm. In the canaliculus, secreted hydrogen and chloride ions mix and are secreted into the lumen of the oxyntic gland.

The highest concentration that gastric acid reaches in the stomach is 160 mM in the canaliculi. This is about 3 million times that of arterial blood, but almost exactly isotonic with other bodily fluids. The lowest pH of the secreted acid is 0.8, but the acid is diluted in the stomach lumen to a pH between 1 and 3.

There are three phases in the secretion of gastric acid: 1. the cephalic phase: 30% of the total gastric acid to be produced is stimulated by anticipation of eating and the smell or taste of food; 2. the gastric phase: 60% of the acid secreted is stimulated by the distention of the stomach with food and digestion produces proteins, which causes even more gastrin production; and 3. the intestinal phase: the remaining 10% of acid is secreted when chyme enters the small intestine, and is stimulated by small intestine distention.

Gastric acid production is regulated by both the autonomic nervous system and several hormones. The parasympathetic nervous system, via the vagus nerve, and the hormone gastrin stimulate the parietal cell to produce gastric acid, both directly acting on parietal cells and indirectly, through the stimulation of the secretion of the hormone histamine from enterochromaffin-like cells (ECL). Vasoactive intestinal peptide, cholecystokinin, and secretin all inhibit production.

The production of gastric acid in the stomach is tightly regulated by positive regulators and negative feedback mechanisms. Four types of cells are involved in this process: parietal cells, G cells, D cells and enterochromaffine-like cells. Besides this, the endings of the vagus nerve (X) and the intramural nervous plexus in the digestive tract influence the secretion significantly.

In one example, the release of digestive enzymes is timed to release specific percentages of enzymes in specific portions of the gastrointestinal tract by a combined use of gastrointestinal modulators and coating technologies.

In another example, the amount and or types of gastrointestinal modulators are varied around a fixed enzyme coating to maximize efficacious release of digestive enzymes in the appropriate portion(s) of the gastrointestinal tract to treat the symptoms or causes of coronavirus infections.

In another example, the amount and or types of gastrointestinal modulators are fixed and the thickness of the enzyme coating is varied to maximize efficacious release of digestive enzymes in the appropriate portion(s) of the gastrointestinal tract to treat the symptoms or causes of coronavirus infections.

In another example, the timing of giving one or more gastrointestinal modulator(s) are varied and the thickness of the coating is fixed to maximize efficacious release of digestive enzymes in the appropriate portion(s) of the gastrointestinal tract to prophylaxis or treat an infection or symptoms thereof from one or more coronavirus infections.

In another example, the coatings and enzymes are concentrically nested to allow timed release of enzymes in more than one portion of the gastrointestinal tract to prophylaxis or treat an infection or the symptoms thereof treat the symptoms thereof for one or more coronavirus(es).

Nerve endings in the stomach secrete two stimulatory neurotransmitters: acetylcholine and gastrin-releasing peptide. Their action is both direct on parietal cells and mediated through the secretion of gastrin from G cells and histamine from enterochromaffine-like cells. Gastrin acts on parietal cells directly and indirectly too, by stimulating the release of histamine. The release of histamine is the most important positive regulation mechanism of the secretion of gastric acid in the stomach. Its release is stimulated by gastrin and acetylcholine and inhibited by somatostatin.

Parietal cells in the stomach produce acid necessary for digestion. Histamine release stimulates these cells to do so. Specific receptors on the parietal cell membrane, designated histamine-2 receptors, react to the presence of histamine at their active sites by beginning production of acid. Hydrogen ions, the essential building blocks of stomach acid, are then pumped into the stomach. Histamine-2 receptor antagonists (H2-blockers) are a class of drugs used to decrease the amount of acid produced in the stomach by inhibiting the ability of histamine to stimulate the histamine receptor on parietal cells. The acid-suppressing effects of most H2-blockers range from 6 to 24 hours. Examples of H2-blockers include ZANTAC® (ranitidine), ARID® (nizatidine), PEPCID® (famotidine) and TAGAMET® (cimetidine).

Histamine 2-receptor blockers (H2-RBs) also come in various dosage forms. All come in a tablet form and ranitidine also comes in a soluble tablet as well as a syrup. Famotidine is available in a chewable tablet and a suspension.

Proton pump inhibitors (PPIs) are a class of drugs used to decrease the amount of acid produced in the stomach by irreversibly inhibiting the hydrogen ion pump from working. The acid-suppressing effects of most PPIs can last as long as 24 hours. Examples of PPIs include PRILOSEC® (omeprazole), NEXIUM® (esomeprazole), ZEGERID (omeprazole+ sodium bicarbonate), PREVACID® (lansoprazole), DEXILANT® (dexlansoprazole), ACIPHEX® (rabeprazole), and PROTONIX® (pantoprazole).

Proton-pump inhibitors (PPIs) come in various dosage forms. Examples of PPIs that come in a tablet form are pantoprazole, rabeprazole and omeprazole. Omeprazole, esomeprazole, lansoprazole and dexlansoprazole all come in capsule form and can either be swallowed whole or opened and their contents sprinkled over select foods or in select juices as noted by their respective manufacturers. Lansoprazole is also available as a soluble tablet. Esomeprazole is available in a granule formulation. Omeprazole also comes in a combination with NaHCO in capsule and granule formulations.

Mucosal protectants provide a barrier that stomach acid must first penetrate before contacting digestive material and include sucralfate, bismuth subsalicylate, ranitidine bismuth citrate (RBC), and bismuth subgallate. Sucralfate is a mucosal protectant that attenuates the power of stomach acid in digesting substrate by coating the GI tract and providing a barrier that acid must first penetrate before contacting digestible material. Sucralfate (CARAFATE®) is a complex metal salt of sulfated sucrose. Although the sucralfate molecule contains aluminum hydroxide, the agent has little acid-neutralizing capacity. It is an aluminum salt of sulfated sucrose that disassociates under the acidic conditions in the stomach. It is speculated that the sucrose polymerizes and binds to protein in the ulcer crater to produce a kind of protective coating that can last for up to 6 hours. When exposed to gastric acid, the aluminum hydroxide dissociates, leaving sulfate anions that can bind electrostatically where it appears to form a protective barrier that may prevent further acid-peptic attack. Because of the lack of systemic absorption, sucralfate appears to have no systemic toxicity. Similar agents include PEPTO-BISMOL® (bismuth subsalicylate), ranitidine bismuth citrate (RBC) and bismuth subgallate. Bismuth preparations have been used widely to treat diarrhea, abdominal pain, and dyspepsia for hundreds of years. Two colloidal preparations of bismuth that have been most commonly used are colloidal bismuth subcitrate and bismuth subsalicylate (e.g., PEPTO-BISMOL®). The bismuth forms complexes with mucus to form a protective barrier that may prevent further acid-peptic attack. Bismuth is largely unabsorbed and is excreted in the feces.

Pro-kinetic agents are typically used to increase lower esophageal sphincter (LES) pressure and also accelerate gastric emptying by stimulating the smooth muscles of the GI tract. Examples include PROPULSID® (cisapride), REGLAN® (metoclopramide), and URECHOLINE® (bethanecol).

The combination of gastrointestinal modulator agents in a pharmaceutical composition described herein in appropriate therapeutic dosages would be beneficial for enhancing their efficacy. Concerning agents that decrease the amount of acid present to digest substrate, more unaffected compositions comprising one or more digestive enzyme(s) (coated or otherwise) would be able to make it to its target site in the small intestine. Concerning agents that limit the amount of time acid contacts substrate, compositions comprising one or more digestive enzyme(s) (coated or otherwise) would arrive at its target site in the small intestine sooner and with less degradation.

While certain embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the methods and structures within the scope of the following claims and their equivalents be covered thereby.

### Certain Definitions

All terms are intended to be understood as they would be understood by a person skilled in the art and. Unless defined otherwise, all technical and scientific terms used herein have the same meaning as commonly understood by one of ordinary skill in the art to which the disclosure pertains. Accordingly, the terminology used herein is for the purpose of describing particular embodiments only and is not intended to be limiting. Although any methods and materials similar or equivalent to those described herein can be used in the practice for testing of the present disclosure, the preferred materials and methods are described herein.

Reference in the specification to “certain instances,” “some instances,” “an instance,” “one instance,” “other instances,” “some embodiments,” “an embodiment,” “one embodiment” or “other embodiments” means that a particular feature, structure, or characteristic described in connection with the embodiments is included in at least some instances/embodiments, but not necessarily all instances/embodiments, of the present disclosure. To facilitate an understanding of the present disclosure, a number of terms and phrases are defined below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” as used herein, generally refers to a range that is 1%, 2%, 5%, 10%, 15% greater than or less than (±) a stated numerical value within the context of the particular usage. For example, “about 10” would include a range from 8.5 to 11.5. As used herein, the terms “about” and “approximately,” when used to modify a numeric value or numeric range, indicate that deviations of up to about 0.2%, about 0.5%, about 1%, about 2%, about 5%, about 7.5%, or about 10% (or any integer between about 1% and 10%) above or below the value or range remain within the intended meaning of the recited value or range.

As used in this specification and the appended claims, the singular forms “a,” “an,” and “the” include plural references unless the context clearly dictates otherwise. Thus, for example, references to “a method” include one or more methods, and/or steps of the type described herein and/or which will become apparent to those persons skilled in the art upon reading this disclosure.

Polypeptides (e.g., proteins) described herein can be isolated and/or purified from their natural environment in substantially pure or homogeneous form.

There are two types of pancreatic enzymes which have United States Pharmacopeia (U.S.P.) designations: pancreatin and pancrealipase.

“Pancreatin” is a substance containing enzymes, principally amylase, lipase, and protease, obtained from the pancreas of the hog Sus scrofa Linne var. domesticus Gray (Fam. Suidae) or of the ox Bos Taurus Linne (Fam. Bocidae). Pancreatin contains, in each mg, not less than 25 U.S.P. Units of amylase activity, not less than 2 U.S.P. Units of lipase activity, and not less than 25 U.S.P. of protease activity. Pancreatin of a higher digestive power may be labeled as a whole-number multiple of the three minimum activities or may be diluted by admixture with lactose, or with sucrose containing not more than 3.25 percent of starch, or with pancreatin of a lower digestive power. Pancreatin can be provided as a crystalline substance.

In contrast, “pancrealipase” refers to a cream-colored, amorphous powder, having a faint, characteristic meaty odor, which contains lipase in an amount of not less than 24 U.S.P. Units/mg; protease in an amount of not less than 100 U.S.P. Units/mg; and amylase in an amount of not less than 100 U.S.P. Units/mg; with not more than 5% fat and not more than 5% loss on drying.

“CREON®” is a form of pancrealipase that is sold as formulations of (i) 3,000 Units of a lipase, 9,500 Units of a protease, 15,000 Units of an amylase; (ii) 6,000 Units of a lipase, 19,000 Units of a protease, 30,000 Units of an amylase; (iii) 12,000 Units of a lipase, 38,000 Units of a protease, 60,000 Units of an amylase; (iv) 24,000 Units of a lipase, 76,000 Units of a protease, and 120,000 Units of an amylase; or (v) 36,000 Units of a lipase, 114,000 Units of a protease, and 180,000 Units of an amylase. CREON® formulations are known to be irritating to mucosa of a subject and also is known to cause the following adverse side effects: Abdominal pain, abnormal feces, cough, dizziness, flatulence, headache, weight decreased; hyperuricemia, fibrosing colonopathy (with high doses), and/or allergic reactions.

As used herein, the term “non-aerosolizable” will be used to refer to coated particles where substantially all of the coated particles are large enough to eliminate or reduce aerosolization upon pouring compared to uncoated digestive enzyme.

The phrase “pharmaceutically acceptable” refers to molecular entities and compositions that are physiologically tolerable.

An enzyme described herein can be isolated, purified, recombinant, synthetic, or a combination thereof. For example, an enzyme can be produced recombinantly or synthetically. In some instances, an enzyme can be produced recombinantly or synthetically and then isolated and/or purified.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

The following experiments tested and verified the cytoprotective effects of both Pancreatic Enzyme Concentrate and Microencapsulated Pancreatic Enzyme Concentrate in various concentrations against cell death and SARS-CoV-2 infection spread.

### Materials and Methods

### Cell Preparation:

The cell line utilized for the infection and plaque assays is Vero E6 cells (ATCC® CRL-1586). These cells were grown from a frozen aliquot of a laboratory working cell line. Passage number is limited to no more than 50 passages from the original aliquot. Cells were grown in T150 flasks in 1×DMEM (ThermoFisher cat. no. 12500062) supplemented with 2 mM L-glutamine (Hyclone cat. no. H30034.01), non-essential amino acids (Hyclone cat. no. SH30238.01), and 10% heat inactivated Fetal Bovine Serum (FBS) (Atlas Biologicals cat. no. EF-0500-A).

On the day prior to executing the assay, Vero E6 cells were removed from T150 flasks by trypsinization (0.25% Trypsin, Coming cat. no. 25-053-C1) and measured for count and viability by Hemocytometer in trypan blue. Cells were resuspended to 2.7×10⁵ cells per mL in 1×DMEM (supplemented as indicated above) and plated at 0.15 mL per well (40,000 cells/well) in 96-well plates. The plates were then incubated for approximately 24 hours to allow cell adherence at 37° C., 5% CO₂.

### Virus Stocks:

The virus strain used for the assay was SARS-CoV2, USA WA 01/2020, CSU V2 03/17/202 passage 3. Virus stocks were obtained from BEI Resources and amplified in Vero E6 cells to Passage 3 (P3) with a titer of 1.6×10⁶ PFU/mL. Stocks were stored at −80° C. Virus infection of indicated wells were carried out with two MOI (0.001 and 0.0001). See, plate format of Table 19.

Compounds, Source, and Concentration

| | | |
|---|---|---|
| Pancreatic Enzyme | Lot 2226-0001 | 3 mg/ml in PBS* |
| Concentrate (100 g) | | |
| Pancreatic Enzyme | Lot 2226-0003 | 3 mg/ml in PBS |
| Concentrate (100 g) | | |
| Pancreatic Enzyme | Lot 2226-0004 | 3 mg/ml in PBS |
| Concentrate (100 g) | | |
| Microencapsulated | Curemark Supplied | 3 mg/ml in PBS |
| Pancreatic Enzyme | | |
| Concentrate | | |

*PBS: phosphate buffered saline.

### Assay Setup:

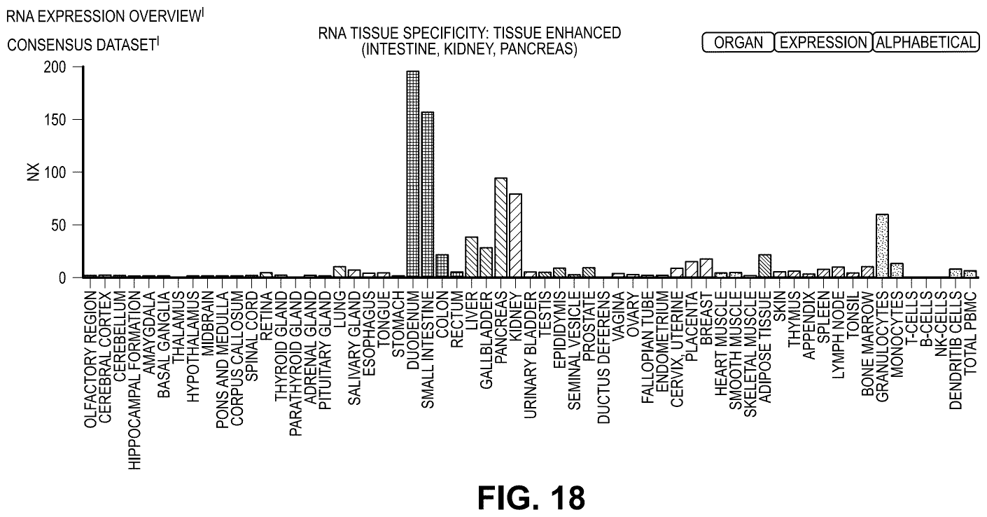

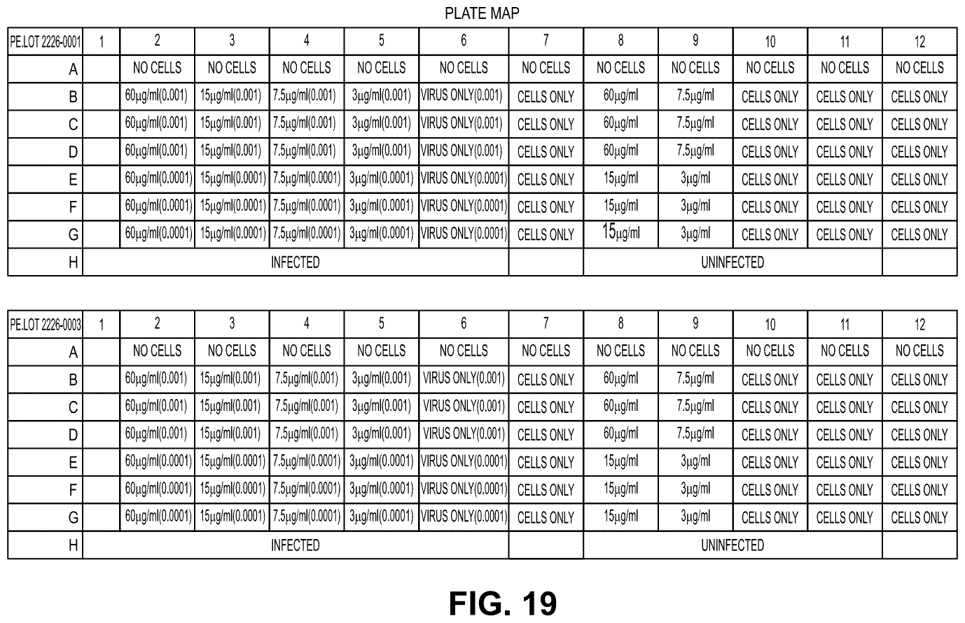

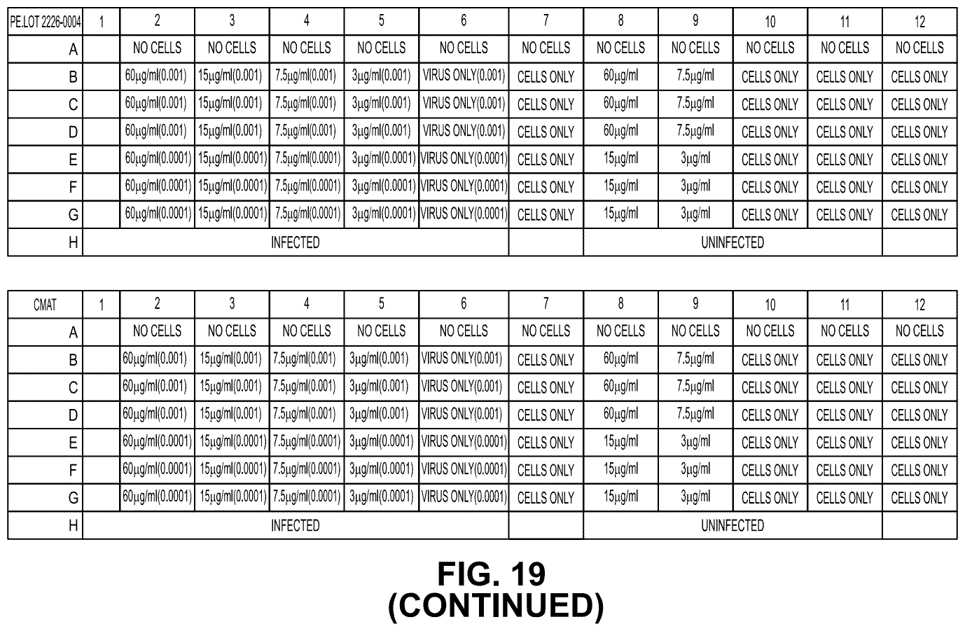

1. Stock solutions were prepared in PBS (PH 7.2), then incubated at 37° C. for 30 min, and vortexed regularly.
2. Stock solutions were centrifugated at 4° C. at 1400 rpm for 10 minutes.
3. Stock solutions were diluted (1:50, 1:200, 1:400, or 1:1000) in 1×DMEM+++ media.
4. Four 96-well plates were set up as shown in FIG. 19, Plate Map.
3. Pre-treatment: Media was aspirated from the 96 wells, 100 μL of the compound dilutions (and controls) as shown above (step 2) were added, and the plate(s) incubated at 37° C./5% CO₂ for 1 hour (hr).
4. After the pre-treatment, virus (MOI 0.001 and 0.0001) was added dropwise into the media, plates were sealed with aeroseal and incubated for 72 hr at 37° C./5% CO₂. For cytotoxicity assays, no virus was added.
5. At 72 hr, supernatants were harvested and stored at −80° C. Cytoprotection was measured using neutral red.
5a. Neutral red (NR) solution (0.33% NRS, Sigma Aldrich cat. no. N2889) maintained at room temperature was diluted 1:24 in 10% 1×DMEM warmed to 37° C. Immediately after dilution the solution was centrifuged at 4000 rpm in a tabletop centrifuge for 30 min (to remove any NR crystals).
5b. The diluted NR solution was added to the cells (150 μl/well).
5c. The plate was incubated at 37° C., 5% CO₂ for approximately 2 hours.
5d. NR solution was removed, and NR solubilization solution (1% glacial acetic acid in 50% ethanol) was added to each well at 150 μl/well. Plates were incubated at room temperature for 10 minutes, and solubilization solution mixed by pipetting.
5e. 130 μL from each well was transferred to a new plate.
5f. Absorbance was read on a plate reader at 540 n. Plate background absorbance was read at 590 nm.

### Plaque Assay:

Four 10-fold serial dilutions of the supernatants were carried out for each sample in 1×DMEM. 200 μL of these dilutions were dispensed onto wells in a 12-well plate. Plates were incubated at 37° C./5% CO₂ for 1 hour to allow virus to adsorb (plates were rocked intermittently). After an hour, wells were overlaid with 2% agarose and 2×DMEM (supplemented with 10% FBS). Plaque assays were incubated at 37° C./5% CO₂ for 72 hr. Cell Staining: 72 hrs. post-plaque assay, cells were stained with 1×PBS and neutral red solution (NRS) (0.33% NRS, Sigma Aldrich cat. no. N2889) at a ratio of 11.5 mL 1×PBS and 0.5 mL NRS per 12-well plate (1 ml/well). Staining was carried out overnight (about 12 hrs).

### Experiment 1

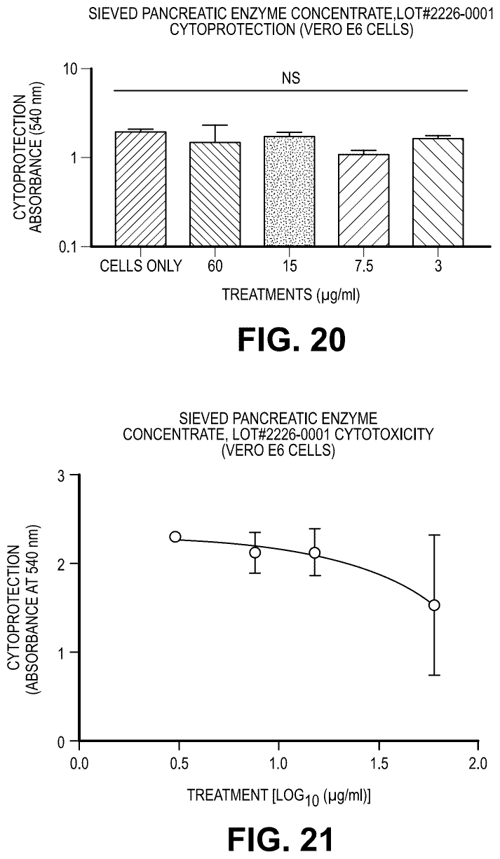

A series of baseline tests were performed to evaluate cytotoxicity of Pancreatic Enzyme Concentrate to Vero E6 cells. FIG. 20 presents the Cytoprotection of Vero E6 Cells against cell death after treatment with Pancreatic Enzyme Concentrate in lot number 2226-0001. As shown, Vero E6 cells have cytoprotection against cell death with Pancreatic Enzyme Concentrate in concentrations of 15, 7.5 and 3.0 micrograms (μg) per milliliter as is shown by the fact that treated cells show the same viability as untreated controls (no significant difference between viability of treated and untreated cells). At a concentration of 60 μg per milliliter the treatment was slightly cytotoxic to Vero E6 cells but is still at a dosing level otherwise safe for use in humans and mammals. FIG. 21, Cytotoxicity of Vero E6 Cells after treatment with Pancreatic Enzyme Concentrate (Lot number 2226-0001), presents these same results as a function of treatment concentration. The above data are acquired using the neutral red cytoprotection assay.

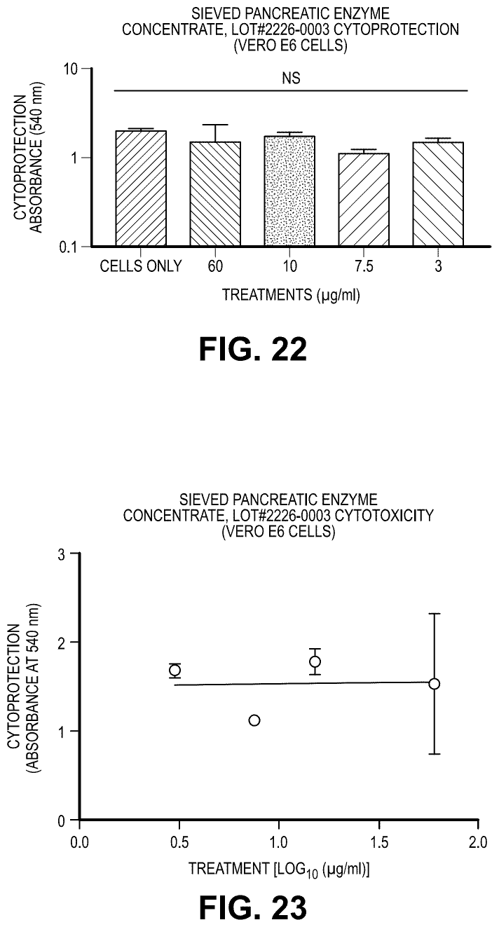

FIG. 22 presents the Cytoprotection of Vero E6 Cells against cell death after treatment with Pancreatic Enzyme Concentrate in a second lot of pancreatin (Lot 2226-0003). As shown, Vero E6 cells have cytoprotection against cell death with Pancreatic Enzyme Concentrate in concentrations of 15, 7.5 and 3.0 μg per milliliter with a statistically non-significant difference compared with the untreated cells. At a concentration of 60 μg per milliliter the treatment did have some cytotoxicity to Vero E6 cells but is still at a dosing level otherwise safe for use in humans and mammals. FIG. 23, Cytotoxicity of Vero E6 Cells after treatment with Pancreatic Enzyme Concentrate lot number 2226-0003, presents these same results as a function of treatment concentration. The above data are acquired using the neutral red cytoprotection assay.

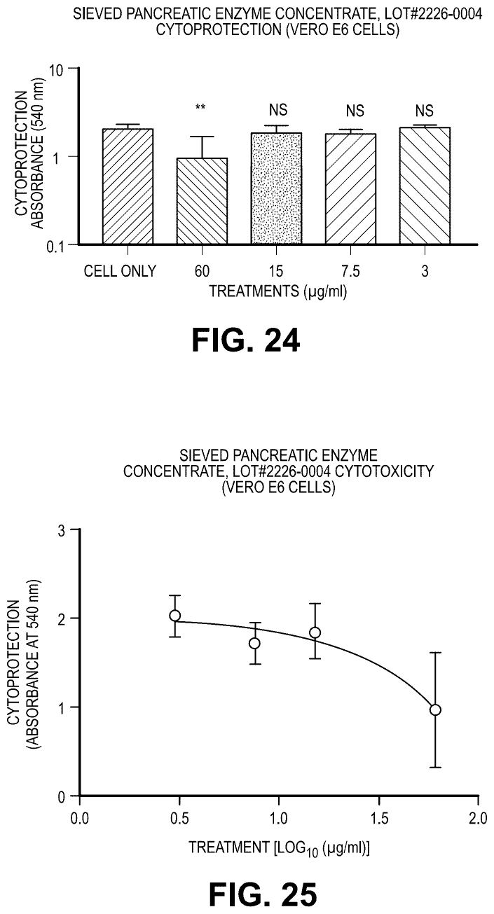

FIG. 24 presents the Cytoprotection of Vero E6 Cells against cell death after treatment with ka third lot of Pancreatic Enzyme Concentrate (Lot 2226-0004). Once again, as shown, Vero E6 cells have cytoprotection against cell death with Pancreatic Enzyme Concentrate in concentrations of 15, 7.5 and 3.0 μg per milliliter with a statistically non-significant difference compared with the untreated cells. At a concentration of 60 μg per milliliter the treatment did have some cytotoxicity to Vero E6 cells but is still at a dosing level otherwise safe for use in humans and mammals. FIG. 25, Cytotoxicity of Vero E6 Cells after treatment with Pancreatic Enzyme Concentrate lot number 2226-0004, presents these same results as a function of treatment concentration. The above data are acquired using the neutral red cytoprotection assay.

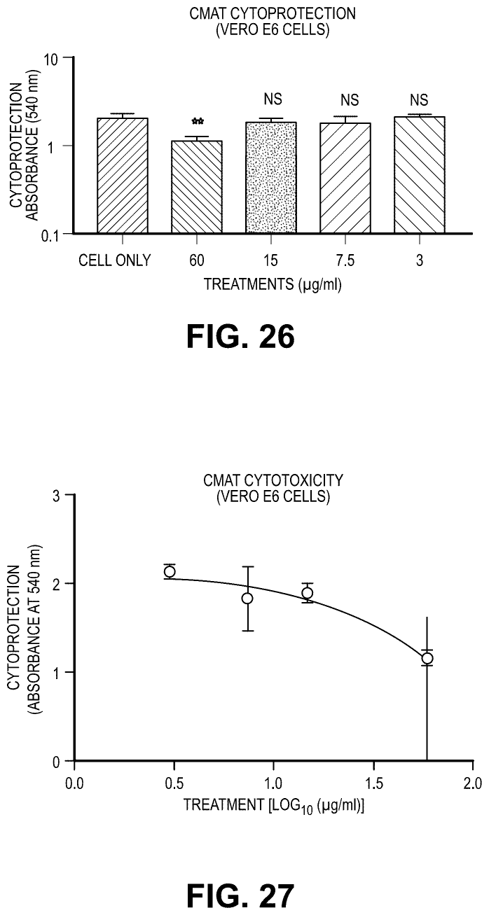

FIG. 26 presents the Cytoprotection of Vero E6 Cells against cell death after treatment with Microencapsulated Pancreatic Enzyme Concentrate. As shown, Vero E6 cells have cytoprotection against cell death with Microencapsulate Pancreatic Enzyme Concentrate in concentrations of 15, 7.5 and 3.0 μg per milliliter with a statistically non-significant difference compared with the untreated cells. Microencapsulated Pancreatic Enzyme Concentrate only showed a cytotoxicity at 60 μg/mL. Data was analyzed using an unpaired t-test compared to the cells only control. **P=0.0016-0.0080.

### Experiment 2

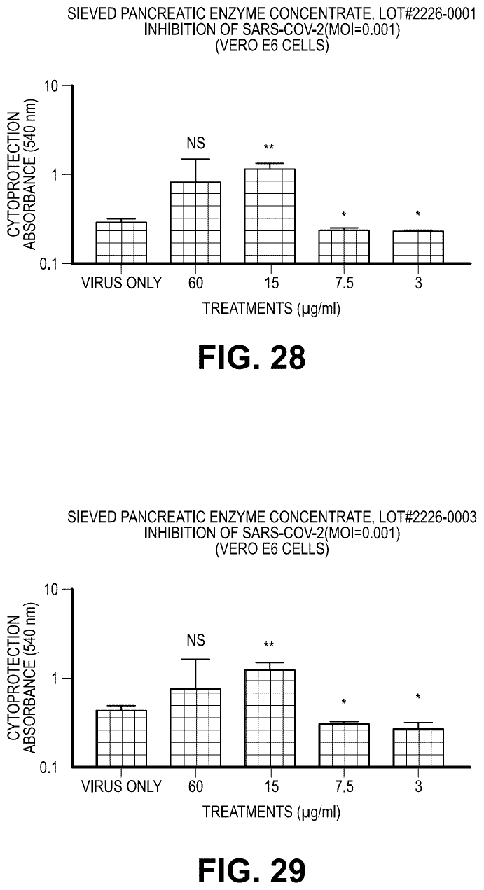

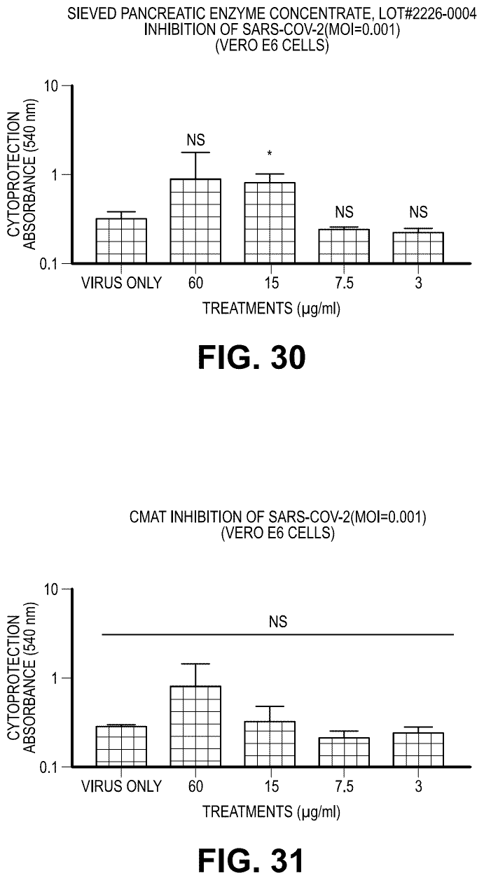

A series of tests were performed to evaluate the Inhibition of SARS-CoV-2 infectivity by Pancreatic Enzyme Concentrate and Microencapsulated Pancreatic Enzyme Concentrate using Vero E6 Cells with Multiples of Infection (MOI)=0.001 and MOI=0.0001. FIG. 28 presents the Inhibition of SARS-CoV-2 in Vero E6 Cells with Multiple of Infection (MOI)=0.001 after treatment with Pancreatic Enzyme Concentrate lot number 2226-0001. Similarly, FIG. 29 presents the Inhibition of SARS-CoV-2 in Vero E6 Cells with Multiple of Infection (MOI)=0.001 after treatment of the virus with Pancreatic Enzyme Concentrate lot number 2226-0003 and FIG. 30 presents the Inhibition of SARS-CoV-2 in Vero E6 Cells with Multiple of Infection (MOI)=0.001 after treatment of the virus with Pancreatic Enzyme Concentrate lot number 2226-0004. At a Concentration of 15 mg/ml Pancreatic Enzyme Concentrate is cytoprotective in the three tested lots above of Pancreatic Enzyme Concentrate indicating a statistically relevant reduction in virus infection (antiviral). Data were analyzed using an unpaired t test compared to the virus only control. *P=0.0287, ***P=0.002-0.004, ****P<0.0001. The above data are acquired using the neutral red cytoprotection assay.

FIG. 31 presents the Inhibition of SARS-CoV-2 in Vero E6 Cells with Multiple of Infection (MOI)=0.001 after treatment with Microencapsulated Pancreatic Enzyme Concentrate. While statistical inhibition was not shown, this is likely due to the diffusion required from the inert coating coupled with a 20% reduction by weight of active enzyme. It should be noted that significance was shown at an MOI=0.0001 (See FIG. 35).

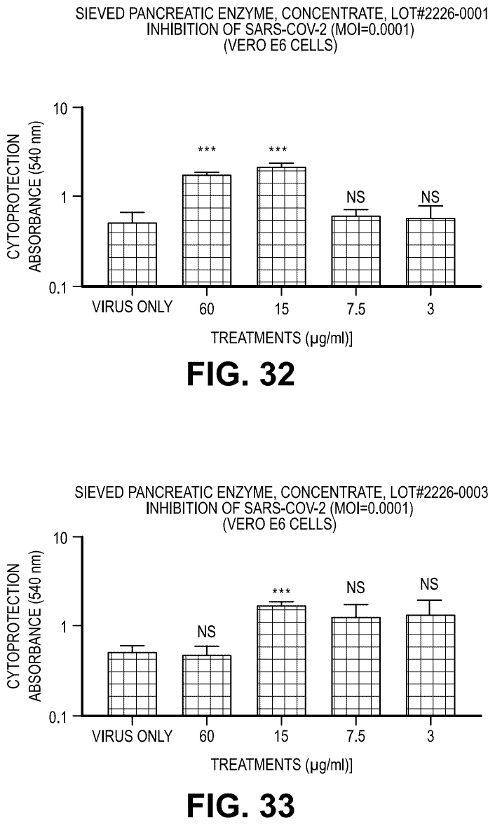

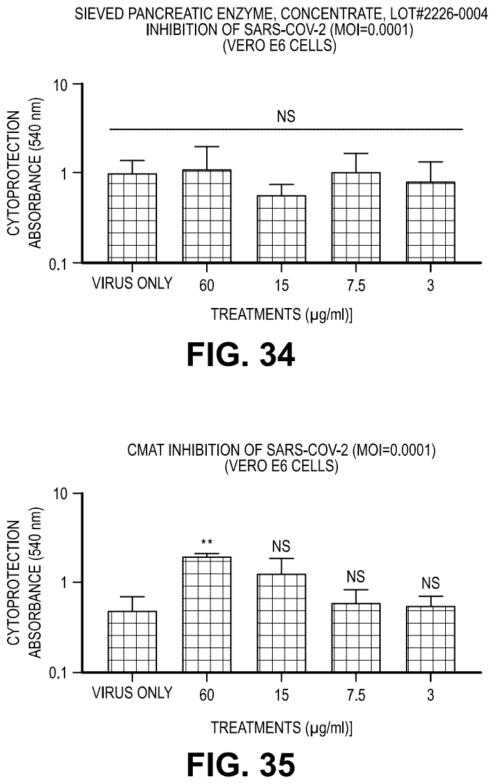

FIG. 32 presents the Inhibition of SARS-CoV-2 in Vero E6 Cells with Multiple of Infection (MOI)=0.0001 after treatment with Pancreatic Enzyme Concentrate lot number 2226-0001. Similarly, FIG. 33 presents the Inhibition of SARS-CoV-2 in Vero E6 Cells with Multiple of Infection (MOI)=0.0001 after treatment of the virus with Pancreatic Enzyme Concentrate lot number 2226-0003 and FIG. 34 presents the Inhibition of SARS-CoV-2 in Vero E6 Cells with Multiple of Infection (MOI)=0.0001 after treatment with Pancreatic Enzyme Concentrate lot number 2226-0004. At a concentration level of 15 mg/ml is cytoprotective in lots number 2226-0001 and lot 2226-0003 of Pancreatic Enzyme Concentrate indicating a statistically relevant reduction in virus infection (antiviral). Data were analyzed using an unpaired t test compared to the virus only control. *P=0.0287, ***P=0.002-0.004, **** P<0.0001.

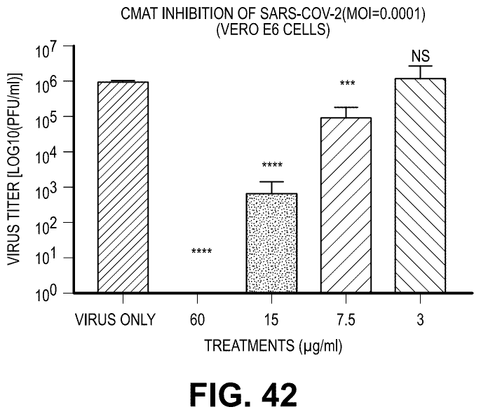

FIG. 35 presents the Inhibition of SARS-CoV-2 in Vero E6 Cells with Multiple of Infection (MOI)=0.0001 after treatment with Microencapsulated Pancreatic Enzyme Concentrate. Statistical variance in the data, likely due to self-cytotoxicity, makes interpretation of the cytoprotection data for CMAT concentrations of 3-15 μg, it is clear there is a statistically significant cytoprotective effect in the 60 μg dose. This is confirmed in FIG. 42, which presents the Virus Titer of SARS-CoV-2 in Vero E6 Cells with a Multiple of Infection (MOI)=0.0001 after treatment with Microencapsulated Pancreatic Enzyme Concentrate. As will be shown, there is a 1,000 times reduction in active virus at a dosing of 15 μg/mL.

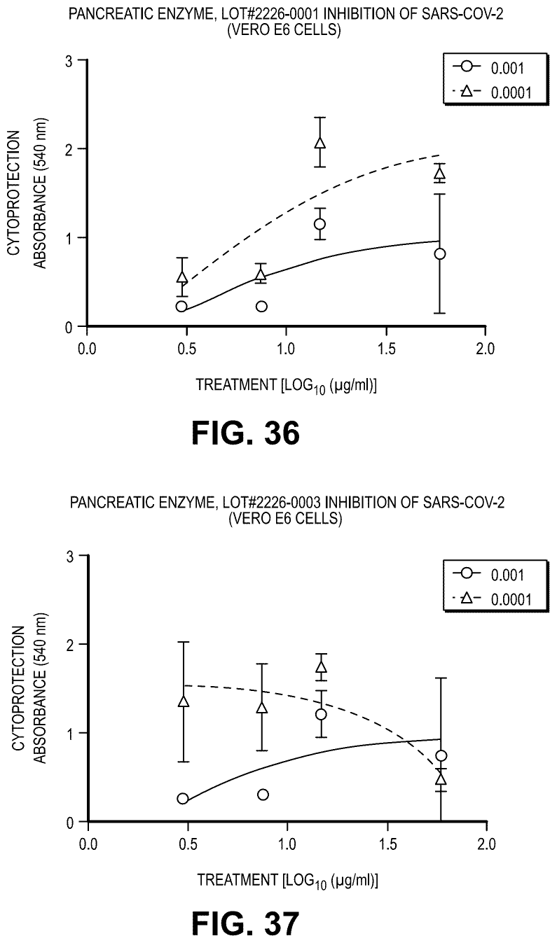

FIG. 36 presents a summary of the Inhibition of SARS-CoV-2 in Vero E6 Cells with Multiples of Infection (MOI)=0.0001 and MOI=0.001 after treatment of the virus with Pancreatic Enzyme Concentrate lot number 2226-0001. A shown, cytoprotection and SARS-CoV-2 increases with increasing treatment concentration.

FIG. 37 presents a summary of the Inhibition of SARS-CoV-2 in Vero E6 Cells with Multiples of Infection (MOI)=0.0001 and MOI=0.001 after treatment with Pancreatic Enzyme Concentrate lot number 2226-0003. A shown, cytoprotection and SARS-CoV-2 also increases with increasing treatment concentration until higher concentrations, where results are skewed by pancreatic Enzyme concentrate dosing inducing cytotoxicity to Vero E6 cells.

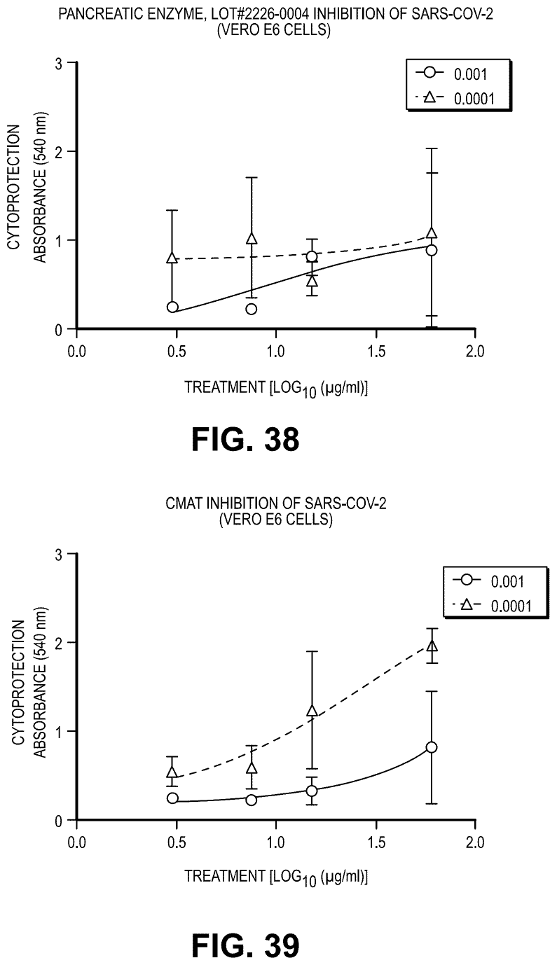

FIG. 38 presents a summary of the Inhibition of SARS-CoV-2 in Vero E6 Cells with Multiples of Infection (MOI)=0.0001 and MOI=0.001 after treatment with Pancreatic Enzyme Concentrate lot number 2226-0004. Once again, as shown, cytoprotection and SARS-CoV-2 increases with increasing treatment concentration.

FIG. 39 presents a summary of the Inhibition of SARS-CoV-2 in Vero E6 Cells with Multiples of Infection (MOI)=0.0001 and MOI=0.001 after treatment with Microencapsulated Pancreatic Enzyme Concentrate. Once again, as shown, cytoprotection and SARS-CoV-2 increases with increasing treatment concentration.

### Results Summary

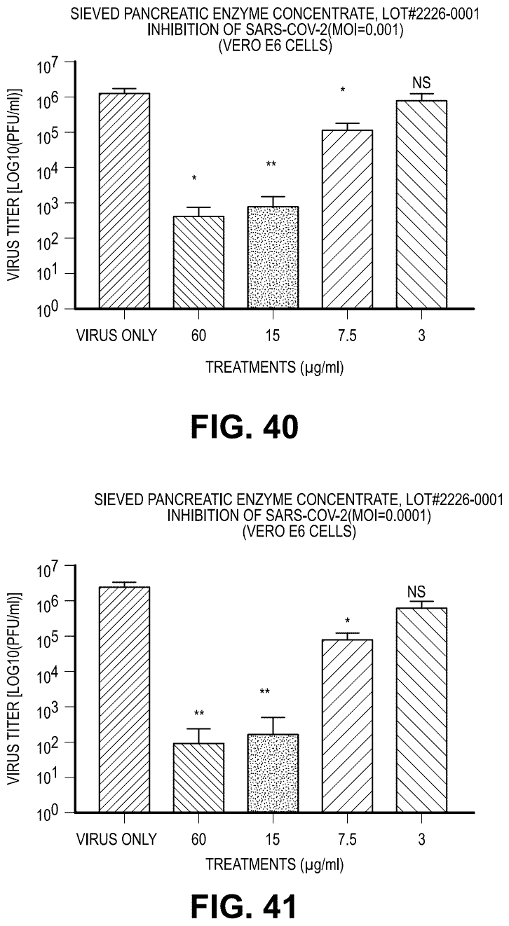

Significant virus reductions were obtained at 60, 15 and 7.5 μg/mL with Pancreatic Enzyme Concentrate. FIG. 40 presents the Virus Titer of SARS-CoV-2 in Vero E6 Cells with a Multiple of Infection (MOI)=0.001 after treatment with Pancreatic Enzyme Concentrate lot number 2226-0001. As shown, there is well over a 1000 times reduction in active virus at a dosing of 60 μg/mL as well as at a doing of 15 μg/mL.

FIG. 41 presents the Virus Titer of SARS-CoV-2 in Vero E6 Cells with a Multiple of Infection (MOI)=0.0001 after treatment with Pancreatic Enzyme Concentrate lot number 2226-0001. Once again significant virus reductions were obtained at 60, 15 and 7.5 μg/mL with Pancreatic Enzyme Concentrate. At a doing of 15 μg/mL there is over a 10,000 times reduction in active virus.

For Microencapsulated Pancreatic Enzyme Concentrate, concentrations 15 and 7.5 ug/ml showed significant virus reduction, while 60 mg/ml was inhibitory possibly due to cytotoxicity since this was observed in the cytoprotection/cytotoxicity assay as well. FIG. 42 presents the Virus Titer of SARS-CoV-2 in Vero E6 Cells with a Multiple of Infection (MOI)=0.0001 after treatment with Microencapsulated Pancreatic Enzyme Concentrate. As shown, there is a 1,000 times reduction in active virus at a dosing of 15 μg/mL.

### CONCLUSION

At a minimum, these aforecited tests demonstrate that concentrations of 7.5 and 15 μg/mL of Pancreatic Enzyme Concentrate and Microencapsulated Pancreatic Enzyme Concentrate inhibit the SARS-CoV-2 virus. This interpretation would be supported by the cytoprotection assay. **P=0.0045-0.0076.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

What is claimed is:

1. A method for treatment, prophylaxis, mitigation, or accelerated recovery of a coronavirus infection in a subject, comprising administering to the subject a pharmaceutical composition that comprises digestive enzymes.

2. The method of claim 1, wherein the digestive enzymes are coated.

3. The method of claim 1, wherein the coronavirus infection is SARS-CoV-2 or a variant thereof.

4. The method of claim 1, wherein the subject has COVID-19.

5. The method of claim 1, wherein the digestive enzymes comprise a protease, a lipase, an amylase, or a combination thereof.

6. The method of claim 1, wherein the digestive enzymes are animal enzymes from a mammal.

7. The method of claim 6, wherein the mammal is a pig.

8. The method of claim 6, wherein the digestive enzymes are animal enzymes from a pancreas of the mammal.

9. The method of claim 1, wherein the pharmaceutical composition is an oral, a nasal, a rectal, a parenteral, a percutaneous endoscopic gastrostomy (PEG), an esophagogastroduodenoscopy (EGD), or a gastrostomy (G-tube) formulation.

10. The method of claim 9, wherein the pharmaceutical composition comprises a buffer, an excipient, or a combination thereof.

11. The method of claim 1, wherein the pharmaceutical composition is a dosage formulation selected from the group consisting of a pill, a solution, a tablet, a capsule, a mini-tab, a sprinkle, and a combination thereof.

12. The method of claim 1, wherein the pharmaceutical composition is a nasal spray, a nasal drop, or a nasal wash.

13. The method of claim 1, wherein the pharmaceutical composition comprises an oral rinse or a mouth wash.

14. The method of claim 1, wherein administration comprises a percutaneous endoscopic gastrostomy (PEG), esophagogastroduodenoscopy (EGD), or gastrostomy (G-tube) insertion.

15. The method of claim 1, wherein the pharmaceutical composition is a suppository.

16. The method of claim 5, wherein a total amount of protease in the pharmaceutical composition ranges from about 10,000 to about 1,500,000 United States Pharmacopeia (U.S.P.) units/dose.

17. The method of claim 5, wherein a total amount of lipase in the pharmaceutical composition ranges from about 1,500 to about 282,000 U.S.P. units/dose.

18. The method of claim 5, wherein a total amount of amylase in the pharmaceutical composition ranges from about 1,000 to about 15,000,000 U.S.P. units/dose.

19. The method of claim 5, wherein the digestive enzymes comprise at least one protease and at least one lipase, and wherein a total protease and a total lipase in the pharmaceutical composition in U.S.P. units are present in a ratio of protease to lipase of from about 1:1 to about 20:1.

20. The method of claim 19, wherein the total protease and the total lipase in the pharmaceutical composition in U.S.P. units are present in a ratio of protease to lipase of from about 4:1 to about 10:1.

21. The method of claim 5, wherein the digestive enzymes comprise at least one protease and at least one amylase, and wherein a total protease and a total amylase in the pharmaceutical composition in U.S.P. units are present in a ratio of protease to amylase of from about 1:0.1 to about 1:10.

22. The method of claim 2, wherein digestive enzymes comprise coated digestive enzyme particles, and wherein the coated digestive enzyme particles comprise (i) a core comprising the digestive enzymes and (ii) a coating.

23. The method of claim 2, wherein the digestive enzymes are coated with a polymer or an enteric coating.

24. The method of claim 23, wherein the digestive enzymes are coated with the polymer, and the polymer comprises one or more of a cellulose acetate phthalate (CAP), a cellulose acetate trimellitate (CAT), a hydroxyl propyl methyl cellulose phthalate (HPMCP), a hydroxyl propyl methyl cellulose acetate succinate (HPMCAS), a polyvinyl acetate phthalate (PVAP), a methacrylic acid copolymer, a shellac, a Zein, an ethylcellulose (EC), or a combination thereof.

25. The method of claim 2, wherein the digestive enzymes are coated with a lipid.

26. The method of claim 25, wherein the lipid comprises a pharmaceutical grade lipid or a food grade lipid.

27. The method of claim 22, wherein the digestive enzymes are present in the coated digestive enzyme particles in an amount of from about 5% to about 99% by weight.

28. The method of claim 1, wherein the digestive enzymes are animal enzymes, microbial enzymes, plant enzymes, recombinant enzymes, synthetic enzymes, or a combination thereof.

29. The method of claim 1, wherein the coronavirus comprises a Coronavirus HCoV-229E, a Human Coronavirus HCoV-NL63, a Transmissible Gastroenteritis Virus (TGEV), a Porcine Epidemic Diarrhea Virus (PEDV), a Feline Infectious Peritonitis Virus (FIPV), a Canine Coronavirus (CCoV), a Murine Hepatitis Virus (MHV), a Bovine Coronavirus (BCoV), a Severe Acute Respiratory Syndrome Coronavirus (SARS-CoV-2), a SARS-CoV-1 (SARS1), or a Middle East Respiratory Syndrome Coronavirus (MERS-CoV).

30. The method of claim 12, wherein the digestive enzymes are present in the nasal spray, the nasal drop, or the nasal wash in solution in an amount of about 1% to about 30% by weight.

31. A method of treating a gastrointestinal symptom associated with a Severe Acute Respiratory Syndrome 2 (SARS-CoV-2) infection in a subject in need thereof, comprising administering to the subject a pharmaceutical composition that comprises digestive enzymes; wherein the digestive enzymes comprise a protease, a lipase, and an amylase, and wherein the digestive enzymes are provided as pancreatin.

32. The method of claim 31, further comprising administering to the subject a gastrointestinal modulator.

33. The method of claim 32, wherein the gastrointestinal modulator comprises a Histamine-2 receptor antagonist.

34. The method of claim 32, wherein the gastrointestinal modulator comprises a Proton Pump Inhibitor (PPI).

35. The method of claim 31, wherein the gastrointestinal symptom associated with a Severe Acute Respiratory Syndrome 2 (SARS-CoV-2) infection comprises nausea, vomiting, and diarrhea, or a combination thereof.

36. The method of claim 31, wherein the subject has COVID-19.

37. The method of claim 31, wherein the pharmaceutical composition comprises a buffer, an excipient, or a combination thereof.

38. The method of claim 31, wherein the pharmaceutical composition comprises an oral composition.

39. The method of claim 31, wherein the digestive enzymes are coated.

40. The method of claim 39, wherein digestive enzymes comprise coated digestive enzyme particles, and wherein the coated digestive enzyme particles comprise (i) a core comprising the digestive enzymes and (ii) a coating.

41. The method of claim 40, wherein the digestive enzymes are present in the coated digestive enzyme particles in an amount of from about 5% to about 99% by weight.

42. The method of claim 39, wherein the digestive enzymes are coated with a lipid.

43. The method of claim 42, wherein the lipid comprises a pharmaceutical grade lipid or a food grade lipid.

44. The method of claim 31, wherein a total amount of protease in the pharmaceutical composition ranges from about 10,000 to about 1,500,000 United States Pharmacopeia (U.S.P.) units/dose.

45. The method of claim 31, wherein a total amount of lipase in the pharmaceutical composition ranges from about 1,500 to about 282,000 U.S.P. units/dose.

46. The method of claim 31, wherein a total amount of amylase in the pharmaceutical composition ranges from about 1,000 to about 15,000,000 U.S.P. units/dose.

47. The method of claim 31, wherein the digestive enzymes comprise at least one protease and at least one lipase, and wherein the total protease and the total lipase in the pharmaceutical composition in U.S.P. units are present in a ratio of protease to lipase of from about 1:1 to about 20:1.

48. The method of claim 47, wherein the total protease and the total lipase in the pharmaceutical composition in U.S.P. units are present in a ratio of protease to lipase of from about 4:1 to about 10:1.

49. The method of claim 31, wherein the digestive enzymes comprise at least one protease and at least one amylase, and wherein the total protease and the total amylase in the pharmaceutical composition in U.S.P. units are present in a ratio of protease to amylase of from about 1:0.1 to about 1:10.

50. The method of claim 31, wherein the pharmaceutical composition is a dosage formulation selected from the group consisting of a pill, a solution, a tablet, a capsule, a mini-tab, a sprinkle, and a combination thereof.

51. A method for reducing an infection rate of a Severe Acute Respiratory Syndrome (SARS-CoV-2) infection in a subject in need thereof, comprising administering to the subject a pharmaceutical composition that comprises digestive enzymes, wherein the digestive enzymes comprise a protease, an amylase, and a lipase, and wherein the digestive enzymes are provided as pancreatin, and wherein the infection rate is reduced.

52. The method of claim 51, wherein the digestive enzymes are coated.

53. The method of claim 52, wherein the digestive enzymes are coated with a lipid.

54. The method of claim 52, wherein digestive enzymes comprise coated digestive enzyme particles, and wherein the coated digestive enzyme particles comprise (i) a core comprising the digestive enzymes and (ii) a coating.

55. A method for reducing a transmission rate of a Severe Acute Respiratory Syndrome (SARS-CoV-2) infection in a subject in need thereof, comprising administering to the subject a pharmaceutical composition that comprises digestive enzymes, wherein the digestive enzymes comprise a protease, an amylase, and a lipase, and wherein the digestive enzymes are provided as pancreatin, and wherein the transmission rate is reduced.

56. The method of claim 55, wherein the digestive enzymes are coated.

57. The method of claim 56, wherein the digestive enzymes are coated with a lipid.

58. The method of claim 56, wherein digestive enzymes comprise coated digestive enzyme particles, and wherein the coated digestive enzyme particles comprise (i) a core comprising the digestive enzymes and (ii) a coating.

59. A method of treating long COVID in a subject in need thereof, comprising administering to the subject a pharmaceutical composition that comprises digestive enzymes, wherein the digestive enzymes comprise a protease, an amylase, and a lipase, and wherein the digestive enzymes are provided as pancreatin, and wherein long COVID is treated.

60. The method of claim 59, wherein the digestive enzymes are coated.

61. The method of claim 60, wherein the digestive enzymes are coated with a lipid.

62. The method of claim 60, wherein digestive enzymes comprise coated digestive enzyme particles, and wherein the coated digestive enzyme particles comprise (i) a core comprising the digestive enzymes and (ii) a coating.

63. The method of claim 59, wherein a symptom of long COVID is reduced following administration of the pharmaceutical composition.

64. The method of claim 63, wherein the symptom is fatigue, shortness of breath or difficulty breathing, cough, joint pain, chest pain, memory, concentration or sleep problems, muscle pain or headache, fast or pounding heartbeat, loss of smell or taste, depression or anxiety, fever, dizziness when you stand, or worsened symptoms after physical or mental activities.

65. A method of enhancing cytoprotection against a Severe Acute Respiratory Syndrome (SARS-CoV-2) infection in a subject in need thereof, comprising administering to the subject a pharmaceutical composition that comprises digestive enzymes, wherein the digestive enzymes comprise a protease, an amylase, and a lipase, and wherein the digestive enzymes are provided as pancreatin, and wherein cytoprotection is enhanced.

66. The method of claim 65, wherein the digestive enzymes are coated.

67. The method of claim 66, wherein the digestive enzymes are coated with a lipid.

68. The method of claim 66, wherein digestive enzymes comprise coated digestive enzyme particles, and wherein the coated digestive enzyme particles comprise (i) a core comprising the digestive enzymes and (ii) a coating.

69. A method of reducing a virus titer of a Severe Acute Respiratory Syndrome (SARS-CoV-2) in a subject in need thereof, comprising administering to the subject a pharmaceutical composition that comprises digestive enzymes, wherein the digestive enzymes comprise a protease, an amylase, and a lipase, and wherein the digestive enzymes are provided as pancreatin, and wherein the virus titer is reduced.

70. The method of claim 69, wherein the digestive enzymes are coated.

71. The method of claim 70, wherein the digestive enzymes are coated with a lipid.

72. The method of claim 70, wherein digestive enzymes comprise coated digestive enzyme particles, and wherein the coated digestive enzyme particles comprise (i) a core comprising the digestive enzymes and (ii) a coating.
